SEQUENCE LISTING (3 sequences)

SEQ ID NO: 1 - protein, 608 aa, Artificial Sequence
  55kDa Recombinant Collagen Fragment
MYRNLIIATALTCGAYSAYVPSEPWSTLTPDASLESALKDYSQTFGIAIKSLDADKIKRDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 2 - protein, 546 aa, Artificial Sequence
  50kDa Recombinant Collagen Fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 3 - protein, 62 aa, Artificial Sequence
  Signal Sequence
MYRNLIIATALTCGAYSAYVPSEPWSTLTPDASLESALKDYSQTFGIAIKSLDADKIKRDSY

(19) United States
(12) Patent Application Publication
(10) Pub. No.: US 2024/0099955 A1
(43) Pub. Date: Mar. 28, 2024

(54) SKINCARE COMPOSITIONS AND METHODS OF USE THEREOF

(71) Applicant: MODERN MEADOW, INC., Nutley, NJ (US)

(72) Inventors: Yelena ZOLOTARSKY, Nutley, NJ (US); Lixin DAI, Nutley, NJ (US); Dan DUEPPEN, Nutley, NJ (US)

(21) Appl. No.: 18/263,804

(22) PCT Filed: Feb. 4, 2022
(86) PCT No.: PCT/US2022/015342

§ 371 Date: Aug. 1, 2023

Related U.S. Application Data
(60) Provisional application No. 63/146,076, filed on Feb. 5, 2021.

(51) Int. Cl. A61K 8/65; A61K 38/39; A61Q 19/00; C07K 14/78; C12P 21/02

(52) U.S. Cl. CPC A61K 8/65; A61K 38/39; A61Q 19/00; C07K 14/78; C12P 21/02

(57) ABSTRACT

The present disclosure relates to a recombinant collagen fragment having a molecular weight of about 55 kDa, and compositions comprising the recombinant collagen fragment. Methods of producing the recombinant collagen fragment and methods of treatment using the recombinant collagen fragment are also provided.

The content of the electronically submitted sequence listing (Name: 4431_061PC01_Seqlisting_ST25.txt; Size: 10,959 Bytes; and Date of Creation: Feb. 4, 2022) is herein incorporated by reference in its entirety.

### BACKGROUND

It is well appreciated that aging and environmental stress cause changes in, among other things, the appearance, elasticity, and thickness of skin. In particular, ageing and environmental factors can lead to dermatological conditions including, but not limited to fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, and rhinophyma. Consequently, consumers are increasingly turning to skincare products to reduce the appearance of wrinkles, even out skin tone, provide moisture, reduce the appearance of dark circles under the eyes, and more.

Although there are numerous skincare products on the market to improve skin appearance, many consumers are hesitant to use chemically-synthesized products they perceive as being environmentally unfriendly or otherwise unsafe. Consequently, there is a need for skincare products containing natural products which are perceived as more acceptable than chemically-synthesized products.

### BRIEF SUMMARY

In some embodiments, this disclosure provides a recombinant collagen fragment having a molecular weight of about 55 kDa and a sequence identity of at least about 85% to the amino acid sequence set forth in SEQ ID NO: 1. In some embodiments, the collagen fragment has the amino acid sequence set forth in SEQ ID NO: 1.

In some embodiments, this disclosure provides a composition comprising a recombinant collagen fragment having a molecular weight of about 55 kDa and a sequence identity of at least about 85% to the amino acid sequence set forth in SEQ ID NO: 1. In some embodiments, the composition comprises the collagen fragment having the amino acid sequence set forth in SEQ ID NO: 1. In some embodiments, the composition further comprises a recombinant collagen fragment having the amino acid sequence set forth in SEQ ID NO: 2. In some embodiments, the composition further comprises a pharmaceutically acceptable or cosmetically acceptable excipient.

In some embodiments, this disclosure provides a method of treating a dermatological condition comprising topically administering an effective amount of a recombinant collagen fragment having a molecular weight of about 55 kDa and a sequence identity of at least about 85% to the amino acid sequence set forth in SEQ ID NO: 1 to a subject in need thereof. In some embodiments, this disclosure provides a method of treating a dermatological condition comprising topically administering an effective amount the collagen fragment having the amino acid sequence set forth in SEQ ID NO: 1 to a subject in need thereof.

In some embodiments, this disclosure provides a method of treating a dermatological condition comprising topically administering an effective amount of the composition comprising a recombinant collagen fragment having a molecular weight of about 55 kDa and a sequence identity of at least about 85% to the amino acid sequence set forth in SEQ ID NO: 1 to a subject in need thereof. In some embodiments, this disclosure provides a method of treating a dermatological condition comprising topically administering an effective amount of the composition comprising the collagen fragment having the amino acid sequence set forth in SEQ ID NO: 1 to a subject in need thereof.

In some embodiments, the dermatological condition is selected from the group consisting of fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, rhinophyma, and combinations thereof.

In some embodiments, the composition is topically administered to an area of skin selected from the group consisting of a facial surface, scalp, neck, ears, shoulders, chest (including breasts and/or the décolletage), arms, hands, legs, stomach, buttocks, groin, back, feet, and combinations thereof.

In some embodiments, this disclosure also provides methods of producing the recombinant collagen fragments disclosed herein, comprising producing the recombinant collagen fragment in a genetically engineered strain of yeast. In some embodiments, the yeast is Pichia pastoris. In some embodiments, the method comprises: fermenting a genetically engineered yeast in a fermentation broth; recovering from the supernatant of the fermentation broth recombinant collagen fragments secreted by the genetically engineered yeast; and optionally, purifying the recombinant collagen fragments. In some embodiments, the method comprises: fermenting a genetically engineered yeast in a fermentation broth; recovering recombinant collagen fragments not secreted into the supernatant by the genetically engineered yeast; and optionally, purifying the recombinant collagen fragments. In some embodiments, recovering the recombinant collagen fragments not secreted into the supernatant comprises recovering the intracellular recombinant collagen fragments from a cell paste. In some embodiments, the methods described herein further comprise lyophilizing the recovered recombinant collagen fragments.

In some embodiments, this disclosure also provides a skincare product comprising a recombinant collagen fragment having a molecular weight of about 55 kDa and a sequence identity of at least about 85% to the amino acid sequence set forth in SEQ ID NO: 1 for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof. In some embodiments, this disclosure also provides a skincare product comprising a recombinant collagen fragment having the amino acid sequence set forth in SEQ ID NO: 1 for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

In some embodiments, this disclosure also provides a skincare product comprising compositions comprising a recombinant collagen fragment having a molecular weight of about 55 kDa and a sequence identity of at least about 85% to the amino acid sequence set forth in SEQ ID NO: 1, and a skincare product comprising compositions comprising a recombinant collagen fragment having the amino acid sequence set forth in SEQ ID NO: 1, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

In some embodiments, this disclosure also provides a method of treating a wound in a human subject in need thereof, the methods comprising applying the composition comprising a recombinant collagen fragment having a molecular weight of about 55 kDa and a sequence identity of at least about 85% to the amino acid sequence set forth in SEQ ID NO: 1 to the wound on the subject, wherein applying the recombinant collagen fragment induces the production of human Type I collagen, human Type III collagen, or a combination thereof. In some embodiments, this disclosure also provides a method of treating a wound in a human subject in need thereof, comprising applying the composition comprising a recombinant collagen fragment having the amino acid sequence set forth in SEQ ID NO: 1 to the wound on the subject, wherein applying the recombinant collagen fragment induces the production of human Type I collagen, human Type III collagen, or a combination thereof. In some embodiments, the collagen fragment is topically applied to the wound.

In some embodiments, this disclosure also provides an aqueous skincare composition comprising water and the recombinant collagen fragment having a molecular weight of about 55 kDa and a sequence identity of at least about 85% to the amino acid sequence set forth in SEQ ID NO: 1, at least one stress related protein, at least one cell wall related protein, at least one DNA or protein synthesis related protein, at least one metabolic enzyme, and, optionally, at least one cell integrity protein. In some embodiments, this disclosure also provides an aqueous skincare composition comprising water and the recombinant collagen fragment having the amino acid sequence set forth in SEQ ID NO: 1, at least one stress related protein, at least one cell wall related protein, at least one DNA or protein synthesis related protein, at least one metabolic enzyme, and, optionally, at least one cell integrity protein. In some embodiments, this disclosure also provides methods of treating an area of skin comprising topically applying an effective amount of the aqueous skincare compositions described herein, to the area of skin.

In some embodiments, the area of skin is selected from the group consisting of a facial surface, scalp, neck, ears, shoulders, chest (including breasts and/or the décolletage), arms, hands, legs, stomach, buttocks, groin, back, feet, and combinations thereof. In some embodiments, the facial surface is selected from the group consisting of forehead, eyes, a perioral surface, a chin surface, a periorbital surface, a nasal surface, a cheek skin surface, and combinations thereof.

### BRIEF DESCRIPTION OF THE FIGURES

FIG. 1 is a flow chart showing the experimental approach to recovering intracellular recombinant collagen fragments for experiments 023 and 027, as described in Example 3.

FIGS. 2A-C show expression of the extracellular recombinant collagen fragments from the experiments described in Example 3. FIG. 2A shows a 50 kDa collagen fragment, and FIGS. 2B and 2C show 50 kDa and 55 kDa collagen fragments.

FIGS. 3A and 3B show the percent of intracellular collagen that was recovered during experiments 023 and 027, as described in Example 3.

FIGS. 4A-4C show the overall total recovery yields for secreted collagen (supernatant only) as compared to the yields from experiments 023 (fermentation broth) and 027 (cell paste).

### DETAILED DESCRIPTION

### Definitions

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used herein, the term “about” used with numerical values means “within 10% of the stated value,” unless expressly noted otherwise. For example, “about 5% by weight” means from 4.5% by weight to 5.5% by weight.

As used herein, the term “recombinant collagen” refers to refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX, prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties). In some embodiments, the collagen described herein can be prepared using bovine Type I collagen. Collagens are characterized by a repeating triplet of amino acids, -(Gly-X-Y)n-, so that approximately one-third of the amino acid residues in collagen are glycine. X is often proline and Y is often hydroxyproline. The structure of collagen may consist of three intertwined peptide chains of differing lengths.

As used herein, the phrase “stress related protein” refers to proteins which exhibit increased expression after exposure to environmental stress conditions. Environmental stress conditions include, but are not limited to, infection, inflammation, exposure to toxins (e.g., ethanol, arsenic, trace metals, ultraviolet light, etc.), starvation, hypoxia, or water deprivation. Examples of stress related proteins include, but are not limited to, heat shock proteins (Hsp), thioredoxin, ATPases, and protein disulfide isomerases.

As used herein, the phrase “cell wall related protein” refers to proteins which are found in cell walls and are involved in creation of the cell wall. Examples of cell wall proteins include, but are not limited to, exo-1,3-beta-glucanase, endo-beta-1,3-glucanase, glycosidase, 1,3-beta-glucanosyltransferase, chitin deacetylase, and fructose 1,6-bisphosphate aldolase.

As used herein, the phrase “DNA or protein synthesis related protein” refers to proteins involved in the synthesis of new deoxyribonucleic acids (“DNA”), DNA replication, transcription, translation, and protein folding. Examples of DNA or protein synthesis related proteins include, but are not limited to, elongation factor 1-alpha, adenosine kinase, 60S acidic ribosomal protein P2-A, nucleoside diphosphate kinase, ribosomal protein 51, ribosomal protein L30, ribosomal protein 59, eukaryotic translation initiation factor 5A, translation initiation factor elF4A, DNA topoisomerase 2, histone methyl transferase, S-adenosyl-L-methionine-dependent tRNA, eukaryotic translation initiation factor 3 subunit 1, lysine-tRNA ligase, DNA-directed RNA polymerase subunit beta, transcription factor II, and DNA ligase.

As used herein, the phrase “metabolic enzyme” refers to an enzyme that regulates cellular metabolism. Examples of metabolic enzymes include, but are not limited to, enolase I, triosephosphate isomerase, superoxide dismutase, glyceraldehyde-3-phosphate dehydrogenase, cobalamin-independent methionine synthase, 6-phosphogluconate dehydrogenase, phosphotransferase, NADPH-dependent alpha-keto amide reductase, phosphoglycerate mutase, cytoplasmic inorganic pyrophosphatase, alcohol dehydrogenase, carboxypeptidase Y inhibitor, phosphatidylglycerol/phosphatidylinositol transfer protein, cytoplasmic ATPase, mitochondrial matrix ATPase, peptidyl-prolyl cis-trans isomerase, adenosine kinase, thiol-specific peroxiredoxin, ornithine aminotransferase, ketol-acid reductoisomerse, aconitate hydratase, malate dehyrdrogenase, glycerol kinase, alanine: glyoxylate aminotransferase, nucleoside diphosphate kinase, chitin deacetylase, transketolase, NADPH oxidoreductase, fructose 1,6-bisphosphate aldolase, NADH-cytochrome b5 reductase, phosphomannomutase, mitochondrial peroxiredoxin, 3-isopropylmalate dehydrogenase, thioredoxin peroxidase, squalene epoxidase, malate dehydrogenase, coproporphyrinogen III oxidase, isocitrate dehydrogenase [NAD] subunit, catalytic subunit of NatB N-terminal acetyltransferase, carboxypeptidase, triacylglycerol lipase, adenosylhomocysteinase, flavin-containing monooxygenase, acid trehalase, high affinity methionine permease, subunit of alpha-1,6 mannosyltransferase complex, adenylate cyclase, phosphotransferase, methylenetetrahydrofolate reductase, alternative oxidase, Cu(+2)-transporting P-type ATPase, GTPase, 2-(3-amino-3carboxypropyl) histidine synthase subunit 1, peroxisome biosynthesis protein PAS1, folylpolyglutamate synthase, pyruvate kinase, serine/threonine-protein phosphatase, protein serine/threonine kinase, beta-mannosyltransferase 2, urea amidolyase, citrate synthase, and lanosterol synthase.

As used herein, the term “cell integrity protein” refers to proteins that help maintain the regular structure and function of a cell. Examples of cell integrity proteins include, but are not limited to, mitogen-activated protein kinases (MAPKs) such as Ste11p, Ssk2p, Ssk22p, Bcklp, Ste7p, Pbs2p, Mkklp, Mkk2p, Fus3p, Ksslp, Hoglp, Slt2p, and Smk1p.

a. Recombinant Collagen

In some embodiments, the recombinant collagen described herein is a recombinant collagen fragment. A recombinant collagen fragment can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n).

Exemplary collagen sequences from which fragments can be derived include amino acid sequences of Col1A1, Col1A2, and Col3A1, such as those described by Accession Nos. P02461.4 (SEQ ID NO: 982; human Col3A1) (www.ncbi.nlm.nih.gov/protein/124056490), NP 001029211.1 (SEQ ID NO: 978; bovine Col1A1) (www.ncbi.nlm.nih.gov/protein/77404252), NP 776945.1 (SEQ ID NO: 979; bovine Col1A2) (www.ncbi.nlm.nih.gov/protein/27806257) and NP 001070299.1 (SEQ ID NO: 980; bovine Col3A1) (www.ncbi.nlm.nih.gov/protein/116003881), which are incorporated herein by reference.

In some embodiments, the collagen fragment disclosed herein can have a molecular weight from about 40 kDa to about 60 kDa. In some embodiments, the collagen fragment can have a molecular weight of about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, or about 60 kDa. In a particular embodiment, the collagen fragment can have a molecular weight of about 55 kDa.

In some embodiments, the collagen fragment described herein can have an amino acid sequence according to SEQ ID NO: 1. In some embodiments, the collagen fragment can have at least about 70%, at least about 75%, at least about 80%, about 85%, at least about 87.5%, at least about 90%, at least about 92.5%, at least about 95%, at least about 97.5%, at least about 98%, at least about 99% or 100% sequence identity, or similarity to SEQ ID NO: 1.

The amino acid sequence of SEQ ID NO: 1 is:

MYRNLIIATALTCGAYSAYVPSEPWSTLTPDASLESALKDYSQTFGIAIK
SLDADKIKRDSYDVKSGVAVGGLAGYPGPAGPPGPPGPPGTSGHPGSPGS
PGYQGPPGEPGQAGPSGPPGPPGAIGPSGPAGKDGESGRPGRPGERGLPG
PPGIKGPAGIPGFPGMKGHRGFDGRNGEKGETGAPGLKGENGLPGENGAP
GPMGPRGAPGERGRPGLPGAAGARGNDGARGSDGQPGPPGPPGTAGFPGS
PGAKGEVGPAGSPGSNGAPGQRGEPGPQGHAGAQGPPGPPGINGSPGGKG
EMGPAGIPGAPGLMGARGPPGPAGANGAPGLRGGAGEPGKNGAKGEPGPR
GERGEAGIPGVPGAKGEDGKDGSPGEPGANGLPGAAGERGAPGFRGPAGP
NGIPGEKGPAGERGAPGPAGPRGAAGEPGRDGVPGGPGMRGMPGSPGGPG
SDGKPGPPGSQGESGRPGPPGPSGPRGQPGVMGFPGPKGNDGAPGKNGER
GGPGGPGPQGPPGKNGETGPQGPPGPTGPGGDKGDTGPPGPQGLQGLPGT
GGPPGENGKPGEPGPKGDAGAPGAPGGKGDAGAPGERGPPAIAGIGGEKA
GGFAPYYG

In some embodiments, the collagen fragment described herein can have an amino acid chain length from about 350 amino acids to about 600 amino acids and can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1. In some embodiments, the such a collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

b. Compositions Comprising Collagen Fragments

In some embodiments, the present disclosure provides a composition comprising one or more recombinant collagen fragments disclosed herein, and at least one pharmaceutically acceptable or cosmetically acceptable excipient. In particular embodiments, the composition can comprise a recombinant collagen fragment according to SEQ ID NO: 1, and at least one pharmaceutically acceptable or cosmetically acceptable excipient. In further embodiments, the composition can comprise a recombinant collagen fragment having at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1, and at least one pharmaceutically acceptable or cosmetically acceptable excipient. In still further embodiments, the composition can comprise a recombinant collagen fragment having about 85%, about 86%, about 87%, about 88%, about 89%, about 90%, about 91%, about 92%, about 93%, about 94%, about 95%, about 96%, about 97%, about 98%, about 99%, or 100% sequence identity, or similarity, to SEQ ID NO: 1, and at least one excipient that is suitable for use in a dietary supplement, e.g., a nutritional supplement.

In some embodiments, the composition can be prepared with about 0.1% to about 20% by volume of an about 0.5% to about 25% by weight recombinant collagen fragment solution. In some of these embodiments, the composition can comprise about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by volume of a recombinant collagen fragment solution. In some of these embodiments, the recombinant collagen fragment solution comprises about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of the recombinant collagen fragment. In some of these embodiments, the recombinant collagen fragment solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment with a sequence according to SEQ ID NO: 1

In some embodiments, the composition can comprise from about 0.0005% to about 25% by weight of a recombinant collagen fragment, from about 0.001% to about 25% by weight of a recombinant collagen fragment, from about 0.01% to about 25% by weight of a recombinant collagen fragment, from about 0.1% to about 25% by weight of a recombinant collagen fragment, from about 0.5% to about 20% by weight of a recombinant collagen fragment, from about 0.7% to about 17% by weight of recombinant collagen fragment, from about 1% to about 15% by weight of recombinant collagen fragment, from about 2% to about 12% by weight of recombinant collagen fragment, from about 2% to about 10% by weight of recombinant collagen fragment, from about 3% to about 9% by weight of recombinant collagen fragment, from about 4% to about 8% by weight of recombinant collagen fragment, or from about 5% to about 7% by weight of recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment with a sequence according to SEQ ID NO: 1.

c. Skincare

In some embodiments, the compositions described herein can be used to improve the aesthetic appearance of the skin and/or its appendages, for example, the surface appearance and/or texture of the skin. In some embodiments, the compositions described herein can be formulated for use on the body and face, hands, and feet, including as treatment for the eye area, for nails, and hair. The term “surface appearance” means the visual and/or tactile irregularities in the skin and/or in the scalp, including wrinkles and fine lines, expression lines on the forehead and in the space between the eyebrows, wrinkles and/or fine lines around the mouth, and/or slackening in the area around the lips and the top lip area (area located between the top lip and the nose), heterogeneity of the skin tone (liver spots, actinic lentigos), appearance and/or visibility of the pores, papery appearance of the skin, defects in the skin microrelief such as chicken pox or acne scars, imperfections of greasy skin (shiny appearance, etc.). The term “skin texture” can mean slack, flabby, less firm, less elastic skin, and/or skin that has sagged.

In some embodiments, the compositions described herein can be used to improve the aesthetic appearance of the skin, including improvement in the appearance of expression lines. Expression lines are produced by the effect of the stress exerted on the skin by the underlying muscles. Age and environmental factors such as exposure to sunlight, can deepen expression lines and make them permanent. Expression lines are characterized by the presence of grooves in the area around the orifices formed by the nose (nasal grooves), the mouth (perioral lines and so-called bitterness lines) and the eyes (crow's feet wrinkles), around which the skin muscles are located, and also between the eyebrows (glabella or lion wrinkles) and on the forehead.

In some embodiments, the compositions described herein can be used to improve the aesthetic appearance of the skin and/or visibility of the pores. Visibility of the pores can be due to an excess of sebum, aging, loss of firmness, slackening, stress, fatigue, unsuitable hygiene, climatic factors, or any combination thereof. The compositions described herein can tighten the pores, making them less visible.

In some embodiments, the compositions described herein can be used to improve a papery appearance of the skin and the behavior of the skin to the touch. Specifically, older skin can visually take on the appearance of cigarette paper, giving it an appearance similar to that of a sheet of papyrus. The papery appearance of the skin can be seen on the face and on the back of the hands of the elderly.

In some embodiments, the compositions described herein can be a composition for protecting, treating or caring for the face, for the hands, for the feet, or for the body, for example, day creams, night creams, makeup remover creams, anti-sun compositions, body milks for skin protection or care, after-sun milks, skincare lotions, gels, foams, artificial tanning compositions, and aftershave compositions. In some embodiments, the compositions described herein can be formulated, for example, as solutions, suspensions, lotions, creams, serums, gels, balms, gels, oils, oil in creams, micellar waters, face mists, face essences, blemish balm or complexion corrector formulas, toners (water and/or alcohol based), paints, polishes, sticks, pencils, sprays, aerosols, ointments, cleansing liquid washes, solid bars, shampoos, hair conditioners, hair styling products, pastes, foams, powders, mousses, balms, shaving creams, wipes, strips, patches, wound dressings, adhesive bandages, hydrogels, film-forming products, facial and skin masks, cosmetics (e.g. foundations, eye liners, eye shadows), exfoliators, deodorants and anti-perspirants, and the like. Exemplary formulations are provided herein.

In some embodiments, the composition described herein can be a cosmetic composition and the at least one excipient can be a cosmetically acceptable excipient. Cosmetically acceptable excipients are excipients suitable for use in a cosmetic product. Exemplary cosmetically acceptable excipients are described below.

In some embodiments, the cosmetic composition can comprise ingredients commonly used in cosmetic, e.g., skin care, eye care, nail care, and hair care products. These ingredients can include, but are not limited to, soaps, antimicrobials, anti-inflammatories, moisturizers, waxy alcohols, hydration agents, moisturizers, penetration enhancers, emulsifiers, natural or synthetic oils, solvents, fats, surfactants, detergents, gelling agents, emollients, antioxidants, fragrances, paints, polishes, fillers, thickeners, waxes, odor absorbers, dyestuff, coloring agents, powders, viscosity-controlling agents, anesthetics, anti-itch agents, botanical extracts, conditioning agents, darkening or whitening agents, humectants, mica, minerals, polyphenols, silicones or silicone derivatives such as dimethicone, sun blocks, vitamins, phytomedicinals, alcohols, such as denatured alcohols and ethanol, polyols, polyolethers, and other ingredients listed in the International Cosmetic Ingredient Dictionary and Handbook, 13th Ed. (2009), the entirety of which is incorporated herein by reference. In certain embodiments, a given ingredient can perform more than one function and can belong to more than one class.

In some embodiments, the composition described herein can be a therapeutic composition and the at least one excipient can be a therapeutically acceptable excipient. Therapeutic compositions can be useful for treating one or more conditions such as reducing or preventing the formation of scar tissue, promoting healing, promoting tissue regeneration, minimizing local inflammation, minimizing tissue rejection, and/or enhancing skin and/or hair graft integration. Therapeutically acceptable excipients are excipients that can serve as a vehicle or medium for an active substance and include excipients commonly used in therapeutic compositions, i.e., compositions useful for treating one or more conditions. Exemplary therapeutically acceptable excipients are described below.

The compositions described herein can also include one or more of the following additional components. Exemplary contemplated additional ingredients are set forth below; however, this disclosure is not limited to these exemplary additional ingredients.

In some embodiments, the composition described herein can further comprise one or more anti-wrinkle agents. An anti-wrinkle agent is a compound which produces an increase in the synthesis and/or in the activity of certain enzymes of the skin, when the composition is brought into contact with an area of wrinkled skin, e.g., on the body or face, including the eye area, which reduces the outward appearance of the wrinkles and/or fine lines. Exemplary anti-wrinkle agents include, but are not limited to, desquamating agents, antiglycation agents, nitric oxide synthase inhibitors, muscle relaxants and/or dermo-decontracting agents, agents for combating free radicals, and mixtures thereof.

Additional exemplary anti-wrinkle agents that can be included in the composition described herein include, but are not limited to, adenosine and its derivatives, retinol and its derivatives (e.g., retinyl palmitate), ascorbic acid and its derivatives (e.g., magnesium ascorbyl phosphate and ascorbyl glucoside), tocopherol and its derivatives (e.g., tocopheryl acetate), nicotinic acid and its precursors (e.g., nicotinamide), ubiquinone, glutathione and its precursors (e.g., L-2-oxothiazolidine-4-carboxylic acid), C-glycoside compounds (also known as C-glycosyl compounds) and their derivatives (e.g., a β-C-xylosyl derivative with the trade name PRO-XYLANE, plant extracts (e.g., rock samphire extracts and olive leaf extracts), plant proteins and their hydrolysates (e.g., rice or soybean protein hydrolysates), algal extracts (e.g., laminarian extracts), bacterial extracts, sapogenins (e.g., diosgenin), Dioscorea extracts (e.g., wild yam extracts), α-hydroxy acids, β-hydroxy acids (e.g., salicylic acid and 5-(n-octanoyl) salicylic acid), oligopeptides and pseudodipeptides and their acylated derivatives (e.g., {2-[acetyl (3-(trifluoromethyl) phenyl) amino]-3-methylbutyrylamino}acetic acid), lipopeptides e.g. MATRIXYL 3000 available from Croda), lycopene, manganese, magnesium salts (e.g., gluconates), and combinations of any of the foregoing.

Exemplary adenosine derivatives include, but are not limited to, 2 ‘-deoxyadenosine; 2’,3′-iso-propylideneadenosine; toyocamycin, 1-methyladenosine; N-6-methyladenosine, adenosine N-oxide, 6-methyl-mercaptopurine riboside, and 6-chloropurine riboside. Other adenosine derivatives comprise adenosine receptor agonists, including phenylisopropyladenosine (“PIA”)/1-methylisoguanosine, Ns-cyclohexyladenosine (CHA), N6-cyclopentyladenosine (CPA), 2-chloro-Ns-cyclopentyl-adenosine, 2-chloroadenosine, N6-phenyladenosine, 2-phenylaminoadenosine, MECA, Ne-phenethyladenosine, 2-p-(2-carboxyethyl) phenethylamino-5′-N-ethylcarboxamido-adenosine (CGS-21680), (N-ethylcarboxamido) adenosine-S-(NECA), 5′-(N-cyclopropylcarboxamido) adenosine, DPMA (PD 129,944), and metrifudil.

In some embodiments, the composition described herein can comprise one or more adenosine derivatives that increase the intracellular concentration of adenosine, such as erythro-9-(2-hydroxy-3-nonyl) adenine (“EHNA”), iodotubercidin, or combinations thereof. Additional adenosine derivatives contemplated herein include adenosine salts and alkyl esters of adenosine.

In some embodiments, the composition can further comprise one or more pearlescent agents. Pearlescent agents are iridescent particles of any shape produced in particular by certain shellfish in their shells. Alternatively, pearlescent agents can be synthesized, i.e. man made. The pearlescent agents can be chosen from white pearlescent agents such as, but not limited to, mica covered with titanium oxide or with bismuth oxychloride, colored pearlescent agents such as, but not limited to, pearlescent agents based on bismuth oxychloride, titanium oxide-coated mica covered with iron oxides, titanium oxide-coated mica covered with in particular ferric blue or chromium oxide, or titanium oxide-coated mica covered with an organic pigment.

In some embodiments, the composition can further comprise one or more hydroxy acids. Examples of hydroxy acids include beta hydroxy acids such as salicylic acid, acetylsalicylic acid, and the like. Additional exemplary hydroxy acids suitable for use in the composition include citric acid, glycolic acid, hydroxycaproic acid, hydroxycaprylic acid, lactic acid, malic acid, tartaric acid, polyhydroxy acids including gluconolactone, and any combination thereof.

In some embodiments, the composition can further comprise one or more emulsifiers. An emulsifier keeps unlike ingredients (such as oil and water) from separating in an emulsion. Suitable emulsifiers include but are not limited to: polysorbates, laureth-4, potassium cetyl sulfate, glyceryl caprylate, and any combinations thereof.

In some embodiments, the composition can further comprise one or more antimicrobials. Suitable antimicrobials include but are not limited to: caprylyl glyceryl ether, benzalkonium chloride, benzethonium chloride, and chloroxylenol (PCMX), tea tree oil, witch hazel, rosemary oil, lemon oil, and any combination thereof.

In some embodiments, the composition can further comprise one or more humectants (water-retaining agents) to improve the level of moisture in the skin. Non-limiting examples of suitable humectants for use in the compositions described herein are described in WO 98/22085, WO 98/18444, and WO 97/01326 and include: amino acids and derivatives thereof such as proline and arginine aspartate, 1,3-butylene glycol, propylene glycol, pentylene glycol, water, codium tomentosum extract, creatinine, diglycerol, biosaccharide gum-1, glucamine salts, glucuronic acid salts, glutamic acid salts, polyethylene glycol ethers of glycerine (e. g. glycereth 20), glycerine, glycerol monopropoxylate, glycogen, hexylene glycol, honey, hydrogenated starch hydrolysates, hydrolyzed mucopolysaccharides (such as xanthan gum and biosaccharide gum-1), inositol, keratin amino acids, glycosaminoglycans, methoxy PEG 10, methyl gluceth-10 and-20, methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), propanediol, pentaerythitol, 1,2 pentanediol, PPG-1 glyceryl ether, 2-pyrrolidone-5-carboxylic acid (including salts and esters thereof), saccharide isomerate, sericin, silk amino acids, sodium acetylhyaluronate, sodium hyaluronate, sodium poly-aspartate, sodium polyglutamate, caprylyl glycol, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, and yeast extract, and mixtures thereof.

Additional humectants suitable for use herein include polyhydric alcohols selected from the group consisting of glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

In some embodiments, the composition can further comprise a gelling agent comprising a polyacrylamide-based polymer. The polyacrylamide-based polymer can, aside from being polyacrylamide itself, be a derivative thereof, and can be a mixture of a plurality of types of polymers, and can also be a copolymer with acrylamide and its derivatives as monomers. A gelling agent can be used to provide a good appearance to the skin, provide a sense of coolness to the skin, and provide a sense of refreshment without stickiness to the skin. In some embodiments, a gelling agent can include one or more pigments, or one or more fillers, including inorganic pigments, including extender pigments, coloring pigments, and whitening pigments, organic pigments, pearlescent gloss pigments, macromolecular powders, functional pigments, talc, mica, kaolin, calcium carbonate, magnesium carbonate, silicic anhydride, aluminum silicate, magnesium silicate, calcium silicate, aluminum oxide, barium sulfate, red iron oxide, yellow iron oxide, black iron oxide, chrome oxide, ultramarine blue, prussian blue, carbon black, zinc oxide, mica titanium, fish scale flakes, bismuth oxychloride, boron nitride, nylon powder, silk powder, carbomer, tar pigments, natural pigments and titanium oxide, such as amorphous or rutile type and/or anatase type crystals.

In some embodiments, the composition can comprise a pigment and/or filler that is resistant to water and an oil and can further include any conventionally used water-repellent and/or oil-repellent agent to confer water repellence and oil repellence to pigments, for example, fluorine compounds. Representative fluorine compounds which are conventionally used and can act as water-repellent and oil-repellent agents include compounds having perfluoroalkyl groups such as perfluororalkyl phosphates, perfluoroalkyl silanes, perfluoroalkyl silazanes, polyhexafluoropropylene oxides, perfluoroalkyl-group-containing organosiloxanes, per-fluoropolyethers, perfluoro alcohols, perfluoroalkylacrylate polymers, and derivatives thereof. Perfluoroalkyl phosphates can provide a uniform and stable dispersement of pigments within the formulation of a gel composition, and perfluoroalkyl silanes can have exceptional compatibility with other cosmetic ingredients. Additionally, the perfluoroalkyl phosphate-diethanol amine salt marketed by Asahi Glass as AsahiGuard AG530, and perfluoroalkyl silane coupling agents, such as LP-IT and LP-4T of Shin-Etsu Silicone, can be used.

Representative gelling agents include, but are not limited to, those marketed by Seppic under the trade names Sepigel 305, Sepigel 501, and Sepigel 600. Sepigel 305 is a mixture containing approximately 40% polyacrylamide, approximately 24%-C13-C14 isoparaffin and approximately 6% Laureth-7 (here, Laureth-7 is a non-ionic surfactant having the formula C12H25--(OCH2CH2)n-OH, wherein n has an average value of 7). Sepigel 600 is a mixture of a acrylamide/acrylamide-2-propane sulfonate copolymer, isohexadecane and polysorbate 80 (polyoxyethylene sorbitan mono-oleate (20 EO)). A suitable gelling agent comprising a polyacrylamide-based polymer is disclosed in EP 0 503 853 (Scott Bader Company Ltd.), the disclosure of which is incorporated by reference herein.

In some embodiments, the composition can further comprise hyaluronic acid (HA). In some embodiments, the HA can be in an uncrosslinked state. In some embodiments, the HA can be in a crosslinked state. Like collagen, HA is an important structural component of human tissues. Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronan present in the skin decreases.

In some embodiments, the composition can further comprise a waxy lipid, e.g., a ceramide. Ceramides help create a barrier to prevent permeability, which helps prevent dryness and irritation and can also protect the epidermis from environmental damage.

In some embodiments, the composition can further comprise at least one vitamin. In some embodiments, the composition can further comprise vitamin A or a vitamin A derivative. Examples of vitamin derivatives include, but are not limited to, retinoids such as retinal, retinoic acid, retinoate, retinyl ester, retinol, tretinoin, isotretinoin, adapalene, tazarotene, and the like. The term “retinoids” includes cis and trans derivatives of retinoids (e.g. all-trans-retinoic acid, 13-cis-retinoic acid, 13-trans retinoic acid, and 9-cis-retinoic acid).

In some embodiments, the composition can further comprise vitamin C or its derivatives, e.g., ascorbic acid, ascorbate (e.g. tetrahexyldecyl ascorbate), and the like.

In some embodiments, the composition can further comprise vitamin B, e.g., biotin, (i.e., vitamin B7), niacinamide, and the like.

In some embodiments, the composition can further comprise vitamin E, e.g. α-, β-, γ-, and σ-tocopherols and their related corresponding tocotrienols), and the like.

In some embodiments, the composition can further comprise vitamin K and derivatives thereof.

Any vitamin, vitamin analog, or derivative thereof that can be suitably formulated as a topical composition is contemplated for the present disclosure.

In some embodiments, the composition disclosed herein can further comprise one or more thickening agents. A thickening agent, i.e., structure builder, is able to suspend pigments and/or build viscosity in the composition. Thickeners and/or structure builders suitable for the present compositions include, but are not limited to, organically modified clays, fumed silica, trihydroxystearin, silicone gels or silicone elastomers, ammonium acryloyldimethyltaurate/VP copolymer, acrylates/C10-30 alkyl acrylate crosspolymer, and mixtures thereof.

Suitable organically modified clays include, but are not limited to, organically modified versions of hectorite, bentonite, smectite and montmorillonite clay (such as those sold under tradename BENTONE® from Elementis Specialties, TIXO-GEL® from Sud-Chemie, and CLAYTONE® from Southern Clay Products). Hydrophilically modified fumed silicas include, but are not limited to, WACKER HDK® N20 and T30 grades (Wacker-Chemie AG), and hydrophilic grades under tradename of AEROSIL® (Evonik). Silicone gels or silicone elastomers include, but are not limited to, the “KSG” thickening series (KSG-15, KSG-16. KSG-18, KSG-41, KSG-42, KSG-43, KSG-44) from Shin-Etsu Silicones, DOW CORNING®9040, 9041, 9045, and 9546 silicone elastomer blends from Dow Corning, SFE839™, and Velvesil™ silicone gels from Momentive Performance Materials, and WACKER-BELSIL® RG-100 from Wacker-Chemie AG.

In some embodiments, the compositions disclosed herein can further comprise one or more lipo-soluble/lipo-dispersible film-forming agent. Lipo-soluble/lipo-dispersible film-forming agents suitable for use herein include, but are not limited to, organic silicone resins (e.g., trimethylsiloxysilicate such as SRI 000 from GE Silicones) and copolymers of organic silicone resins (e.g., diisostearyl trimethylolpropane siloxy silicate such as SF1318 from GE Silicones); fluorinated silicone resins; acrylic and/or vinyl based polymers or copolymers, including silicone and/or fluorinated versions (e.g., the “KP” series of silicone acrylates from Shin-Etsu Silicones, and 3M™ Silicones “Plus” Polymer VS70 and SA70); polyurethanes (e.g., the hydroxyester triglyceride derived Poly Derm® series from Alzo International); polyesters (e.g., the Lexorez® series of polymeric polyesters from Inolex Chemical Company); and mixtures thereof.

In some embodiments, the composition disclosed herein can further comprise one or more coloring agents. Coloring agents suitable for use herein include all inorganic and organic colors/pigments, including mineral or pearl pigments suitable for use in cosmetic compositions. Such coloring agents include those either with or without a surface coating or treatment. Coloring agents can intensify coloration, and/or light scattering, and/or light reflecting effects of the composition.

In some embodiments, the composition disclosed herein can further comprise one or more sunscreens, e.g., mineral and/or physical sunscreens. Sunscreens can block UVA and/or UVB radiation. Exemplary UVA sunscreen agents include, but are not limited to, avobenzone, terephthalylidene dicamphor sulfonic acid, bis-disulizole disodium, disodium phenyl dibenzimidazole tetrasulfonate, diethylamino hydroxybenzoyl hexylbenzoate, bis-diethylamino hydroxybenzoyl benzoate, bis-benzoxazolylphenyl ethylhexylamino triazine, and combinations thereof.

Exemplary UVB sunscreen agents include, but are not limited to, octocrylene, octinoxate, octisalate, homosalate, ensulizole, ethylhexyl triazone, enzacamene, amiloxate, diethylhexyl butamido triazine, benzylidene malonate polysiloxane, padimate-O, trolamine salicylate, cinoxate, p-aminobenzoic acid and derivatives thereof, and combinations thereof.

Exemplary sunscreen agents that absorb both UVA and UVB radiation such as, for example, oxybenzone, meradimate, titanium dioxide, zinc oxide, bis-octrizole, bemotrizinol, drometrizole trisiloxane, sulisobenzone, dioxybenzone, or combinations thereof.

Specific suitable sunscreens include but are not limited to p-aminobenzoic acid, its salts and its derivatives (ethyl, isobutyl, glyceryl esters, p-dimethylaminobenzoic acid, anthranilates (i.e., o-aminobenzoates, methyl, menthyl, phenyl, benzyl, phenylethyl, linallyl, terpinyl, and cyclohexenyl esters), salicylates (amyl, phenyl, benzyl, menthyl, glyceryl, and dipropylene glycol esters), cinnamic acid derivatives (methyl and benzyl esters, alpha-phenyl cinnamonitrile, butyl cinnamoyl pyruvate), dihydroxycinnamic acid derivatives (umbelliferone, methylumbelliferone, methylaceto umbelliferone), trihydroxycinnamic acid derivatives (esculetin, methyl eSculletin, daphnetin, and the glucosides, esculin and daphnin), hydrocarbons (diphenylbutadiene, stilbene), dibenzalacetone and benzalacetophenone, naphtholsulfonates (sodium salts of 2-naphthol-3,3-disulfonic and of 2-naphthol-6,8-disulfonic acids), cihydroxynaphthoic acid and its salts, o- and p hydroxybiphenyldisulfonates, coumarin derivatives (7 hydroxy, 7-methyl, 3-phenyl), diazoles (2-acetyl-3-bromoindazole, phenyl benzoxazole, methylnaphthoxalole, various arylbenzothiazoles), quinine salts (bisulfate, sulfate, chloride, oleate, and tannate), quinoline derivatives (8-hydroxyquinoline salts, 2-phenylquinoline), hydroxy- or methoxy substituted benzophenones, uric and vilouric acids, tannic acid and its derivatives (e.g., hexaethylether), (butyl carbityl) (6-propyl piperonyl) ether, hydroquinone, benzophenones (oxybenzene, sulisobenzone, dioxybenzone, benzoresorcinol, 2,2,4,4′-tetrahydroxybenzophenone, 2,2′-dihydroxy4,4′-dimethoxybenzophenone, octabenzone, 4-isopropyhldibenzoylmethane, butylmethoxydibenzoylmethane, etocrylene, and 4-isopropyl-di-benzoylmethane, titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-bis(hydroxypropyl)aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less). In some embodiments, the composition disclosed herein can further comprise D-aspartic acid and/or D-alanine and any salts thereof. As used herein, the term “derivatives” of D-aspartic acid and D-alanine indicates D-aspartic acid and D-alanine molecules that are covalently bound to any organic group via their amino groups, carboxyl groups, or side chains, provided that the effect on promoting collagen production of D-aspartic acid and D-alanine is not impaired. Exemplary organic groups include, but are not limited to, protective groups, such as N-phenylacetyl group, and 4,4′-dimethoxytrityl (DMT) group; biopolymers, such as a protein, a peptide, a saccharide, a lipid, and a nucleic acid; synthetic polymers, such as a polystyrene, a polyethylene, a polyvinyl, a polypropylene, and a polyester; and functional groups such as an ester group. The ester group may comprise, for example, an aliphatic ester, such as methyl ester, and ethyl ester; and an aromatic ester.

In some embodiments, the composition can further comprise one or more general skin care additives such as, e.g., conditioning agents such silicones. In some embodiments, the composition can further comprise one or more shark liver oils, e.g., squalane and/or squalene. In some embodiments, the composition can further comprise one or more polysaccharides produced by microalgae, e.g., alguronic acid.

In some embodiments, the composition can further comprise at least one preservative. In some embodiments, the at least one preservative can be quartenary ammonium compounds, halogenated phenols, sorbic acid, potassium sorbate, benzoic acid, sodium benzoate, sodium citrate, sodium anisate, caprylhydroxamic acid, sodium levulinate, phenoxyethanol, or combinations thereof.

The compositions described herein can have a pH in the range of about 4 to about 8, from about 4.7 to about 5.5, from about 5 to about 7, from about 6 to about 7, from about 6.1 to about 6.8, or from about 6.4 to 6.6.

In some embodiments, the composition described herein can be a dermal filler composition, e.g., an injectable dermal filler composition. Dermal fillers are generally made of collagens and can optionally comprise hyaluronic acid (HA). Dermal filler compositions can be suitable for use on the face and body, including, e.g., around the eyes, on or around the cheeks, on or around the décolletage, on or around the hands, on or around the nails, on or around the ears, including on the earlobes, on or around the legs, and on or around the feet.

In some embodiments, the composition described herein can be used with a microneedle array, such as an array included in a sheet or patch. Microneedle arrays can comprise a plurality of microneedles that are of a length sufficient to penetrate the skin across the stratum corneum and into the viable epidermis. It some embodiments it can be desirable to deliver polypeptides to the area of epidermal/dermal junction for cosmetic or therapeutic purposes.

In some embodiments, the composition described herein can be used with microneedle sheets or patches. Microneedles and microneedle patches are suitable for delivering collagen into the epidermis and dermis of human skin on the face and body, including, e.g., the eyes, the cheeks, the lips, the décolletage, and the hands. In some embodiments, the microneedles for delivering compositions into the epidermis and dermis in a targeted manner are injectable microneedles, drug coated metal microneedles, or microneedles having dissolvable tips. Exemplary methods and disclosures regarding microneedles can be found in, for example, Aditya et al., Kinetics of collagen microneedle drug delivery system, Journal of Drug Delivery Science and Technology, vol. 52, pp. 618-623 (August 2019) and Sun et al., Transdermal Delivery of Functional Collagen Via Polyvinylpyrrolidone Microneedles, Ann. Biomed. Eng., 43(12):2978-2990 (2015), each of which is incorporated by reference in its entirety.

The compositions according to the present disclosure can comprise the recombinant collagen fragments described herein, at least one stress related protein, at least one cell wall related protein, at least one DNA or protein synthesis related protein, at least one metabolic enzyme, water, and, optionally, at least one cell integrity protein.

In some embodiments, the composition can comprise a stress related protein. In certain embodiments, the stress related protein can be an ATPase involved in protein import to the endoplasmic reticulum, an ATPase involved in protein folding, protein disulfide isomerase, Hsp90, an Hsp90 co-chaperone, thioredoxin, a cytoplasmic ATPase, a protein disulfide isomerase, or combinations thereof.

In some embodiments, the composition can comprise a cell wall related protein. In certain embodiments, the cell wall related protein can be a major exo-1,3-beta-glucanase, endo-beta-1,3-glucanase, glycosidase, 1,3-beta-glucanosyltransferase, chitin deacetylase, fructose 1,6-bisphosphate aldolase, or combinations thereof.

In some embodiments, the composition can comprise a DNA or protein synthesis protein. In certain embodiments, the DNA or protein synthesis protein can be an elongation factor 1-alpha, adenosine kinase, 60S acidic ribosomal protein P2-A, nucleoside diphosphate kinase, ribosomal protein 51, ribosomal protein L30, ribosomal protein 59, eukaryotic translation initiation factor 5A, translation initiation factor elF4A, DNA topoisomerase 2, histone methyl transferase, S-adenosyl-L-methionine-dependent tRNA, eukaryotic translation initiation factor 3 subunit 1, lysine-tRNA ligase, DNA-directed RNA polymerase subunit beta, transcription factor II, DNA ligase, and combinations thereof.

In some embodiments, the composition can comprise a metabolic enzyme. In certain embodiments, the metabolic enzyme can be an enolase I, triosephosphate isomerase, superoxide dismutase, glyceraldehyde-3-phosphate dehydrogenase, cobalamin-independent methionine synthase, 6-phosphogluconate dehydrogenase, phosphotransferase, NADPH-dependent alpha-keto amide reductase, phosphoglycerate mutase, cytoplasmic inorganic pyrophosphatase, alcohol dehydrogenase, carboxypeptidase Y inhibitor, phosphatidylglycerol/phosphatidylinositol transfer protein, cytoplasmic ATPase, mitochondrial matrix ATPase, peptidyl-prolyl cis-trans isomerase, adenosine kinase, thiol-specific peroxiredoxin, ornithine aminotransferase, ketol-acid reductoisomerse, aconitate hydratase, malate dehyrdrogenase, glycerol kinase, alanine:glyoxylate aminotransferase, nucleoside diphosphate kinase, chitin deacetylase, transketolase, NADPH oxidoreductase, fructose 1,6-bisphosphate aldolase, NADH-cytochrome b5 reductase, phosphomannomutase, mitochondrial peroxiredoxin, 3-isopropylmalate dehydrogenase, thioredoxin peroxidase, squalene epoxidase, malate dehydrogenase, coproporphyrinogen III oxidase, isocitrate dehydrogenase [NAD] subunit, catalytic subunit of NatB N-terminal acetyltransferase, carboxypeptidase, triacylglycerol lipase, adenosylhomocysteinase, flavin-containing monooxygenase, acid trehalase, high affinity methionine permease, subunit of alpha-1,6 mannosyltransferase complex, adenylate cyclase, phosphotransferase, methylenetetrahydrofolate reductase, alternative oxidase, Cu(+2)-transporting P-type ATPase, GTPase, 2-(3-amino-3carboxypropyl) histidine synthase subunit 1, peroxisome biosynthesis protein PAS1, folylpolyglutamate synthase, pyruvate kinase, serine/threonine-protein phosphatase, protein serine/threonine kinase, beta-mannosyltransferase 2, urea amidolyase, citrate synthase, lanosterol synthase, or a combination thereof.

In some embodiments, the composition can comprise from about 0.5% to about 20% by weight of at least one stress related protein, from about 0.7% to about 17% from about 1% to about 15% by weight of at least one stress related protein, from about 2% to about 13% by weight of at least one stress related protein, from about 2% to about 11% by weight of at least one stress related protein, from about 3% to about 10% by weight of at least one stress related protein, from about 4% to about 9% by weight of at least one stress related protein, from about 5% to about 8% by weight of at least one stress related protein, or from about 6% to about 7% of at least one stress related protein. In some embodiments, the composition can comprise about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of at least one stress related protein.

In some embodiments, the composition can comprise from about 0.1% to about 20% by weight of at least one cell wall related protein, from about 0.2% to about 17% by weight of at least one cell wall related protein, from about 0.3% to about 15% by weight of at least one cell wall related protein, from about 0.4% to about 13% by weight of at least one cell wall related protein, from about 0.5% to about 10% by weight of at least one cell wall related protein, from about 1% to about 9% by weight of at least one cell wall related protein, from about 1% to about 8% by weight of at least one cell wall related protein, from about 1.5% to about 7.5% by weight of at least one cell wall related protein, from about 2% to about 7% by weight of at least one cell wall related protein, from about 2.5% to about 6.5% by weight of at least one cell wall related protein, from about 3% to about 6% by weight of at least one cell wall related protein, from about 3.5% to about 5.5% by weight of at least one cell wall related protein, or from about 4% to about 5% by weight of at least one cell wall related protein. In some embodiments, the composition can comprise about 0.1%, about 0.2%, about 0.3%, about 0.4%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% by weight of at least one cell wall related protein.

In some embodiments, the composition can comprise from about 0.1% to about 20% by weight of at least one DNA or protein synthesis related protein, from about 0.2% to about 17% by weight of at least one DNA or protein synthesis related protein, from about 0.3% to about 15% by weight of at least one DNA or protein synthesis related protein, from about 0.4% to about 13% by weight of at least one DNA or protein synthesis related protein, from about 0.5% to about 10% by weight of at least one DNA or protein synthesis related protein, from about 1% to about 9.5% by weight of at least one DNA or protein synthesis related protein, from about 1.5% to about 9% by weight of at least one DNA or protein synthesis related protein, from about 1.5% to about 8.5% by weight of at least one DNA or protein synthesis related protein, from about 1.5% to about 8% by weight of at least one DNA or protein synthesis related protein, from about 2% to about 7.5% by weight of at least one DNA or protein synthesis related protein, from about 2.5% to about 7% by weight of at least one DNA or protein synthesis protein, from about 3% to about 6.5% by weight of at least one DNA or protein synthesis protein, from about 3.5% to about 6% by weight of at least one DNA or protein synthesis protein, or from about 4% to about 5.5% by weight of at least one DNA or protein synthesis protein. In some embodiments, the composition can comprise about 0.1%, about 0.2%, about 0.3%, about 0.4%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% by weight of at least one DNA or protein synthesis related protein.

In some embodiments, the composition can comprise from about 0.5% to about 35%, from about 1 to about 30% by weight of at least one metabolic enzyme, from about 5% to about 25% by weight of at least one metabolic enzyme, from about 10% to about 20% by weight of at least one metabolic enzyme, from about 12% to about 18% by weight of at least one metabolic enzyme, or from about 14% or 16% by weight of at least one metabolic enzyme. In some embodiments, the composition can comprise about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% by weight of at least one metabolic enzyme.

In some embodiments, the skincare composition can further comprise at least one emollient. In some embodiments, the composition can comprise from about 0.5% to about 50% by weight of the at least one emollient, from about 5% to about 45% by weight of at least one emollient, from about 10% to about 40% by weight of at least one emollient, from about 15% to about 35% by weight of at least one emollient, or from about 20% to about 30% of at least one emollient. In some embodiments, the composition can comprise about 0.5%, about 5%, about 10%, about 15%, about 20%, about 25%, about 30%, about 35%, about 40%, about 45%, or about 50% of the at least on emollient.

In some embodiments, the at least one emollient can be a plant or animal oil or fat, such as Cannabis seed oil, Astrocaryum yauaperyense fat, Brassica campestris seed oil, African Mango kernel oil, Prunus armeniaca kernel oil, Camelina seed oil, Argania spinosa kernel oil, Prunus armeniaca kernel oil, Hippophae rhamnoides oil, Echium plantagineum seed oil, emu oil, orange roughy oil, canola oil, Torreya nucifera seed oil, Garcinia indica Choisy seed oil, Carum carvi seed oil, Prunus armeniaca L. var anus Maxim oil, Aleurites moluccana oil, Crambe abyssinica seed oil containing erucic acid, Vaccinium macrocarpon seed oil, walnut seed oil, Ribes nigrum seed oil, Vaccinium vitis-idaea L. seed oil, rice germ oil, Pinus parviflora seed oil, Camellia sasanqua oil, Shorea seed oil, oxidized corn oil, shea oil and fat, Shorea stenoptera (seed) butler, Lupinus albus seed oil, hydrogenated avocado oil, hydrogenated olive oil, hydrogenated beef tallow, hydrogenated soybean oil, hydrogenated jojoba oil, hydrogenated vegetable oil, hydrogenated palm kernel oil, hydrogenated palm oil, castor oil hydrogenated, hydrogenated jojoba oil, hydrogenated vegetable oil, hydrogenated coconut oil, Sclerocarya birrea seed oil, Struthio camelus oil, Passiflorae incarnata L. seed oil, Camellia sinensis oil, camellia oil, Vaccinium oxycoccos seed oil, Theobroma grandiflorum seed fat, lard, creams, Coix lachryma-jobi L. var. ma-yuen Stapf Coix oil, babassu oil, peanut oil, Pistachio seed oil, Hippophae rhamnoides oil, sunflower seed oil, Vaccinium myrtillus L. seed oil, grape seed oil, partially hydrogenated perilla oil, partially hydrogenated hourse oil, prune seed oil, broccoli seed oil, Luffa cylindrica seed oil, Cucurbita pepo Linnaeus seed oil, Haematococcus pluvialis oil, Rubus chamaemorus seed oil, pine seed oil, Mangifera indica seed fat, Mangifera indica seed oil, mink oil, Melia azadirachta seed oil, peach kernel oil, begetable oil, Camellia oleifera oil, Rubus idaeus seed oil, apple seed oil, Borago officinalis seed oil, rose hip oil, seed oil of Moringa oleifera Lam., Macadamia integrifolia oil, castor oil, olive oil, almond oil, cacao oil, camellia oil, coconut oil, palm oil, tallow, jojoba oil, grape seed oil, avocado oil, safflower oil, sesame oil, Camellia sinensis oil, evening primrose oil, wheat germ oil, hazelnut oil, Limnanthes alba oil, Prumus persica kernel oil, peppermint oil, Melaleuca alternifolia oil, corn oil, canola oil, sunflower oil, linseed oil, cotton oil, soybean oil, peanut oil, rice bran oil, cacao oil, shea oil, fennel oil, perilla oil, chamomile oil, carrot oil, cucumber oil, egg yolk oil, beef fat, hourse fat, fish oil, turtle oil, orange roughy oil, or a combination thereof.

In some embodiments, the at least one emollient can be a wax, such as lacquer-tree fruit rind wax, Jasminum officinale flower wax, orange fruit rind wax, orange flower wax, hydrpogenated jojoba oil ester, carnauba wax, candelilla wax, Narcissus poeticus L. flower wax, rice bran wax, Myrica rubra f. alba fruit wax, sweet acacia flower wax, Polianthes tuberosa flower wax, beeswax, spermaceti, orange roughy oil, lanolin, rice wax, montan wax, ozokerite, or a combination thereof.

In some embodiments, the at least one emollient can be a hydrocarbon, such as isoeicosane, isododecane, isohexadecane, diethylhexylcyclohexane, pentahydrosqualane, mineral oils, petrolatum, polyisobutene, hydrogenated polyisobutene, polybutene, squalane, squalene, microcrystalline wax, ceresin wax, paraffin wax, vaseline, ozokerite, α-olefin oligomer, tetradecene, or a combination thereof.

In some embodiments, the emollient can be a natural or synthetic fatty acid, such as isomerized linoleic acid, tallowate, fatty acids (C14-28), fatty acids (C20-40), hydrogenated coconut fatty acid, palm kernel fatty acid, 10-hydroxydecanoic acid, branched fatty acids (C14-28), branched fatty acids (C21-31), behenic acid, capric acid, myristic acid, palmitic acid, stearic acid, behenic acid, lanolin fatty acid, linoleic acid, linolenic acid, lauric acid, oleic acid, isostearic acid, undecylenic acid, 1,2-hydroxystearic acid, palmitooleic acid, erucic acid, docosahexaenoic acid, eicosapentaenoic acid, isohexadecenoic acid, anteisoheneicosanoic acid, isononanoic acid, 2-ethylhexanoic acid, or a combination thereof.

In some embodiments, the at least one emollient can be a natural or synthetic higher alcohol, such as C12-16 alcohols, C14-22 alcohols, C20-22 alcohols, C20-40 alcohols, C30-50 alcohols, brassica rapeseed sterol, isocetyl alcohol, isopropanol, caprylyl glycol, rice bran sterol, hydrogenated rapeseed alcohol, cetanol, stearyl alcohol, hexyl decanol, octyl dodecanol, lauryl alcohol, capryl alcohol, myristyl alcohol, oleyl alcohol, cetostearyl alcohol, arachyl alcohol, behenyl alcohol, jojoba alcohol, chimyl alcohol, selachyl alcohol, batyl alcohol, isostearyl alcohol, cetyl alcohol, cholesterol, phytosterol, lanolin alcohol, hydrogenated lanolin alcohol, palmityl alcohol, 2-decyltetradecinol, or a combination thereof.

In some embodiments, the at least one emollient can be an ester, such as isopropyl C12-15-pareth-9 carboxylate, octyldodecyl pyrrolidone carboxylic acid, menthyl pyrrolidone carboxylic acid, lauryl pyrrolidone carboxylic acid, PEG-2 isosedes-7 carboxylic acid cetyl ester, di-PPG-3 myristyl ether adipate, di-PPG-2 myreth-10 adipate, diisopropyl adipate, diheptylundecyl adipate, ethyl avocadate, butyl avocadate, methyl gluceth-20 benzoate, ethyl isostearate, (isostearic acid/succinic acid) castor oil, hydrogenated castor oil isostearate, batyl isostearate, hexyldecyl isostearate, (isostearic acid/bees wax/succinic acid) castor oil, isodecyl isononanoate, isotridecyl isononanoate, isononyl isononanoate, octyl isononanoate, cetearyl isononanoate, sucrose acetate isobutyrate, caprylyl eicosenoate, butyl ethyl propanediol ethylhexanoate, octyldodecyl erucate, oleyl erucate, alkyl octanoate (C14, C16, and C18), (octanoic acid/stearic acid/adipic acid) glyceryl, cetyl octanoate, stearyl octanoate, cetearyl ethylhexanoate, hexyldecyl ethylhexanoate, ethyl olivate, oleyl oleate, caprylic/capric glycerides, caprylic/capric/coconut fatty acid glyceride, caprylic/capric/succinic triglyceride, cetyl caprate, triisostearyl citrate, triisooctyldodecyl citrate, citric/lactic/linoleic/oleic tetraglyceride, isostearyldiglyceryl succinate, diethoxylethyl succinate, dioctyl succinate, phytosteryl rice bran oil fatty acid, cetyl acetate, farnesyl acetate, lanolin alcohol acetate, linalyl acetate, propylene glycol diisostearate, dimer-dilinoleyl diisostearate, dimer-dilinoleyl diisostearate, neopentyl glycol diisostearate, neopentyl glycol diethylhexanoate, PEG-18 castor oil dioleate, di-(caprylic acid/capric acid) butylene glycol, isosorbide dicaprylate, di-(caprylic acid/capric acid) polyethylene glycol, dicocoyl pentaerythrityl distearyl citrate, polyethylene glycol distearate, PG-20 methylglucose distearate, pentaerythrityl distearate, diethyl pentanediol dineopentanoate, methyl pentanediol dineopentanoate, polyethylene glycol dinonanoate, C10-30 cholesterol/lanosterol esters, C12- to C-18 cetyl esters, glycol dilaurate, diisopropyl dimer dilinoleate, pentaerythrityl hydrogenated rodinate, methyl hydrogenated rosinate, glyceryl diisostearate/hydrogenated rosinate, ethylhexyl stearate, isocetyl stearoyl oxystearate, octyldodecyl stearoyl oxystearate, dibutyloctyl sebacate, octyldodecyl/PPG-3 myristyl ether dimer dilinoreate, di-(isostearyl/phytophytoryl) dimer dilinoleate, dicetearyl dimer dilinoleate, hydrogenated castor oil dimer dilinoleate, stearyl/PPG-3 myristyl ether dimer dilinoleate, dimer dilinoleyl dimer dilinoleate, bis-behenyl/isostearyl/phytosteryl dimer dilinoleyl dimer dilinoleate, phyto phytosteryl/isostearyl/cetyl/stearyl/behenyl dimer dilinoleate, dimer dilinoleyl hydrogenated rosin condensate, C14-15 dialkyl carbonates, diethylhexyl carbonate, dicaprylyl carbonate, pentaerythrityl tetraisostearate, pentaerythrityl tetraoctanoate, polyglyceryl-10 dodecacaprylate/caprate, diisocetyl dodecanedioate, dioctyldodecyl dodecanedioate, triisostearate, erythrityl triethylhexanoate, ditrimethylolpropane triethylhexanoate, triethylhexanoin (trioctanoin), trimethylolpropane tiethylhexylhexanoate trimethylolpropane, tricaprylin, caprylic/capric triglyceride, caprylic/capric/stearic triglyceride, caprylic/capric/myristic/stearic triglyceride, C8-12 acid-triglycerides, tristearin, tripalmitin, tripalmitolein, trihydroxystearin, tribehenate, dipentaerythrityl tri-polyhydroxystearate, trimyristin, C12-15 alkyl lactates, ethyl lactate, octyldodecyl lactate, butyl lactate, menthyl lactate, isodecyl neopentanoate, myristyl neopentanoate, nonanoic acid octyl ester, nonanoic acid cholesterol ester, ethylhexyl palmitate, cetyl palmitate, bis-diglyceryl polyacyladipate-1, bis-diglyceryl polyacyladipate-2, dipentaerythrityl pentahydroxystearate/pentaisostearate, ethylhexyl dihydroxystearate, octyl hydroxystearate, hydrogenated castor oil hydroxystearate, dipentaerythrityl hexahyroxystearate/stearate/rosinate, phytosteryl hydroxystearate, PPG-6 castorate, methyl ricinoleate, myristyl propionate, C10-40 isoalkyl acid cholesterol esters, dipentaerythrityl hexahydroxystearate, dipentaerythrityl hexahydroxystearate/hexastearate/hexarosinate, stearyl heptanoate, glyceryl behenate/eicosadioate, jojoba esters, isopropyl jojobate, ethyl macadamiate, cholesteryl macadamiate, phytosteryl macadamiate, dioctyl maleate, diethylhexyl syringylidenemalonate, heyldecyl myristoyl methylaminopropionate, citronellyl methylcrotonate, medowfoam estolide, octyldodecyl meadowfoamate, glyceryl cocoate/citrate/lactate, decyl cocoate, cholesteryl butyrate, dihydrocholesteryl butyrate, octyldodecyl lanolate, cholesteryl lanolate, octyldodecyl ricinoleate, diethylhexyl malate, trioleyl phosphate, tricetyl phosphate, glyceryl tri-2-ethylhexanoate, cetyl 2-ethylhexanoate, neopentyl glycol di-2-ethylhexanoatte, isopropyl myristate, butyl myristate, isopropyl palmitate, ethyl stearate, octyl palmitate, isocetyl isostearate, butyl stearate, ethyl linoleate, isopropyl linoleate, ethyl oleate, isocetyl myristate, isostearyl myristate, isostearyl palmitate, octyldodecyl myristate, diethyl sebacate, diisopropyl adipate, isoalkyl neopentanoate, caprylic/capric triglyceride, trimethylolpropane tri-2-ethylhexanoate, trimethylolpropane triisostearate, pentaerythritol tetra-2-ethylhexanoate, cetyl caprylate, decyl laurate, hexyl laurate, decyl myristate, myristyl myristate, cetyl myristate, glyceryl trimyristate, stearyl stearate, decyl oleate, cetyl ricinoleate, isostearyl laurate, isotridecyl myristate, isocetyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol diloleate, propylene glycol dioleate, propylene glycol dicaprate, propylene glycol dicaprylate/dicaprate, caprylic/capric triglyceride, propylene glycol dicaprylate, neopentyl glycol dicaprate, neopentyl glycol dioctanoate, glyceryl tricaprylate, glyceryl triundecylate, glyceryl triisopalmitate, glyceryl triisostearate, octyldodecyl neodecanoate, octyldodecyl neopentanoate, isostearyl octanoate, hexyldecyl neodecane, hexyldecyl neodecanoate, octyldodecyl neodecanoate, isostearyl isostearate, octyldecyl isostearate, polyglyceryl oleate ester, polyglyceryl isostearate ester, dipropyl carbonate, C12- to C18-dialkyl-carbonates, triisocetyl citrate, triisoarachidyl citrate, triisooctyl citrate, lauryl lactate, myristyl lactate, cetyl lactate, octyldecyl lactate, lanolin lactate, triethyl citrate, acetyltriethyl citrate, acetyltributyl citrate, trioctyl citrate, diisostearyl malate, 2-ethylhexyl hydroxystearate, 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, hexyldecyl dimethyloctanoate, and isopropyl lanolate; lanolins such as liquid lanolin, hydrogenated lanolin, adsorption refined lanolin, lanolin acetate, acetylated lanolin, hydroxylanolin, polyoxyethylene lanolin, hard lanolin fatty acid, and cetyl·lanolyl acetate; sphingophospholipids such as lecithin, phosphatidylcholine, phosphatidylethanolamine, phosphatidylserine, phosphatidylglycerol, phosphatidylinositol, sphingomyelin, or a combination thereof.

In some embodiments, the at least one emollient can be a phospholipid, such as phosphatidic acid and lysolecithin; phospholipid derivatives such as hydrogenated soybean phospholipid, partially hydrogenated soybean phospholipid, hydrogenated egg yolk-phospholipid, and partially hydrogenated egg yolk-phospholipids; sterols such as dihydrocholesterol, lanosterol, dihydrolanosterol, and cholic acid; silicon-containing oil agents such as methylphenyl polysiloxane, methylhydrogen polysiloxane, octamethylcyclotetrasiloxane, stearoxysilicone, decamethylcyclopentasiloxane, or dodecamethyl cyclohexasiloxane; sapogenins; saponins; and fluorine-containing oil agents such as perfluoropolyether, perfluorodecalin, perfluorooctane, or a combination thereof.

In some embodiments, the at least one emollient can be cocoglycerides, cetyl alcohol, tetradecane, carthamus tinctorius (safflower) oleosomes, or combinations thereof.

In some embodiments, the composition can further comprise at least one emulsifier. In some embodiments, the composition can comprise from about 1% to about 15% by weight of the at least one emulsifier, from about 2% to about 10% by weight of the at least one emulsifier, from about 3% to about 9% by weight of the at least one emulsifier, from about 4% to about 8% by weight of the at least one emulsifier, or from about 5% to about 7% by weight of the at least one emulsifier. In some embodiments, the composition can comprise about 1%, about 2%, about 3%, about 4%, about 5%, about 6%, about 7%, about 8%, about 9%, about 10%, about 11%, about 12%, about 13%, about 14%, or about 15% by weight of the at least one emulsifier.

In some embodiments, the at least one emulsifier can be a cationic, anionic, or nonionic emulsifiers. In some embodiments, the at least one emulsifier is can be glyceryl stearate, glyceryl monooleate, PEG stearates (such as, but not limited to, PEG-100 stearate, PEG-200 stearate, PEG-300 stearate, etc.), sorbitan sesquioleate, sorbitan olivate, sorbitan stearate, lecithin, undeceth-3, PEG-20 methyl glucose sesquisterate, trideceth-3, trideceth-12, laureth-9, behenoyl stearic acid, oleth-2, oleth-20, sorbitan laurate, sorbitan palmitate, sorbitan oleate, sorbitan trioleate, steareth-2, steareth-20, steareth-21, laureth-23, C11-15-Pareth-15, PPG-24-buteth-27, Avena sativa (oat) peptides, high molecular weight polymers of ethylene oxide and propylene oxide, PPG5-ceteth-10 phosphate, oleth-5 phosphate, dioleyl phosphate, oleth-3 phosphate, oleth-10 phosphate, lauryl phosphate, trideceth-3 phosphate, trideceth-6 phosphate, deceth-6 phosphate, trilaureth-4 phosphate, C20-22 alcohols, polyglycerol-10 decaoleate, polyglyceryl-3 oleate, PEG/PPG-20/6 dimethicone, bis-PEG/PPG-20/20 dimethicone, bis-PEG/PPG-16/16 PEG/PPG-16/16 dimethicone, bis-PEG/PPG20/5 PEG/PPG-20/5 dimethicone, methoxy PEG/PPG-25/4 dimethicone, bis-PEG/PPG-14/14 dimethicone, PEG-11 methyl ether dimethicone, PEG/PPG-20/22 butyl ether dimethicone, lauryl PEG-9 polydimethylsiloxyethyl dimethicone, PEG-10 dimethicone, polyglyceryl-3 disiloxane dimethicone, lauryl polyglyceryl-3 polydimethylsiloxyethyl dimethicone, lauryl PEG-8 dimethicone, sodium stearate, sucrose laurate, sucrose myristate, sucrose stearate, methyl glucose sesquistearate, or combinations thereof.

In some embodiments, the at least one emulsifier can be xantham gum, acacia gum, candelilla polyglyceryl-3 esters, jojoba polyglyceryl-3 esters, rice bran polyglyceryl-3 esters, glyceryl stearate, cetearyl alcohol, sodium stearoyl lactylate, or combinations thereof.

In some embodiments, the composition can further comprise from about 0.1% to about 10% by weight of at least one antioxidant, from about 0.1% to about 9% by weight of at least one antioxidant, from about 0.5% to about 8.5% by weight of at least one antioxidant, from about 1% to about 7.5% of at least one antioxidant, from about 1.5% to about 7% of at least one antioxidant, from about 2% to about 6.5% of at least one antioxidant, from about 2.5% to about 6% of at least one antioxidant, from about 3% to about 5.5% of at least one antioxidant, or from about 3.5% to about 5% of at least one antioxidant. In some embodiments, the skincare composition can comprise about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% of the at least one antioxidant.

In some embodiments, the at least one antioxidant can be vitamin A and derivatives and salts thereof such as retinol, dehydroretinol, retinol acetate, retinol palmitate, retinal, retinoic acid, and vitamin A oil; vitamin B and derivatives and salts thereof such as pyridoxine, pyridoxal, pyridoxal-5-phosphate, and pyridoxamine; vitamin D and derivatives and salts thereof such as ergocalciferol, cholecalciferol, and 1,2,5-dihydroxy cholecalciferol; or vitamin E and derivatives and salts thereof such as α-tocopherol, β-tocopherol, γ-tocopherol, 6-tocopherol, α-tocotrienol, β-tocotrienol, γ-tocotrienol, 6-tocotrienol, tocopherol acetate, and tocopherol nicotinate; or a combination thereof. In some embodiments, the at least one antioxidant can be trolox and salts thereof.

In some embodiments, the at least one antioxidant can be carotenoids and derivatives thereof such as α-carotene, β-carotene, γ-carotene, cryptoxanthin, astaxanthin, fucoxanthin, or a combination thereof.

In some embodiments, the at least one antioxidant can be dihydroxytoluene, butylhydroxytoluene, butylhydroxyanisole, dibutylhydroxytoluene, α-lipoic acid, dehydrolipoic acid, glutathione, and their derivatives and salts; uric acid; erythorbic acid and its derivatives and salts such as erythorbic acid and sodium erythorbate; gallic acid and its derivatives and salts such as gallic acid and propyl gallate; rutin and its derivatives and salts such as rutin and α-glycosyl rutin; triptophan and its derivative and salts; histidine and its derivatives and salts; cysteine and its derivatives and salts such as N-acetylcysteine, N-acetylhomocysteine, N-octanoyl cysteine, and N-acetylcysteine methyl ester; cystine and its derivatives and salts such as N,N′-diacetylcystine dimethyl ester, N,N′-dioctanoylcystine dimethyl ester, and N,N′-dioctanoylhomocystine dimethyl ester; carnosine and its derivatives and salts; homocarnosine and its derivatives and salts; anserine and its derivatives and salts; carcinine and its derivatives and salts; dipeptides or tripeptide derivatives and their salts containing histidine and/or triptophan and/or histamine; flavonoids such as flavanone, flavone, anthocyanin, anthocyanidin, flavonol, quercetin, quercitrin, myricetin, fisetin, hamamelitannin, catechin, epicatechin, gallocatechin, epigallocatechin, epicatechin gallate, and epigallocatechin gallate; tannic acid, caffeic acid, ferulic acid, protocatechuic acid, chalkone, orizanol, carnosol, sesamol, sesamin, sesamolin, zingerone, curcumin, tetrahydrocurcumin, clovamide (caffeoyldopa), deoxyclovamide, shogaol, capsaicin, vanillamide, ellagic acid, bromphenol, flavoglaucin, melanoidin, riboflavin, riboflavin butyrate, riboflavin mononucleotide, flavin adenine dinucleotide, ubiquinone, ubiquinol, mannitol, bilirubin, cholesterol, ebselen, selenomethionine, ceruloplasmin, transferrin, lactoferrin, albumin, superoxide dismutase, catalase, glutathione peroxidase, metallothionein, thioredoxin, thiotaurine, 0-phosphono pyridoxyliden rhodamine, and N-(2-hyroxybenzyl)amino acid, and their derivatives and salts; N-(4-pyridoxylmethylene)amino acid, and its derivatives and salts; and others such as lysine, 1-methionine, proline, silymarin, tea extract, grape bark/seed extract, melanin, rosemary extract, or a combination thereof.

In some embodiments, the at least one antioxidant can be vitamin E, vitamin A, vitamin B, or vitamin D.

In some embodiments, the composition can further comprise from about 0.1% to about 10% by weight of at least one humectant, from about 0.5% to about 10% of at least one humectant, from about 0.5% to about 9% of at least one humectant, from about 0.5% to about 8% of at least one humectant, from about 0.5% to about 7% of at least one humectant, from about 0.5% to about 6% by weight of at least one humectant, 0.5% to about 5% by weight of at least one humectant, from about 1% to about 10% by weight of at least one humectant, from about 2% to about 9% by weight of at least one humectant, from about 3% to about 8% by weight of at least one humectant, from about 4% to about 7% by weight of at least one humectant, or from about 5% to about 6% of at least one humectant. In some embodiments, the composition can comprise about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of at least one humectant.

In some embodiments, the at least one humectant can be glycerol, sorbitol, alkylene glycols (e.g., propylene glycol, dipropylene glycol, polypropylene glycol, polyethylene glycol, hexylene glycol, butylene glycol, 1,3-butylene glycol, etc.), propanediol, capryly glycol, ethylhexylglycerin, C₁₋₃ alkoxylated glucose derivatives, glyceryl triacetate, hexanetriol, vinyl alcohol, xylitol, maltitol, polydextrose, alkoxylated glycerin (e.g., ethoxylated glycerin), quillaia, urea, aloe vera gel, MP Diol (also known as 2-methyl-1,3-propane diol), alpha hydroxyl acids (e.g., lactic acid), honey, or combinations thereof.

In some embodiments, the at least one humectant can be propanediol, caprylyl glycol, ethylhexylglycerin, or combinations thereof.

Chelating agents bind with metal ions and prevent them from chemically reacting with other substances in a formulation. In some embodiments, the skincare composition can further comprise from about 0.1% to about 5% by weight of at least one chelating agent, from about 0.5% to about 4% by weight of at least one chelating agent, from about 0.5% to about 3% by weight of at least one chelating agent, from about 0.5% to about 2.5% by weight of at least one chelating agent, from about 1% to about 5% by weight of at least one chelating agent, from about 1.5% to about 3.5% by weight of at least one chelating agent, or from about 2% to about 3% by weight of at least one chelating agent. In some embodiments, the skincare composition can comprise about 0.1%, about 0.5%, about 1%, about 1.5%, about 2%, about 2.5%, about 3%, about 3.5%, about 4%, about 4.5%, or about 5% by weight of at least one chelating agent.

In some embodiments the at least one chelating agents can be ethylenediaminetetraacetates such as ethylenediaminetetraacetate (EDTA), EDTA disodium salt, EDTA trisodium salt, and EDTA tetrasodium salt hydroxyethylethylenediamine triacetates such as the sodium salt of hydroxyethylethylenediaminetriacetates; trisodium ethylenediamine disuccinate; phosphonic acid and sodium salts thereof, or the like, such as tripolyphosphoric acid, diethylenetriaminepentaacetates, phytic acid, and etidronic acid; poly (amino acids) such as sodium oxalate, polyaspartic acid, and polyglutamic acid; and others such as sodium polyphosphate, sodium metaphosphate, phosphoric acid, pyrophosphate, hexametaphosphate, sodium phytate, sodium citrate, citric acid, alanine, dihydroxyethyl glycine, gluconic acid, succinic acid, tartaric acid, or a combination thereof.

In some embodiments, the at least one chelating agent can be trisodium ethylenediamine disuccinate, sodium phytate, or combinations thereof.

In some embodiments, the composition can further comprise from about 0.5% to about 10% by weight of at least one whitening agent, from about 1% to about 10% by weight of at least one whitening agent, from about 2% to about 9% by weight of at least one whitening agent, from about 3% to about 8% by weight of at least one whitening agent, from about 4% to about 7% by weight of at least one whitening agent, from about 5% to about 6% bu weight of at least one whitening agent. In some embodiments, the skin care composition can comprise from about 2% to about 5% by weight of at least one whitening agent. In some embodiments, the skincare composition can comprise about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of at least one whitening agent.

In some embodiments, the at least one whitening agent can be an alkoxysalicylic acid and salts thereof such as 3-methoxysalicylic acid, 3-ethoxysalicylic acid, 4-methoxysalicylic acid, 4-ethoxysalicylic acid, 4-propoxysalicylic acid, 4-isopropoxysalicylic acid, 4-buthoxysalicylic acid, 5-methoxysalicylic acid, 5-ethoxysalicylic acid, 5-propoxysalicylic acid, or a combination thereof.

In some embodiments, the at least one whitening agent can be hydroquinone and derivatives thereof, such as arbutin, α-arbutin, hydroquinone α-L-glucose, hydroquinone β-L-glucose, hydroquinone α-D-galactose, hydroquinone β-D-galactose, hydroquinone α-L-galactose, hydroquinone β-L-galactose, hydroquinone α-D-ribose, hydroquinone β-D-ribose, hydroquinone α-L-ribose, hydroquinone β-L-ribose, hydroquinone α-D-arabinose, hydroquinone β-D-arabinose, hydroquinone α-L-arabinose, hydroquinone β-L-arabinose, hydroquinone α-D-glucosamine, hydroquinone β-D-glucosamine, hydroquinone α-L-glucosamine, hydroquinone β-L-glucosamine, hydroquinone α-D-galactosamine, hydroquinone β-D-galactosamine, hydroquinone α-L-galactosamine, hydroquinone β-L-galactosamine, hydroquinone α-D-glucuronic acid, hydroquinone β-D-glucuronic acid, hydroquinone α-L-glucuronic acid, hydroquinone β-L-glucuronic acid, hydroquinone α-D-galacturonic acid, hydroquinone β-D-galacturonic acid, hydroquinone α-L-galacturonic acid, hydroquinone β-L-galacturonic acid, or a combination thereof.

In some embodiments, the at least one whitening agent can be ranexamic acid and its derivatives and salts; resorcin and its derivatives such as 4-n-butylresorcinol; kojic acid and its derivatives and salts; ellagic and linoleic acids and their salts, phenyl mercury hexachlorophene, mercuric oxide, mercurychloride, hydrogen peroxide, zinc peroxide, 2-aminophenol and derivatives thereof, ferulic acid and derivatives thereof, placenta extract, glutathion, orizanol, butylresorcinol, or a combination thereof.

In some embodiments, the at least one whitening agent can be from plant extracts, such as Pimpinella anisum extract, Japanese knotweed extract, Daphne pseudomezereum extract, Senna obtusifolia extract, seed extract of Cassia obtusifolia L., root extract of Astsagalusu mambranaceus Bge, Astsagalusu mambranaceus Bge extract, Trichosanthes laceribracteata extract, Xanthium strumarium extract, Gastrodia data extract, Pyracantha fortuneana fruit extract, Polygonum sachalinense Fr. Schm extract, Lindera strychnifolia F. Vill extract, Cucurbita (pumpkin) extract, Typha latifolia L. extract, Euphorbia kansui Liou extract, Agrimonia pilosa Ledeb. var. japonica (Miq.) Nakai extract, Lindera umbellata extract, Saxifraga fusca var. kikubuki extract, Agave sisalana extract, Clematis chinensis extract, Clematis chinensis Osbeck extract, Cerasus speciosa (Koidz.) H. Ohba extract, Cerasus sargentii (Rehder) H. Ohba extract, Prunus incisa Thunb extract, Prunus nipponica extract, Cerasus subhirtella (Miq.) S. Y. Sokolov extract, Prunus lannesiana Wilson extract, Aster tataricus extract, Trachycarpus fortunei extract, Iris florentina L. extract, Clematis terniflora extract, Magnolia salicifolia extract, Saxifraga fortunei var. incisolobata extract, Oenothera tetraptera extract, dried seed extract of Convolvulaceael cuscuta chinensis Lam./C. japonica Choisy, Cuscuta australis extract, Cuscuta japonica Choisy extract, Artemisia absinthium extract, Achillea alpina L. extract, Dictamnus albus extract, Anethum gra veolens L. extract, Reynoutria japonica var. terminalis extract, Tribulus terrestris extract, Pyrrosia lingua (Thunb.) Farw extract, Typha domingensis extract, root extract of Angelica dahurica, floral blanca extract, hamula (Brickellia cabanillesy) extract, Artemisia fukudo Makino extract, Convolvulus arvensis extract, Santalum album extract, Ganoderma lucidum extract, Leonurus japonicus Houtt extract, Salix gilgiana Seemen extract, Salix chaenomeloides Kimura extract, Salix gracilistyla extract, Salix Integra extract, Salix kinuyanagi Kimura extract, Salix koriyanagi Kimura extract, Salix matsudana Koidz. var. tortuosa Vilm extract, Salix reinii Fr. et Say extract, Salix sieboldiana extract, Toisusu urbaniana Kimura extract, Salix viminalis/Salix longifolia extract, Salix vulpina Anderss extract, Populus maximowiczii A. Henry extract, dried bark extract of Myrica rubra, Agave americana var. marginata extract, Agave americana extract, Agave americana var. marginata extract, Edgeworthia papyrifera Sieb. et Zucc extract, Enteromorpha sp. extract, Enteromorpha linza/compressa extract, Enteromorpha prolifera extract, Enteromorpha compressa (Linnaeus) Nees extract, Ulva intestinalis Linnaeus extract, Ulva Linnaeus extract, Laminaria extract, Laminaria Japonica Areschoug extract, Laminaria ochotensis extract, Laminaria religiosa Miyabe extract, Laminaria angustata extract, Undaria pinnatifida extract, Undaria undarioides extract, Undaria peterseniana extract, Sargassum fusiforme extract, Fucus evanescens C. Agardh (F. vesiculosus) extract, Padina arborescens extract, Padina australis Hauck extract, Padina australis Hauck var. cuneata Tak. Tanaka et K. Nozawa extract, Padina boryana Thivy extract, Padina crassa Yamada extract, Padina japonica Yamada extract, Padina minor Yamada extract, Padina stipitata Tak. Tanaka et K. Nozawa extract, Eucheuma serra (J. Agardh) J. Agardh extract, Eucheuma amakusaense Okamura extract, Eucheuma denticulatum (Burman) Collins et Hervey extract, Eucheuma arnoldii Weber-van Bosse extract, Chondrus ocelatus Holmes extract, Chondrus verrucosus Mikami extract, Chondrus nipponicus Yendo extract, Chondrus pinnulatus (Harvey) Okamura extract, Gigartinales Schmitz extract, Chondracanthus teedii extract, Chondracanthus intermedius extract, Dictyopteris latiuscula extract, Dictyopteris polypodioides extract, Sphaerotrichia divaricata (C. agardh) Kylin extract, Cymathaere extract, Cymathaere japonica extract, Sargassum hemiphyllum Agardh extract, Sargassum segii extract, Sargassum filicinum extract, Sargassum sagamianum extract, Sargassum nigrifolium extract, Sargassum piluliferum (Turner) C. Agardh extract, Sargassum tosaense Yendo extract, Sargassum patens C. Agardh extract, Sargassum thunbergii (Mertens ex Roth) Kuntze extract, Sargassum ringgoldianum Harvey ssp. Ringgoldianum extract, Grateloupia filicina (Lamouroux) C. Agardh extract, Halymenia agardhii extract, Sebdenia polydactyla extract, Grateloupia acuminata Holmes extract, Polyopes affinis (Harvey) Kawaguchi et Wang extract, Gracilaria gagas extract, Ceratodictyon spongiosum Zanardini extract, Lomentaria catenata Harvey extract, Lomentaria pinnata Segawa extract, Lomentaria pinnata Segawa extract, Laurencia intermedia Yamada extract, Laurencia undulata extract, Laurencia pinnata Yamada extract, Laurencia brongniartii J. Agardh extract, Odonthalia corymbifera (Gmelin) Greville extract, Tilia extract, Camotede azafran extract, Hibiscus spp. extract, Poleoverde verde extract, Raphanus sativus extract, Sargassum confusum C. Agardh extract, Sargassum kjellmanianum Yendo extract, Sargassum macrocarpum C. Agardh extract, Perilla frutescens var. crispa extract, placenta extract, “RUSINOL” (4-n-butylresorcinol), silk extract, Acacia Mill extract, Malpighia glabra extract, Abutilon avicennae extract, Betula pendula extract, Quercus (gallnut) extract, Castanea crenata extract, Isodon umbrosus var. leucanthus f. kameba extract, Isodon trichocarpus extract, Rabdosia japonica (Burm. fil.) H. Hara extract, Oenanthe javanica extract, Fagopyrum esculentum extract, Durvillaea antarctica extract, Capsella bursa-pastoris extract, Eupatorium japonicum extract, Moraceae extract, Gardenia jasminoides extract, Angelica acutiloba extract, Sanguisorba officinalis extract, Sophora flavescens extract, Artemisia indica var. maximowiczii extract, Lonicera japonica extract, Phellodendron amurense extract, Houttuynia cordata extract, Poria cocos extract, Coix lacryma-jobi var. ma Yuen extract, Lamium album L. var. barbatum extract, Humulus lupulus extract, Crataegus cuneata extract, Eucalyptus extract, Achillea millefolium extract, Althaea extract, Cinnamomum loureiri Nees extract, Vitex rotundifolia L. fil. extract, Hamamelis virginiana extract, Morus australis extract, Platycodon grandiflorum extract, Euphorbia lathyris L. extract, Iris japonica extract, Ephedra intermedia extract, Cnidium officinale Makino extract, Aralia cordata extract, Bupleurum/Eryngium extract, Saposhnikovia divaricata extract, Glehnia littoralis extract, Scutellaria baicalensis extract, peony root bark extract, Paeonia lactiflora Paeonia extract, Geranium thunbergii extract, Pueraria lobata extract, Glycyrrhiza uralensis root extract, Rhus javanica extract, Aloe barbadensis Miller extract Cimicifugae rhizoma extract, Carthamus tinctorius extract, green tea extract, red tea extract, Uncaria gambir extract, Matricaria recutita extract, Persicaria tinctoria extract, oil-soluble Glycyrrhiza glabra (licorice) root extract, Cacumen Tamaricis extract, Saxifraga stolonifera extract, or a combination thereof.

In some embodiments, the at least one whitening agent can be hydroquinone, kojic acid, or combinations thereof.

In some embodiments, the composition can further comprise from about 35% to about 75% by weight of at least one surfactant. In some embodiments, the composition can comprise from about 25% to about 80% by weight of at least one surfactant, from about 30% to about 75% by weight of at least one surfactant, from about 35% to about 70% by weight of at least one surfactant, from about 40% to about 65% by weight of at least one surfactant, or from about 50% to about 60% by weight of at least one surfactant. In some embodiments, the composition can comprise about 25%, about 30%, about 35%, about 40%, about 45%, about 50%, about 55%, about 60%, about 65%, about 70%, about 72%, about 75%, or about 80% by weight of at least one surfactant.

In some embodiments, the at least one surfactant can be a natural surfactant, such as glyceryl stearate citrate, potassium cetyl phosphate, hydrogenated palm glycerides, glyceryl oleate citrate, caprylic/capric triglyceride, lauryl glucoside, decyl glucoside, caprylyl/capryl glucoside, coco-glucoside, glyceryl oleate, sodium cocoyl glutamate, disodium cocoyl glutamate, sodium stearoyl glutamate, sodium lauroyl glutamate, sodium myristoyl glutamate, coco-betaine, cocamidopropyl betaine, disodium cocoamphodiacetate, caprylyl capryl wheat bran/straw glycosides, apricot kernel oil (pressed or refined), Argania spinosa kernel oil, sorbitan stearate, sorbityl laurate, Persea gratissima oil, glyceryl stearate citrate, sodium arganamphoacetate, sodium babassuamphoacetate, sodium cocoabutteramphoacetate, sodium cottonseedamphoacetate, sodium sheabutteramphoacetate, sodium mangoamphoacetate, sodium olivamphoacetate, sodium ricebranamphoacetate, sodium sweetalmondamphoacetate, sodium sunflowerseedamphoacetate, coco-caprylate, cetyl alcohol, stearyl alcohol, glyceryl oleate, Theobroma cacao seed butter, potassium cocoate, sodium stearoyl lactylate, acacia senegal gum, hydrolyzed rhizobian gum, polyglyceryl-10 laurate, polyglyceryl-3 cocoate, polyglyceryl-3 polyricinoleate, polyglyceryl-3 ricinoleate, brasissyl isoleucinate esylate, brassica alcohol, polyglyceryl-3 sorbityl linseedate, Olea europaea fruit oil, cocoyl methyl glucamide, sodium lauroyl oat amino acids, Chenopodium quinoa seed extract, coconut alcohol, polyglyceryl-10 decaoleate, polyglyceryl-10 laurate, polyglyceryl-10 myristate, polyglyceryl-10 oleate, polyglyceryl-10 palmitate, polyglyceryl-10 stearate, polyglyceryl-10 distearate, polyglyceryl-10 tristearate, polyglyceryl-10 pentaoleate, polyglyceryl-10 pentastearate, polyglyceryl-10 heptahydroxystearate, heptyl glucoside, Sesamum indicum seed oil, sweet almond oil polyglyceryl-6 esters, apricot kernel oil polyglyceryl-10 esters, apricot kernel oil polyglyceryl-6 esters, argan oil polyglyceryl-6 esters, babassu oil polyglyceryl-6 esters, Adansonia digitata seed oil polyglyceryl-6 esters, coconut oil polyglyceryl-6 esters, watermelon seed oil polyglyceryl-6 esters, Trichilia emetica seed oil polyglyceryl-6 esters, Sclerocarya birrea oil polyglyceryl-6 esters, Schinziophyton rautanenii seed oil polyglyceryl-6 esters, monoi oil polyglyceryl-6 esters, oleic sunflower oil polyglyceryl-10 esters, oleic sunflower oil polyglyceryl-6 esters, olive oil polyglyceryl-6 esters, safflower seed oil polyglyceryl-6 esters, shea butter polyglyceryl-6 esters, Ximenia americana seed oil polyglyceryl-6 esters, sorbitan palmitate, sunfloweroyl methylglucamide, sodium laurylglucosides hydroxypropylsulfonate, cocamidopropyl hydroxysultaine, or a combination thereof.

In some embodiments, the at least one surfactant can be sorbitan monolaurate, coconut oil fatty acid sorbitan, sorbitan distearate, sorbitan monostearate, sorbitan tristearate, sorbitan monoisostearate, sorbitan monooleate, sorbitan trioleate, sorbitan fatty acid ester, sorbitan monopalmitate, sorbitan sesquioleate, sorbitan sesquistearate, polyoxyethylene sorbitan monolaurate, polyoxyethylene sorbitan monostearate, ethylene glycol monostearate, ethylene glycol distearate, diethylene glycol distearate, ethylene glycol fatty acid ester, glyceride monostearate, glyceride distearate, glyceryl isostearate, polyoxyethylene glyceryl isostearate, polyoxyethylene glyceryl triisostearate, diglyceryl isostearate, glyceryl diisostearate, glyceryl triisostearate, diglyceryl triisostearate, propylene glycol monostearate, self-emulsifying propylene glycol stearate, glyceryl ricinoleate, propylene glycol ricinoleate, propylene glycol fatty acid ester, propylene glycol dioleate, propylene glycol laurate, glyceryl linoleate, glyceryl diisopalmitate, glyceryl sesquioleate, glyceryl monooleate, glyceryl trimyristate, isopropyl lanolate, hexyldecyl dimethyl octanoate, aceto glyceryl, glyceryl triisooctanoate, glyceryl di-2-heptylundecanoate, glyceryl monopyroglutamate monooleate, decaglyceryl decaoleate, dipentaerythrite fatty acid ester, glyceryl stearate/malate, polyoxyethylene hydrogenated castor oil, polyoxyethylene alkyl (12-14) ether (7E.O.), polyoxyethylene alkyl (12-14) ether (12E.O.), polyoxyethylene alkyl ether (20E.O.), polyoxyethylene lauryl ether, polyoxyethylene myristyl ether, polyoxyethylene cetyl ether, polyoxyethylene cetostearyl ether, polyoxyethyl stearyl ether, polyoxyethylene oleyl ether, polyoxyethylene oleyl cetyl ether, polyoxyethylene behenyl ether, polyoxyethylene octylphenyl ether, polyoxyethylene nonylphenyl ether, polyoxyethylene alkyl (12,13) ether (10E.O.), polyoxyethylene octyldodecyl ether, polyoxyethylene cetyl stearyl diether, polyoxyethylene methylglucoside sesquistearate, carboxylated polyoxyethylene tridecyl ether, mixture of polyethylene glycol-4 octanoate and poly(oxyethylene)nonylphenyl ether (14E.O.), polyoxyethylene lanoline alcohol, polyoxyethylene (5) lanoline alcohol, polyoxyethylene (10) lanoline alcohol, polyoxyethylene (15) lanoline alcohol, polyoxyethylene (20) lanoline alcohol, polyoxyethylene (25) lanoline alcohol, polyoxyethylene (40) lanoline alcohol, polyoxyethylene(alkylol/lanolin alcohol)ether (16E.O.), polyoxyethylene lanolin alcohol acetate, polyoxyethylene lanolin, polyoxyethylene hydrogenated lanolin, polyoxyethylene sorbitol lanolin (40E.O.), polyoxyethylene polyoxypropylene hydrogenated lanolin, polyoxyethylene polyoxypropylene lanolin, polyoxyethylene polyoxypropylene lanolin oil, polyoxyethylene stearic acid amide, polyoxyethylene phytosterol, polyoxyethylene dinonylphenyl ether, polyethylene glycol monolaurate, polyethylene glycol dilaurate, polyethylene glycol palmitate, polyethylene glycol dipalmiate, polyethylene glycol dipalmiate, polyethylene glycol monooleate, polyethylene glycol 150 dioleate, polyethylene glycol monostearate, polyethylene glycol distearate, polyoxyethylene glyceryl tristearate, polyethylene glycol 300 lanolin fatty acid, polyethylene glycol 600 lanolin fatty acid, polyethylene glycol 1000 lanolin fatty acid, polypropylene glycol (36) monooleate, polyoxyethylene (20E.O.) sorbitan monolaurate, polyoxyethylene (20E.O.) coconut oil fatty acid sorbitan, polyoxyethylene (20E.O.) sorbitan monopalmitate, polyoxyethylene (40E.O.) sorbitan oleate, polyoxyethylene (6E.O.) sorbitan monooleate, polyoxyethylene (20E.O.) sorbitan monooleate, polyoxyethylene Sorbitan monolaurate, polyoxyethylene sorbitan tetraoleate, polyoxyethylene (20E.O.) sorbitan trioleate, polyoxyethylene (6E.O.) sorbitan monostearate, polyoxyethylene (20E.O.) sorbitan monostearate, polyoxyethylene (6E.O.) sorbitan tri stearate, polyoxyethylene sorbitan hexastearate, polyoxyethylene (3E.O.) sorbitan isostearate, polyoxyethylene glyceryl monostearate, sorbeth-6 beeswax, polyoxyethylene polyoxypropylene butyl ether, polyoxyethylene (20) polyoxypropylene (15) butyl ether, polyoxyethylene (35) polyoxypropylene (28) butyl ether, polyoxyethylene (36) polyoxypropylene (36) butyl ether, polyoxyethylene (37) polyoxypropylene (38) butyl ether, polyoxyethylene polyoxypropylene cetyl ether, polyoxyethylene polyoxypropylene decyltetradecyl ether, polyoxyethylene polyoxypropylene oligo succinate (3E.O.) (20P.O.), polyoxyethylene polyoxypropylene hexylene glycol ether (300E.O.) (75P.O.), trimethylolpropane tri(polyoxyethylene isostearate) (3E.O.), glyceryl polyoxyethylene oleate, polyoxyethylene (10) polyoxypropylene glycol (30), polyoxyethylene (16) polyoxypropylene glycol (30), polyoxyethylene (25) polyoxypropylene glycol (30), polyoxyethylene(160)polyoxypropylene glycol (30), polyoxyethylene (30) polyoxypropylene glycol (35), polyoxyethylene (200) polyoxypropylene glycol (40), polyoxyethylene (200) polyoxypropylene glycol (70), polyoxyethylene(20)polyoxypropylene glycol (15) butyl ether, polyoxyethylene (6) polyoxypropylene glycol (30), polyoxyethylene (1) polyoxypropylene (4) cetyl ether, polyoxyethylene (1) polyoxypropylene (8) cetyl ether, polyoxyethylene (20) polyoxypropylene (4) cetyl ether, polyoxyethylene(20)polyoxypropylene (8) cetyl ether, polyoxypropylene (9) diglyceryl ether, polyoxypropylene stearyl ether, tetra(polyoxyethylene polyoxypropylene)ethylenediamine, polyoxyethylene (6E.O.) capric/caprylic glyceride, polyoxyethylene glyceryl laurate, polyoxyethylene glyceryl coconut oil fatty acid, polyoxyethylene diethanolamine lauric acid ester (4E.O.), dioctyldodecyl lauroyl glutamate, dipolyoxyethylene stearyl ether diester lauroyl glutamate, polyoxyethylene octyldodecyl ether diester lauroyl glutamate, polyoxyethylene glyceryl pyroglutamic acid isostearate, PEG-40 hydrogenated castor oil PCA isostearate, capric acid diethanolamine, undecylenic acid monoethanolamide, coconut oil fatty acid monoethanolamide, coconut oil fatty acid diethanolamide (1:2 type), coconut oil fatty acid diethanolamide, lauric acid ethanolamide, lauric acid diethanolamide, lauric acid isopropanolamide, lauramide/myristamide diethanolamide (DEA), lauramide/myristamide triethanolamine (TEA), myristic acid diethanolamide, palmitic acid ethanolamide, stearic acid monoethanolamide, stearic acid diethanolamide, stearic acid diethylaminoethylamide, isostearic acid diethanolamide, oleic acid diethanolamide, linoleic acid diethanolamide, tallowamide monoethanolamide, tallowamide diethanolamide, hydrogenated tallowamide diethanolamide, lanolinamide diethylamide, polyoxyethylene coconut oil fatty acid amide (5E.O.), polyoxyethylene (2) coconut oil fatty acid monoethanolamide, polyoxyethylene (5) coconut oil fatty acid monoethanolamide, polyoxyethylene (10) coconut oil fatty acid monoethanolamide, polyoxyethylene coconut oil fatty acid diethanolamide, polyoxyethylene beef tallow alkyl diethanolamine (2E.O.), polyoxyethylene coconut oil alkyl dimethylamineoxide, coconut oil alkyl dimethylamine oxide liquid, oleyldimethylamine oxide, dihydroxyethyl lauramine oxide, pentaerythrityl rosinate, hexaglyceryl oleate, methyl glucose sesquistearate, polyethylene glycol monooleate, polyethylene glycol alkylate, polyethylene alkyl ether, polyglycol diether, lauroyl diethanolamide, fatty acid isopropanolamide, maltitol hydroxy fatty acid ether, alkylated polysaccharide, alkylglucoside; trehalose mono-fatty acid ethers such as trehalose monomyristate, trehalose monoisostearate, and trehalose monoundecylate; polyoxyethylene methyl glucoside dioleate (120E.O.); sugar esters such as sucrose fatty acid ester, glyceryl cocoate, or a combination thereof.

In some embodiments, the at least one surfactant can be a non-ionic surfactant such as lipophilic glycerin monostearate, self-emulsifying glyceryl monostearate, polyglyceryl monostearate, polyglyceryl alkylate, sorbitanmonooleate, polyethylene glycol monostearate, polyoxyethylene sorbitan monooleate, polyoxyethylated sterol, polyoxyethylated lanolin, polyoxyethylated beeswax, polyoxyethylene hydrogenated castor oil, glycerin fatty acid ester, polyglycerin fatty acid ester, polyoxyethylene sorbitan fatty acid ester, polyoxyethylene sorbit fatty acid ester, polyoxyethylene glycerin fatty acid ester, polyoxyethylene alkyl ether, polyoxyethylene fatty acid ester, polyoxyethylene hydrogenated castor oil, polyoxyethylene castor oil, polyoxyethylene-polyoxypropylene copolymer, polyoxyethylene-polyoxypropylene alkyl ether, polyether-modified silicone lauric acid alkanolamide, alkylamineoxide, hydrogenated soybean phospholipid, caprylyl glucoside, glycerol citric acid fatty acid ester, glyceryl stearate citrate, corn oil PEG-8 esters, cocamide DEA, cocamide MEA, cocamide MIPA, sucrose tetrastearate triacetate, hydrogenated palm kernel glycerides, hydrogenated palm PEG-200 glyceride, hydrogenated palm glycerides, sorbitan sesquiisostearate, polyglyceryl-2 sesquiisostearate, polyglyceryl-2 sesquicaprylate, PEG-20 methyl glucose sesquistearate, or a combination thereof.

In some embodiments, the at least one surfactant can be an anionic surfactant such as sodium stearate, zinc stearate, calcium stearate, magnesium stearate, aluminum stearate, aluminum isostearate, triethanolamine stearate, potassium palmitate, sodium cetyl sulfate, sodium lauryl phosphate, disodium lauryl phosphate, triethanolamine palmitate, sodium polyoxyethylene lauryl phosphate, sodium N-acylglutamate, sodium palmitate, sodium laurate, potassium laurate, zinc laurate, triethanolamine laurate, sodium laureate, sodium lauryl sulfate, potassium lauryl sulfate, ammonium lauryl sulfate, monoethanolamine lauryl sulfate, diethanolamine lauryl sulfate, triethanolamine lauryl sulfate, sodium lauryl diaminoethyl glycine, triethanolamine ether alkyl sulfate, turkey red oil, linear dodecyl benzene sulfonate, polyoxyethylene hydrogenated castor oil maleate, acyl methyl taurate, fatty acid soap, α-acylsulfonate, alkyl sulfonate, alkyl allyl sulfonate, alkyl naphthalene sulfonate, alkyl sulfate, POE alkyl ether sulfate, alkyl amide sulfate, alkyl phosphate, POE alkyl phosphate, alkyl amide phosphate, alkyloyl alkyl taurate, N-acyl amino acid salt, POE alkyl ether carbonate, alkylsulfo succinate, sodium alkyl sulfoacetate, acyl isethionate, acylated hydrogenated collagen peptide salt, perfluoroalkyl phosphate, soap noodle, potash soap, zinc undecylenate, soap noodle containing potassium, potassium coconut oil fatty acid, sulfonated castor oil, sodium myristate, potassium myristate, zinc myristate, magnesium myristate, sodium oleate, potassium oleate, N-acyl-L-glutamic acid triethanolamine, sodium N-acyl-L-glutamate, sodium N-coconut oil fatty acid acyl-L-glutamate, sodium N-coconut oil fatty acid/hydrogenated tallowate acyl-L-glutamate, N-coconut oil fatty acid acyl-L-glutamic acid triethanolamine, disodium stearoyl-L-glutamate, sodium N-hydrogenated tallowate acyl-L-glutamate, sodium N-lauroyl-L-glutamate, N-stearoyl-L-glutamate, N-lauroyl-L-lysine, lauroyl-L-glutamic acid triethanolamine, L-arginine ethyl·DL-pyrrolidone carboxylic acid salt, N-coconut oil fatty acid acyl-L-arginine ethyl·DL-pyrrolidone carbonate, cocoyl sarcosinate, sodium cocoyl sarcosinate, N-(cocoyl)sarcosine triethanolamine salt, lauroyl sarcosine, sodium lauroyl sarcosine, lauroyl sarcosine triethanolamine, carboxylated polyoxyethylene tridecyl ether sodium salt (3E.O.), sodium β-laurylaminopropionate, laurylaminopropionate solution, potassium methyl cocoyl taurate, sodium methyl cocoyl taurate, sodium lauroylmethyl taurate, sodium laurylsulfoacetate, sodium cocoyl methyl alanine solution, cocoyl triethanolamine solution, sodium lauroylmethyl-β-alanine solution, disodium lauryl-N-carboxymethoxyethyl-N-carboxymethyl-imidaz olinium dodecanoyl sarcosine, sodium coco alkyl-N-carboxyethyl-N-hydroxyimidazolium betaine, disodium coco alkyl-N-carboxyethoxyethyl-N-carboxyethyl-imidazolinium hydroxide, disodium coco alkyl-N-carboxymethoxyethyl-N-carboxymethyl-imidazo linium hydroxide, disodium coco alkyl-N-carboxymethoxyethyl-N-carboxymethyl-imidazo linium lauroyl sulfate, sodium myristoylmethyl-β-alanine solution, sodium methyl stearoyl taurate, sodium tetradecene sulfonate, sodium dodecylbenzene sulfonate, triethanolamine dodecylbenzene sulfonate, sodium alkanesulfonate, ammonium alkyl sulfate, sodium octylphenoxy diethoxyethyl sulfonate, triethanolamine/magnesium coco alkyl sulfate, sodium myristyl sulfate, sodium cetostearyl sulfate, sodium oleyl sulfate, triethanol amine oleyl sulfate, sodium polyoxyethylene lauryl ether sulfate, ammonium polyoxyethylene lauryl ether sulfate (2E.O.), triethanolamine polyoxyethylene lauryl ether sulfate, ammonium polyoxyethylene alkyl ether sulfate (3E.O.) solution, diethanolamine polyoxyethylene alkyl ether sulfate (3E.O.) solution, triethanolamine polyoxyethylene alkyl ether sulfate (3E.O.) solution, sodium polyoxyethylene alkyl ether sulfate (3E.O.) solution, sodium cocomonoglyceride sulfate, polyoxyethylene (5) coconut fatty acid monoethanolamide phosphate; polyoxyethylenealkyl (12-14) ether phosphate (2E.O.), (8E.O.), (10E.O.); polyoxyethylene laurylether phosphate, sodium polyoxyethylene laurylether phosphate, polyoxyethylene cetylether phosphate, sodium polyoxyethylene cetylether phosphate, polyoxyethylene stearylether, polyoxyethylene cholestearyl ether, polyoxyethylene stearylether phosphate, polyoxyethylene oleylether phosphate, sodium polyoxyethylene oleylether phosphate, diethanolamine polyoxyethylene oleylether phosphate, polyoxyethylene alkylphenylether phosphate, triethanolamine polyoxyethylene alkylphenylether phosphate, polyoxyethylene-polyoxypropylene cetylether phosphate, polyoxypropylene glyceryl ether phosphate, polyoxypropylene butyl ether phosphate, disodium laureth sulfosuccinate, sodium cocoyl ethyl ester sulfonate, dioctyl sodium sulfosuccinate, sodium octylphenoxy diethoxyethyl sulfonate, disodium lauryl sulfosuccinate, disodium oleic acid amidosulfosuccinate, C12-15 pareth-3 phosphate, sodium polyoxyethylene alkyl (C12-14) sulfosuccinate (7E.O.), disodium lauramido PEG-5 sulfosuccinate (5E.O.), sodium isostearoyl lactylate, sodium undecylenoyl hydrolyzed collagen, potassium undecylenoyl hydrolyzed collagen, sodium 2-undecyl-1-hydroxyethyl imidazolinium betaine, sodium lauroyl hydrolyzed collagen, sodium cocoyl hydrolyzed collagen, potassium cocoyl hydrolyzed collagen, potassium cocoyl hydrolyzed collagen solution, TEA-cocoyl hydrolyzed collagen, isostearoyl hydrolyzed collagen, isostearoyl hydrolyzed silk, AMPD-isostearoyl hydrolyzed collagen, ethylenediamine-N,N,N′,N′-tetrakis (2-hydroxylpropyl)dioleate, dodecanoylsarcosine, sodium lauriminodipropionate (30%), oleoyl sarcosine, sodium myristoyl f3-alanine solution, sodium undecylhydroxyethylimidazolinium betaine, ammonium C12,13,16 alkyl sulfate, triethanolamine alkyl (11,13,15) sulfate (1), triethanolamine alkyl (11,13,15) sulfate (2), triethanolamin alkyl (12-15) sulfate, triethanolamin alkyl (12-14) sulfate, sodium alkyl (12, 13) sulfate, triethanolamin polyoxyethylene alkyl ether sulfate (3E.O.) solution, sodium C11-15 pareth-3 sulfate, sodium C12-13 pareth-3 sulfate, sodium C12-15 pareth-3 sulfate, TEA C12-13 pareth-3 sulfate, TEA/Na C12-13 pareth-3 sulfate, sodium PEG-3 cocamide sulfate, sodium PEG-5 cocamide phosphate, disodium PEG-5 laurylcitrate sulfosuccinate, PPG-S-ceteth-10, PEG-25 butyl phosphate, sodium C14-18 alkyl sulfonate, C20-22 alkyl phosphate, isolaureth-4 phosphate, undecylenoyl glycine, potassium olive fatty acid, oleyl sarcosine, sodium oleyl methyl taurate, oleth-3 phosphate, oleth-4 phosphate, oleth-5 phosphate, oleth-10 phosphate, oleth-20 phosphate, sodium oleth-7 phosphate, sodium oleth-8 phosphate, sodium C14-16 olefin sulfonate, capryloyl glycine; cocoyl glutamates such as sodium cocoyl amino acids, cocoyl alanine triethanolamine, sodium cocoyl isethionate, ammonium cocoyl isethionate, potassium cocoyl glycine, sodium cocoyl glycine, triethanolamine cocoyl glutamate, cocoyl glutamate, disodium cocoyl glutamate, and potassium cocoyl glutamate; cocoyl sarcosine, sodium cocoyl sarcosinate, triethanolamine cocoyl sarcosinate, sodium cocoyl taurate, cocoyl methyl f3-alanine, sodium cocoyl methyl f3-alanine, methyl cocoyl taurate, potassium methyl cocoyl taurate, magnesium methyl cocoyl taurate, sodium methyl cocoyl taurate, sodium cocoyl apple amino acids, sodium coceth sulfate, di-C11-15 pareth-2 phosphate, di-C12-15 pareth-4 phosphate, di-C12-15 pareth-8 phosphate, di-C12-15 pareth-10 phosphate, dioleyl phosphate, sodium dioleth-8 phosphate, sodium dioleth-8 phosphate, sodium dicocoylethylenediamine PEG-15 sulfate, sodium dilaureth-10 phosphate, steareth-2 phosphate, steareth-3 phosphate, calcium stearoyl lactylate, sodium stearoyl lactylate, disodium undecylenamido MEA-sulfosuccinate, disodium lauramido MEA-sulfosuccinate, di sodiumlaureth sulfosuccinate, DEA-cetyl phosphate, potassium cetyl phosphate, sodium cetyl stearyl sulfate, ceteth-10 phosphate, ceteth-20 phosphate, triceteareth-4 phosphate, trideceth-4 carboxylic acid, trideceth-8 carboxylic acid, sodium trideceth-4 carboxylic acid, sodium trideceth-7 carboxylic acid, sodium trideceth-3 carboxylic acid, potassium trideceth-7 carboxylic acid, trilaureth-4 phosphate, oleyl lactate, sodium palmoyl glutamate, magnesium palmitoyl glutamate, sodium palmitoyl sarcosinate, palmitoyl proline, sodium palmitoyl proline, sodium methyl palmitoyl taurate, potassium myristoyl glutamate, sodium myristoyl glutamate, sodium myristoyl sarcosinate, sodium methyl myristoyl taurate, potassium cocoate, triethanolamine cocoate, arginine cocoate, disodium lauryliminodiacetate, sodium lauryl glycol carboxylate, laureth-5 carboxylic acid, laureth-6 carboxylic acid, laureth-11 carboxylic acid, sodiumlaureth-5 carboxylate, sodiumlaureth-6 carboxylate, sodiumlaureth-11 carboxylate, laureth-5 acetate, laureth-6 acetate, potassiumlaureth-4,5 acetate, sodiumlaureth-3 acetate, sodiumlaureth-4 acetate, sodiumlaureth-5 acetate, sodiumlaureth-6 acetate, sodiumlaureth-11 acetate, MIPA-laureth sulfate, sodium laureth sulfate, triethanolamine laureth sulfate, ammoniumlaureth-2 sulfate, ammoniumlaureth-3 sulfate, laureth-1 phosphate, laureth-2 phosphate, laureth-4 phosphate, sodium lauroyl aspartate, sodium lauryl oat amino acids, sodium lauroyl lactate, N-lauroyl-N-methyl-β-alanine potassium salt, TEA-lauroyl methylaminopropionate, dicetyl phosphate, cetyl phosphate, or a combination thereof.

In some embodiments, the at least one surfactant can be a cationic surfactant such as lauryl trimethyl ammonium chloride, dicocodimonium chloride, myristyl dimethyl benzyl ammonium chloride, cetyltrimethylammonium chloride, laurylpyridinium chloride, cetylpyridinium chloride, benzethonium chloride, stearyl trimonium chloride, benzalkonium chloride, benzalkonium chloride solution, lauramine oxide, alkyltrimethylammonium chloride, stearyl trimethyl ammonium chloride, stearyl dimethyl benzyl ammonium chloride, dimethyl stearyl ammonium chloride treated hectorite, benzyl dimethyl stearyl ammonium chloride treated hectorite, distearyldimonium chloride, benzyl dimethyl stearyl ammonium chloride treated hectorite, distearyldimonium chloride, distearyldimonium chloride treated bentonite, dialkyldimethyl ammonium chloride, tri(polyoxyethylene)stearyl ammonium chloride (5E.O.), di(polyoxyethylene) oleyl methyl ammonium chloride (2E.O.), N-(stearoyl colamino formyl methyl) pyridinium chloride, polyoxypropylene methyl diethyl ammonium chloride, alkyldiaminoethylglycine hydrochloride solution, alkylisoquinolinium bromide solution, laurtrimonium bromide, ammonium cetyl trimethyl bromide, stearyl trimethyl ammonium bromide, cetyltrimethylammonium saccharin, stearyltrimethylammonium saccharin, polyethyleneglycol·epichlorohydrin·coconut alkylamine·dipropylenetriamine condensate, polyethyleneglycol·epichlorohydrin·tallow alkyl aminedipropylenetriamine condensate, ethyl sulfate lanolin fatty acid aminopropyl ethyl dimethyl ammonium (1), (2); behenyl trimethyl ammonium bromide, behenic acid amide propyl dimethyl hydroxypropyl ammonium chloride, stearic acid diethylaminoethylamide, stearic acid diethylaminoethyl amide, stearic acid dimethylaminopropylamide, lanolin derivative quaternary ammonium salt, PEG-5 oleamine, PEG-2 oleammonium chloride, PEG-2 cocamine, PEG-3 cocamine, PEG-5 cocamine, PEG-10 cocamine, PEG-15 cocamine, PEG-2 dimeadowfoamamidoethylmonium methosulfate, hydroxypropyl arginine lauryl/myristyl ether HCl, ceteartrimonium chloride, C10-40 isoalkylamidopropylethyldimonium ethosulfate, isostearyl ethylimidazolium ethosulfate, caesalpinia spinosa hydroxypropyltrimonium chloride, quaternium-33, quaternium-91, cocamidopropyl PG-dimonium chloride phosphate, cocamidopropyl betainamide MEA chloride, PCA ethyl cocoyl arginate, cocotrimonium methosulfate, trigonella foenum-graecum hydroxypropyltrimonium chloride, myrist/palmitamidobutyl guanidine acetate, di-C12-18 alkyl dimonium chloride, dicocoylethyl hydroxyethylmonium methosulfate, dicocodimonium chloride, distearyldimonium chloride, distearoylethyl hydroxyethylmonium methosulfate, dicetyldimonium chloride, dipalmitoylethyl hydroxyethylmonium methosulfate, dihydroxypropyl PEG-5 linoleammonium chloride, dimethyl PABA amidopropyl laurdimonium tosylate, dimethyl stearamine, stearamidoethyl diethylamine, stearamidopropyl diethylamine, stearalkonium chloride, steartrimonium saccharinate, steartrimonium chloride, steartrimonium bromide, stearoxypropyltrimonium chloride, cetalkonium chloride, cetylpyridinium chloride, cetrimonium chloride, cetrimonium bromide, cetrimonium methosulfate, soytrimonium chloride, palmitamidopropyltrimonium chloride, panthenyl hydroxypropyl steardimonium chloride, hydroxyethyl cetyldimonium phosphate, hydroxypropyltrimonium honey, hydroxypropyl bis-hydroxyethyldimonium chloride, behenamidopropyl dimethylamine, behenamidopropyl dimethylamine, behentrimonium chloride, behentrimonium methosulfate, poloxamine 701, poloxamine 702, poloxamine 704, bishydroxyethyl biscetyl malonamide, sodium palm oil alkyl PG dimonium chloride phosphate, laurtrimonium chloride, laurylpyridinium chloride, linoleamidopropyl PG-dimonium chloride phosphate, or a combination thereof.

In some embodiments, the at least one surfactant can be an ampholytic surfactant such as carboxy betaine-, amide betaine-, sulfo betaine-, hydroxy sulfo betaine-, amido sulfo betaine-, phospho betaine-, aminocarboxylate-, imidazoline derivative-, and amideamine-types including lauryl dimethyl amino acetate betaine, 2-alkyl-N-carboxymethyl-N-hydroxyethylimidazolinium betaine, undecyl-N-hydroxyethyl-N-carboxymethyl-imidazolinium betaine, palm oil alkyl betaine, cocamidopropyl betaine, stearyl dihydroxyethyl betaine, stearyl betaine dimethylamino acetate, bis(stearyl-N-hydroxyethylimidazoline)chloroacetic acid complex, cocoyl hydrolyzed collagen, oleoyl hydrolyzed collagen, hexadecyl hydrolyzed collagen, lauramidopropyl betaine/water, myristoyl hydrolyzed collagen, alkyl aminoethyl glycine chloride solution, and lecithin; and others including PEG-3 lauramine oxide, oleamine oxide, oleyl betaine, capryl/capramidopropyl betaine, cocamine oxide, sodium cocoamphoacetate, sodium cocoamphodiacetate, disodium cocoamphodiacetate, sodium cocoamphopropionate, dihydroxyethyl lauramine oxide, stearamine oxide, stearyl betaine, sodium palm kernelamidoethyl hydroxyethyl amino propionate, palm kernelamidopropyl betaine, C12-14 hydroxyalkyl hydroxyethyl β-alanine, hepta hydroxyethyl carboxylate methylimidazolinium chloride/hepta decyl bis-hydroxyethyl imidazolinium, myristamidopropyl betaine, myristamine oxide, myristyl betaine, lauramidopropylamine oxide, lauramidopropyl hydroxysultaine, lauramidopropyl betaine, sodium lauraminopropionate, lauramine oxide, sodium lauriminodipropionate, lauryl hydroxysultaine, lauryl betaine, sodium lauroamphoacetate, and lauroyl lysine; escin, sodium surfactin, saponin, hydroxylated lecithin, hydrogenated lysolecithin, hydrogenated lecithin, cephalin, phosphatidyl serine, lysolecithin, phospholipid, lecithin, or a combination thereof.

In some embodiments, the at least one surfactant can be sodium laurylglucosides hydroxypropylsulfonate, cocamidopropyl hydroxysultaine, sodium cocoyl glutamate, or combinations thereof.

In some embodiments, the composition can further comprise at least one antimicrobial. Exemplary antimicrobials include phenoxyethanol, methylparaben, ethylparaben, propylparaben, butylparaben, or combinations thereof.

In some embodiments, the composition can further comprise at least one anti-inflammatory. In some embodiments, the anti-inflammatory can be botanically-derived compounds such as allantoin, witch hazel, aloe vera, chamomile, thyme extract, Echinacea, purslane extract, or combinations thereof.

In some embodiments, the composition can further comprise at least one astringent. In some embodiments, the at least one astringent can be citric acid, tannic acid, acetoxy propionic acid, isovaleric acid, aminovaleric acid, ethoxy acetic acid, ethoxypropionate, isoaminovaleric acid, epoxyoleic acid, elaidic acid, aminobutyric acid, erucic acid, oxaloacetic acid, formic acid, icosanoic acid, glucuronic acid, iduronic acid, crotonic acid, chloroisocrotonic acid, isocrotonic acid, acetic acid, dihydrolipoic acid, acidum tartaricum, ethyl crotonic acid, diphenylacetic acid, dimethoxyphthalic acid, ethyl hydroxybutyric acid, succinamic acid, stearic acid, stealoric acid, sorbic acid, tiglic acid, thyroxin, decanoic acid, tropic acid, lactic acid, hydroxyisobutyric acid, hydroxyvaleric acid, pyruvic acid, butyl acetate, butylhydroperoxide, brassidic acid, propiolic acid, propionic acid, bromo isovaleric acid, bromoisobutyric acid, bromopropionic acid, hexanoic acid, hexenoic acid, petroselinic acid, heptadecanoic acid, heptanoic acid, maleanilic acid, maleamic acid, mycolic acid, myristic acid, methacrylic acid, methylvaleric acid, methylthioacetic acid, methyl butyrate, mevalonic acid, melissic acid, mercaptoacetic acid, iodoacetic acid, lauric acid, ricinelaidic acid, ricinoleic acid, linoleic acid, linolenic acid, aconitic acid, oxalosuccinic acid, oxoglutaric acid, adipic acid, calcein, carboxyphenylacetic acid, acetoxysuccinic acid, carbocinchomeronic acid, camphoronic acid, isocamphoronic acid, glutaconic acid, glutaric acid, isocitric acid, crocetin, succinic acid, phthalic acid, fumaric acid, isocinchomeronic acid, diethylenetriamine pentaacetate, isophthalic acid, citramalic acid, dinicotinic acid, itaconic acid, dibromosuccinic acid, dichlorosuccinic acid, ethylmalonic acid, dimethylsuccinic acid, oxalic acid, cinchomeronic acid, hemimellitic acid, pelargonic acid, berberonic acid, benzenehexacarboxylic acid, benzenepentacarboxylic acid, maleic acid, malonic acid, mesaconic acid, mesotartaric acid, mesoxalic acid, methylisophthalic acid, methylsuccinic acid, methylmalonic acid, mercaptosuccinic acid, mellophanic acid, peroxyphthalic acid, malic acid, lutidinic acid, leukotriene bromo anthranilic acid, aminocinnamic acid, isopropyl cinnamic acid, oxanilic acid, arecaidine, caboxylic cinnamic acid, cinnamic acid, gallotannic acid, tranexamic acid, nitrocinnamic acid, hydroxycinnamic acid, methoxycinnamic acid, hydroxyhydrocinnamic acid, phenylcinnamic acid, ellagic acid, orotic aciduria, camphanic acid, quinic acid, hydroxyproline, etiocholanic acid, chaulmoogric acid, carnosine, carbazole acetic acid, quinolinecarboxylic acid, quinolinic acid, quinoline dicarboxylic acid, coumaric acid, chlorophenoxyacetic acid, chenodeoxycholic acid, cholanic acid, cholic acid, santoninic acid, dihydro ortho acid, succinuric acid, thiophencarboxylic acid, tetrahydrofolic acid, dehydrocholic acid, terpenylic acid, hydroxypyrone carboxylic acid, hydroxypyrone dicarboxylic acid, hydroxyphenylacetic acid, hydroxyphenylpropionic acid, phenyldicarboxylic acid, homogentisic acid, mandelic acid, muricholic acid, methyldopa, methylphenylacetic acid, methylfurancarboxylic acid, methyl red, mefenamic acid, lysergic acid, lithocholic acid, lipoic acid, reserpinic acid, retinoic acid, levopimaric acid, glycolic acid, salicylic acid, paraphenol zinc phenolsulfonate; extracts of plants such as Althea officinalis, Arnica montana, Polygonum bistorta, Iris spp., Foeniculum vulgare, Lagerstroemia speciosa, Citrus sinensis, Rubus L., Psidium guajava L., Ribes nigrum, Geranium thunbergii, Crataegus cuneata, Filipendula multijuga, Paeonia lactiflora, Betula platyphylla var. japonica, Lonicera japonica, Equisetum arvense, Hedera helix, Thymus vulgaris, Camellia sinensis, Petroselinum crispum, Hamamelis virginiana, Vitis spp., Centaurea cyanus, Citrus limon, Astragalus sinicus, Sanguisorba officinalis, Cinchona succirubra, Salvia L., Tilia miqueliana, Panax ginseng [Asian ginseng], Juniperus communis, Rosmarinus officinalis L., Hypericum erectum, Ginkgo biloba, Melissa officinalis, Ononis spinosa Linn, Aesculus hippocastanum, Swertia japonica, Allium sativum, Matricaria recutita, Thymus vulgaris, Mentha arvensis var. piperascens, Urtica thunbergiana, Capsicum annuum L., Zingiber officinale, Humulus lupulus, Aesculus hippocastanum, Lavandula angustifolia, Daucus carota subsp. sativus, Brassica juncea, Cinnamomum cassia B1., Pinus, Cnidium officinale Makino, Sambucus sieboldiana, Ostericum sieboldii, Scopolia japonica, Paeonia suffruticosa, Myrica rubra, Houttuynia cordata, Nuphar japonicum, Diospyros spp., Calendula officinalis, Papaver rhoeas L., Gentiana scabra var. buergeri, Glehnia littoralis, Citrus aurantium, Citrus junos, Acorus calamus, Citrus natsudaidai, Mehlotus officinalis, Zanthoxylum piperitum, Eucalyptus globulus, Artemisia indica var. maximowiczii, Rabdosia japonica, Oryza sativa, Sophora flavescens, Zingiber officinale, Eugenia caryophyllata, leaf of Juglans, Scutellaria baicalensis, Salvia officinalis, Rosmarinus officinalis L., Polygonum multiflorum Thunb., Coptis japonica (Thunb.) Makino, Phellodendron amurense Rupr., Scutellaria baicalensis Georgi, Houttuynia cordata, Aurantii nobilis pericarpium, Daucus carota subsp. sativus, Juncus effusus L. var decipiens Buch, and Alisma rhizome; plant extracts of tannin and tar of Betula platyphylla; propolis, royal jelly, yeast extract, tuberosa polysaccharide solution, oolong tea, alum, zinc chloride, zinc sulfate, aluminum sulfate, aluminum chloride, aluminum chloride hydroxide allantoin, dihydroxyaluminum allantoin, aluminum hydroxychloride, zinc oxide, zinc sulfide, zinc sulfocarbolate, sodium sulfocarbolate, organic composites of aluminum or zirconium, or a combination thereof.

In some embodiments, the skincare composition can further comprises at least one buffer. In some embodiments, the at least one buffer can be citric acid, sodium citrate, lactic acid, sodium actate, glycollic acid, succinic acid, acetic acid, sodium acetate, potassium acetate, malic acid, tartaric acid, fumaric acid, phosphoric acid, hydrochloric acid, sulfuric acid, boric acid, borax, nitrilotriethanol, monoethanolamine, diethanolamine, triethanolamine, isopropanolamine, triisopropanolamine, 2-amino-2-methyl-1,3-propanediol, 2-amino-2-hydroxymethyl-1,3-propanediol, arginine, ammonium hydroxide, sodium hydroxide, potassium hydroxide, calcium hydroxide, magnesium hydroxide, potassium hydrogen phosphate in ammonia solution, guanidine carbonate, ammonium bicarbonate, sodium carbonate, potassium carbonate, potassium bicarbonate, sodium bicarbonate, magnesium oxide, calcium oxide, ammonium phosphate, disodium hydrogen phosphate, sodium dihydrogen phosphate, potassium phosphate, magnesium phosphate, calcium phosphate, sodium borate, sodium metaborate, potassium citrate, or a combination thereof.

In some embodiments, the skincare composition can further comprise at least one ultraviolet absorbing agent. In some embodiments, the at least one ultraviolet absorbing agent can be p-aminobenzoic acid ultraviolet absorbing agents such as p-aminobenzoic acid, p-aminobenzoic acid monoglyceryl ester, N,N-dipropoxyparaaminobenzoic acid ethyl ester, N,N-diethoxyparaaminobenzoic acid ethyl ester, N,N-dimethylparaaminob enzoic acid ethyl ester, and N,N-dimethylparaaminobenzoic acid butyl ester; anthranilic acid ultraviolet absorbing agents such as homomenthyl-N-acetylanthranilate; benzophenone derivatives such as cinnamic acid, oxbenzone-3, oxbenzone-4, oxbenzone-5, and oxbenzone-9; saccharide ultraviolet absorbing agents wherein saccharides or sugar alcohols bind to 1,3-propanediol derivatives; salicylic acid ultraviolet absorbing agents such as salicylic acid, sodium salt thereof, amyl salicylate, menthyl salicylate, homomenthyl salicylate, octyl salicylate, phenyl salicylate, benzyl salicylate, p-isopropanolphenyl salicylate, and dipropylene glycol salicylate; cinnamic acid ultraviolet absorbing agents such as octyl cinnamate, ethyl-4-isopropylcinnamate, methyl-2,5-diisopropylcinnamate, ethyl-2,4-diisopropylcinnamate, methyl-2,4-dii sopropylcinnamate, propyl-p-methoxycinnamate, isopropyl-p-methoxycinnamate, isoamyl-p-methoxycinnamate, 2-ethylhexyl p-methoxycinnamate, 2-ethoxyethyl p-methoxycinnamate (cinoxate), cyclohexyl p-methoxycinnamate, ethyl-α-cyano-β-phenylcinnamate, 2-ethylhexyl-α-cyano-β-phenylcinnamate (octocrylene), glycerylmono-2-ethylhexanoyl-diparamethoxycinnamate, and ferulic acid and its derivatives; benzophenone ultraviolet absorbing agents such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone; dibenzoylmethane derivatives such as 3-(4′-methylbenzylidene)-d,l-camphor, 3-benzylidene-d,l-camphor, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methylphenyl)benzotriazole, dibenzalazine, dianysoylmethane, 5-(3,3-dimethyl-2-norbornyliden)-3-pentane-2-on, 4-methoxy-4′-t-butylbenzoylmethane, and 4-t-butylmethoxydibenzoylmethane; urocanic acid ultraviolet absorbing agents such as octyltriazone, urocanic acid, and ethyl urocanate; hydantoin derivatives such as 2-(2′-hydroxy-5′-methylphenyl)benzotriazole, 1-(3,4-dimethoxyphenyl)-4,4-dimethyl-1,3-pentanedione, octyl dimethoxybenzylidene dioxoimidazolidine propionate, and 2-ethylhexyl dimethoxybenzylidene dioxoimidazolidine propionate; and others such as phenylbenzimidazole sulfonic acid, terephthalylidene dicamphor sulfonic acid, drometrizole trisiloxane, methyl anthranilate, rutin and derivatives thereof, oryzanol and derivatives thereof, octyldimethyl p-aminobenzoate, titanium oxide, zinc oxide, kaolin, talc, ethylhexyltriazone, octocrylene, octyl dimethyl PABA, homosalate, methylene bisbenzotriazoryl tetramethylbutyl phenol, ethylhexyl methoxycinnamate, octyl methoxycinnamate, or a combination thereof.

In some embodiments, the skincare composition can further comprise at least one thickener. In some embodiments, the at leat one thickener can be guar gum, locust bean gum, quince seed, carrageenan, galactan, gum arabic, tara gum, tamarind, furcelleran, gum karaya, Abelmoschus manihot, cara gum, tragacanth gum, pectin, pectinic acid, and salts thereof such as of sodium salts; alginic acid and salts thereof such as of sodium salts; starches such as mannan, rice, corn, potato, and wheat; dextrin, xanthan gum, dextran, succinoglucan, curdlan, hyaluronic acid, and salts thereof; xanthan gum, pullulan, gellan gum, chitin, chitosan, agar, brown algae extract, chondroitin sulfate, casein, collagen, gelatin, albumin, methyl cellulose, ethyl cellulose, hydroxypropyl cellulose, hydroxyethyl cellulose, carboxymethyl cellulose, and their salts such as of sodium salts; cellulose and derivatives thereof such as methyl hydroxypropyl cellulose, sodium cellulose sulfate, dialkyl dimethyl ammonium cellulose sulfate, crystalline cellulose, and cellulose powder; starch high molecules such as soluble starch, carboxymethyl starch, methyl hydroxypropyl starch, and methyl starch; starch derivatives such as starch hydroxypropyltimonium chloride, and aluminum corn starch octenylsuccinate; algin acid derivatives such as propylene glycol alginate; amphoteric methacrylate copolymers such as polyvinyl pyrrolidone (PVP), polyvinyl alcohol (PVA), vinyl pyrrolidone-vinyl alcohol copolymer, vinyl alcohol-vinyl acetate copolymer, polyvinyl methyl ether, polyethylene glycol, polypropylene glycol, polyoxyethylene-polyoxypropylene copolymer, (methacryloyloxyethyl carboxy betaine/alkyl methacrylate)copolymer, acrylates/stearyl acrylate/ethylamine oxide methacrylate copolymer; dimeticone/vinyl dimethicone crosspolymer, (alkyl acrylate/diacetone acrylamide)copolymer, (alkyl acrylate/diacetone acrylamide)copolymer AMP, partially saponified polyvinyl acetate, maleic acid copolymer, polyvinylpyrrolidone, vinylpyrrolidone-methacrylate dialkylaminoalkyl copolymer; alkanolamine acrylic resin; polyester; water-dispersible polyester; polyacrylamide; polyacrylic ester copolymers such as ethyl polycrylate; carboxy vinyl polymer; polyacrylic acid including salts thereof such as of sodium salts; acrylic acid/methacrylic acid copolymer; acrylic acid/methacrylic acid alkyl copolymer; cationized celluloses such as polyquaternium-10; diallyldimethylammonium chloride/acrylamide copolymers such as polyquaternium-7; acrylic acid/diallyldimethylammonium chloride copolymers such as polyquaternium-22; acrylic acid/diallyldimethylammonium chloride/acrylamide copolymers such as polyquaternium-39; acrylic acid/cationized methacrylate copolymer; acrylic acid/cationized methacrylamide copolymer; acrylic acid/methylacrylate/methacrylamidepropyltrimethyl-a mmonium chloride copolymer such as polyquaternium-47; chlorinated methacrylic acid choline ester copolymer; cationated polysaccharides such as cationated oligosaccharides, cationated dextrans, and guar hydroxypropyltrimonium chloride; polyethylenimine; cationic polymer; 2-methacryloyloxyethyl phosphorylcholine copolymers such as polyquaternium-51; high molecular emulsions such as acrylic resin emulsion, polyacrylic acid ethyl emulsion, polyacrylalkylester emulsion, polyvinylacetate resin emulsion, natural rubber latex, and synthetic latex; cellulose nitrate, polyurethanes, and copolymers thereof; silicon copolymers such as silicons, acryl/silicon graft copolymers; fluorine high molecules; 12-hydroxystearic acid and salts thereof; dextrin fatty esters such as dextrin palmitate and dextrin myristate; hydroxystearic acid cholesteryl; silicic anhydride; fumed silica (ultrafine silicic anhydride); magnesium aluminum silicate; sodium magnesium silicate; metal soap; dialkyl phosphoric acid metal salt; bentonite; hectorite; organophilic clay minerals; sucrose fatty acid ester; fructooligosaccharide fatty acid ester; seed extract of Cydonia oblonga; and natural, semisynthetic, and synthetic high-molecular substances such as hydroxyethyl guar gum, carboxymethyl guar gum, starch, carboxymethyl chitin, or a combination thereof.

In some embodiments, the composition can further comprise at least one amino acid. In some embodiments, the at least one amino acid can be aline, leucine, isoleucine, threonine, methionine, phenylalanine, tryptophan, lysine, glycine, alanine, asparagine, glutamine, serine, cysteine, cystine, tyrosine, proline, hydroxyproline, asparaginic acid, glutamic acid, hydroxylysine, arginine, ornithine, histidine, taurine, carnitine, and their sulfates, phosphates, nitrates, citrates, pyrrolidone carboxylates, and derivatives thereof, or a combination thereof.

In some embodiments, the composition can further comprise at least one percutaneous-absorption accelerator. In some embodiments, the at least one percutaneous-absorption accelerator can be urea; α-hydroxylic acids such as lactic acid, fruit acid, and glycolic acid; β-hydroxylic acids such as salicylic acid; and others such as oleic acid, undecanoic acid, octanol, nonanol, menthol, thymol, limonene, dimethylsulfoxide, dodecyl methyl sulfoxide, dimethylacetamide, N,N-dimethylformamide, sodium lauryl sulfate, N,N-bis(2-hydroxyethyl)oleylamine, polyoxyethylene (20)sorbitan monooleate, dodecyldimethylammonium propanesulfonic acid, propylene glycol, polyethylene glycol, N,N-dimethyl-m-toluamide, N,N-diethyl-m-toluamide, laurocapram, 1-dodecyl azacyclohepthane-2-on, isopropyl myristate, Isopropyl palmitate, N-mono- or di-substituted p-menthane-3-carboxyamide, 2-(2-methoxy-1-methylethyl)-5-methylcyclohexanol, azacycloalkane derivatives, cyclodextrins, or a combination thereof.

In some embodiments, the skincare composition can further comprise at least one foaming agent. In some embodiments, the at least one foaming agent can be azodicarbonamide, barium azodicarboxylate, azobisisobutyronitrile, azodicarbonic acid amide, N,N′-dinitrosopentamethylenetetramine, N,N′-dimethyl-N,N′-dinitrosoterephtalamide, trinitrotrimethyltriamine, 4,4′-oxybis(benzenesulfonyl hydrazide), p-toluenesulfonylhydrazide, diphenyl sulfone-3,3′-sulfonyl hydrazide, arylbis(sulfonyl hydrozide), p-toluylenesulfonyl semicarbazide, and 4,4′-oxybis(benzene sulfonyl semicarbazide); alkane fluorides such as trichlorofluoromethane and dichloromonofluoromethane; 5-morphoryl-1,2,3,4-thiatriazole, ammonium carbonate, ammonium hydrogen carbonate, sodium hydrogen carbonate, ammonium nitrite, sodium borohydride, azides, or a combination thereof.

In some embodiments, the composition can further comprise at least one plasticizer. In some embodiments, the at least one plasticizer can be ethanol, phenoxyethanol, isopropanol, butyl alcohol, benzyl alcohol, lauryl alcohol, myristyl alcohol, cetyl alcohol, cetostearyl alcohol, stearyl alcohol, oleyl alcohol, behenyl alcohol; polyols such as ethylene glycol, diethylene glycol, triethylene glycol, polyethylene glycol, propylene glycol, dipropylene glycol, polypropylene glycol, glycerine, diglycerine, polyglycerine, glyceryl monostearate, isopropylene glycol, 1,2-buthane diol, 1,3-buthane diol, 1, 4-buthane diol, 2,3-buthane diol, 1,2-penthane diol, 1,5-penthane diol, 2,3-penthane diol, 2,4-penthane diol, sorbitol, maltitol, raffinose, hexylene glycol, lauric acid diethanolamide, fatty acid diethanolamide, or a combination thereof.

Soaps

In some embodiments, the composition described herein can be a cleansing composition or soap, including traditional soaps in the form of solid bar and liquid soaps in the form of cleaners, makeup removers, body washes, milks, creams, foams cream gels, or gels that can be packaged in tubes, bottles, pump bottles, aerosol shower foams or foam pump bottles. Soaps can be used in a cosmetic process for cleaning the dirt residues of human keratinous materials in the presence of water, massed to form a foam and the formed foam and the soil residues are removed by rinsing with water, and can be used on any part of the body and face disclosed herein, including, for example, on the skin of the body, face, hands, lips, eyelids, nails, hair, eyelashes and/or eyebrows.

Traditionally, solid soaps include alkali metal fatty acid salts and potassium fatty acid soaps, and liquid soaps include four main families of detergent formulation: (1) those based on lauryl sulfate; those based on alpha-olefin sulfonate, (3) those based on a mixture of synthetic anionic, amphoteric and/or nonionic surfactants; (4) mixed formulations based on soaps and synthetic surfactants. Liquid soaps generally contain a thickening system chosen, for example, from electrolytes such as sodium chloride, potassium chloride or potassium sulphate; alkanolamides such as cocamide DEA or cocamide MEA; esters of polyethylene glycol and monoacid or stearic acid such as polyethylene glycol distearate 6000 or mixtures thereof, and are contained in a cosmetically acceptable aqueous medium. However, both solid and liquid soaps can comprise any suitable additional ingredients such as those listed below herein, in any combination.

In some embodiments, the cleansing composition or soap described herein can further comprise one or more cellulosic compounds, or polysaccharide compound having in its structure chains of glucose residues linked by β-1,4 bonds, one or more fatty acids comprising a linear or branched, saturated or unsaturated alkyl chain having from 6 to 30 carbon atoms or 12 to 22 carbon atoms, one or more fatty acids, including lauric acid, myristic acid, palmitic acid and stearic acid, linolenic acid, and mixtures thereof, and one or more mineral bases, including alkali metal hydroxides (sodium hydroxide and potassium hydroxide), metal hydroxides or ammonia or organic bases such as triethanolamine, monethanolamine, monoisopropanolmaine, N-methylglucamine, lysine and arginine.

In some embodiments, the composition described herein can further comprise one or more anionic surfactants or salts, including alkali metal salts such as sodium salts, ammonium salts, amine salts, aminoalcohol salts or salts. alkaline earth metals, for example, magnesium, of the following types: alkyl sulphates, alkyl ether sulphates, alkyl amido ether sulphates, alkyl aryl polyether sulphates, monoglyceride sulphates; alkylsulfonates, alkylamidesulfonates, alkylarylsulphonates, α-olefin-sulfonates, paraffin-sulfonates; alkyl sulfosuccinates, alkylethersulfosuccinates, alkylamide-sulfosuccinates; alkylsulfoacetates; acylsarcosinates; and the acylglutamates, the alkyl and acyl groups of all these compounds having from 6 to 24 carbon atoms and the aryl group denoting a phenyl or benzyl group, C6-C24 alkyl esters of polyglycoside carboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all these compounds having from 12 to 20 carbon atoms, and/or acyllactylates whose acyl group contains from 8 to 20 carbon atoms and their mixtures. In some embodiments, alkyl-D-galactoside uronic acids, polyoxyalkylenated (C6-C24) ether carboxylic acids, polyoxyalkylenated (C6-C24) aryl (C6-C24) polyoxyalkylenated ether carboxylic acids, polyoxyalkylenated (C6-C24) alkyl amidoether carboxylic acids, in particular those containing from 2 to 50 ethylene oxide groups; and their alkali metal, ammonium, amine, aminoalcohol or alkaline earth metal salts can also be suitable.

Suitable C6-C24 alkyl ether sulphate salts containing from 1 to 30 ethylene oxide groups, include alkali metals or alkaline earth metals, ammonium, amine or amino alcohol salts, sodium salts and oxyethylenated (C12-C14) alkyl ethersulfates having an average number of ethylene oxide groups of between 1 and 4 and including sodium laureth sulfate (CTFA name) such as the commercial product sold under the name TEXAPON AOS 225 UP TEXAPON N702 TEXAPON NSW marketed by COGNIS or EMPICOL ESB3/FL2, EMPICOL ESB3/FL3, EMPICOL ESB70/FL2 sold by the company Huntsman.

Suitable amphoteric surfactants include, but are not limited to, derivatives of secondary or tertiary aliphatic amines, in which the aliphatic group is a linear or branched chain comprising from 8 to 22 carbon atoms. The amphoteric surfactants can contain at least one water-soluble anionic group such as a carboxylate, sulfonate, sulfate, phosphate or phosphonate group, (C8-C20) alkylbetaines, sulphobetaines, (C8-C20) alkylamido (C6-C8) alkylbetaines or (C8-C20) alkylamidoalkyl (C6-C8) sulfobetaines, and mixtures thereof.

Suitable amine derivatives include the products sold under the name MIRANOL®, as described in patents U.S. Pat. Nos. 2,528,378 and 2,781,354 and filed in the CTFA dictionary, 3rd edition, 1982, under the terms Amphocarboxy-glycinate and Amphocarboxypropionate denominations. Additional suitable amine derivatives include those classified in the CTFA dictionary, 5th edition, 1993, under the names cocoamphodiacetate disodium, lauroamphodiacetate disodium, caprylamphodiacetate disodium, capryloamphodiacetate disodium, cocoamphodipropionate disodium, lauroamphodipropionate disodium, caprylamphodipropionate disodium, capryloamphodipropionate disodium, acid lauroamphodipropionic, cocoamphodipropionic acid, and cocoamphodiacetate sold under the trade name MIRANOL® C2M concentrated by Rhodia. Suitable alkyl (C8-C20) betaines include cocamidopropyl betaine and cocobetaine such as the commercial products MIRATAINE BB/FLA from RHODIA or EMPIGEN BB/FL from Huntsman.

In some embodiments, the cleansing compositions or soaps described herein can be sulfate-free, and can include a sulfate-free surfactant system.

In some embodiments, the cleansing composition or soap described herein can further comprise one or more thickeners of the nonionic cellulosic compound type. Suitable cellulosic compounds include, but are not limited to, nonionic cellulose ethers, including methylcelluloses and ethylcelluloses; hydroxyalkylcelluloses such as hydroxymethylcelluloses, hydroxyethylcelluloses and hydroxypropylcelluloses; mixed hydroxyalkyl-alkylcellulose celluloses such as hydroxypropyl-methylcelluloses, hydroxyethyl-methylcelluloses, hydroxyethylethylcelluloses and hydroxybutyl-methylcelluloses and hydroxyalkylcelluloses modified with an alkyl chain. Suitable hydroxypropyl methylcelluloses include the commercial products METHOCEL E, F, J and K sold by Dow Corning and even more particularly METHOCEL E 4MQG or METHOCEL F 4M. Suitable cellulosic ingredients can be in a crystalline form, a microcrystalline form, or a mixture thereof.

In some embodiments, the cleansing composition or soap described herein can further comprise one or more additional thickeners including electrolytes such as sodium chloride, potassium chloride or potassium sulphate; alkanolamides such as cocamide DEA or cocamide MEA; esters of polyethylene glycol and monoacid or stearic acid such as polyethylene glycol distearate 6000 or mixtures thereof, polysaccharide biopolymers such as xanthan gum, guar gum, alginates, synthetic polymers such as polyacrylics such as CARBOPOL 980, CARBOPOL 1382 marketed by NOVEON, acrylate/acrylonitrile copolymers such as HYPAN SS201 marketed by KINGSTON, clays such as smectites, modified or unmodified hectorites such as BENTONE products marketed by Rheox, LAPONITE products marketed by Southern Clay Products, VEEGUM HS product marketed by RT Vanderbilt, and mixtures thereof.

In some embodiments, the cleansing composition or soap described herein can further comprise one or more nonionic surfactants. These are well-known compounds (see regard “Handbook of Surfactants” by MR PORTER, Blackie & Son editions (Glasgow and London), 1991, pp 116-178), and can be chosen from alcohols, alpha-diols, (C1-C20) alkyl phenols or polyethoxylated, polypropoxylated or polyglycerolated fatty acids, having a fatty chain comprising, for example, from 8 to 18 atoms, the number of ethylene oxide groups or propylene oxide may range from 2 to 50 and the number of glycerol groups may range from 2 to 30, copolymers of ethylene oxide and propylene, condensates of ethylene oxide and propylene oxide on fatty alcohols; polyethoxylated fatty amides having from 2 to 30 moles of ethylene oxide, polyglycerolated fatty amides comprising on average 1 to 5 glycerol groups; polyethoxylated fatty amines having 2 to 30 moles of ethylene oxide, ethoxylated sorbitan fatty acid esters having 2 to 30 moles of ethylene oxide; sucrose fatty acid esters, polyethylene glycol fatty acid esters, (C 6-C 24) alkyl polyglycosides, N-alkyl (C6-C24) glucamine derivatives, amine oxides such as that the oxides of alkyl (C10-C14) amines or the oxides of N-acyl (C10-C14)-aminopropylmorpholine, and mixtures thereof.

Additional suitable nonionic surfactants include, but are not limited to, alkyl polyglucosides (APG), maltose esters, polyglycerolated fatty alcohols, glucamine derivatives, for instance 2-ethylhexyloxycarbonyl-N-methylglucamine, and mixtures thereof. Suitable alkylpolyglucosides include those that contain an alkyl group comprising from 6 to 30 carbon atoms, and a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UP® by the company Cognis.

Suitable maltose derivatives include those described in document EP-A-566 438, such as O-octanoyl-6′-D-maltose or O-dodecanoyl-6′-D-maltose described in document FR-2 739 556.

In some embodiments, the cleansing composition or soap can be formulated in a cosmetically acceptable aqueous medium. Suitable cosmetically acceptable aqueous media can include, in addition to water, one or more solvents such as lower alcohols containing from 1 to 6 carbon atoms, such as ethanol; polyols such as glycerine; glycols such as butylene glycol, isoprene glycol, propylene glycol, polyethylene glycols such as PEG-8, sorbitol, sugars such as glucose, fructose, maltose, lactose, sucrose, and mixtures thereof. The amount of solvent(s) in the composition disclosed herein can range from 0.1 to 95% by weight.

In some embodiments, the cleansing composition or soap described herein can further comprise one or more cationic polymers of the polyquaternium type, which can provide softness and lubricity to a foaming composition. Suitable cationic polymers include Polyquaternium 5 such as the product MERQUAT 5 marketed by the company CALGON, Polyquaternium 6 such as the product SALCARE SC 30 marketed by the company CIBA, and the product MERQUAT 100 marketed by the company CALGON, Polyquaternium 7 such as the MERQUAT S, MERQUAT 2200 and MERQUAT 550 products marketed by the company CALGON, and the SALCARE SC 10 product marketed by the company CIBA, Polyquaternium 10 such as the product Polymer JR400 marketed by the company Amerchol, Polyquaternium 11 such as GAFQUAT 755, GAFQUAT 755N and GAFQUAT 734 products marketed by ISP, Polyquaternium 15 such as the product ROHAGIT KF 720 F marketed by the company ROHM, Polyquaternium 16 such as LUVIQUAT FC905, LUVIQUAT FC370, LUVIQUAT HM552 and LUVIQUAT FC550 products marketed by BASF, Polyquaternium 22 such as the product Merquat 280 sold by the company Calgon, Polyquaternium 28 such as the product STYLEZE CC10 marketed by the company ISP, Polyquaternium 39 such as the MERQUAT PLUS 3330 product marketed by Calgon, Polyquaternium 44 such as the product LUVIQUAT CARE sold by the company BASF, Polyquaternium 46 such as the product LUVIQUAT HOLD marketed by the company BASF, Polyquaternium 47 such as the product MERQUAT 2001 marketed by Calgon, and cationic guars such as the product Jaguar marketed by the company Rhodia can also be used as cationic polymer.

In some embodiments, the cleansing composition or soap described herein can further comprise one or more adjuvants or additives used in cosmetic compositions. Suitable adjuvants or additives include but are not limited to: oils, active agents, perfumes, preservatives, sequestering agents, pearlescent or opacifying agents, pigments, pearlescent agents, mineral or organic fillers such as talc, kaolin, silica powders or of polyethylene, soluble dyes, or any combination thereof.

Examples of oils include vegetable oils (jojoba, avocado, sesame, sunflower, corn, soy, safflower, grape seed), mineral oils (vaseline, isoparaffins optionally hydrogenated), synthetic oils (isopropyl myristate, cetearyl octanoate, polyisobutylene, ethyl hexyl palmitate, alkyl benzoates), volatile or non-volatile silicone oils such as polydimethylsiloxanes (PDMS) and cyclodimethylsiloxanes or cyclomethicones, and fluorinated or fluorosilicone oils and mixtures thereof.

Exemplary active agents include sunscreens, desquamating agents, moisturizing agents, depigmenting agents, pro-pigmentants, alpha-hydroxy acids, antibacterial agents, antiradical agents, anti-pollution agents, anti-inflammatories, retinoids, extracts of algae, mushrooms, vegetables, yeasts, bacteria, hydrolysed, partially hydrolyzed or unhydrolyzed proteins, enzymes, hormones, vitamins and their derivatives, flavonoids and isoflavones, and mixtures thereof.

The cleansing composition or soap described herein can have a pH ranging from 6 to 10 depending on the application chosen. The adjustment of the pH to the desired value can be done conventionally by adding a base (organic or inorganic) in the composition, for example ammonia or a primary, secondary or tertiary (poly) amine such as monoethanolamine, diethanolamine, triethanolamine, isopropanolamine or 1,3-propanediamine, or by addition of a mineral or organic acid, such as a carboxylic acid, for example, citric acid. In the context of shower gels, the pH can vary from 8 to 10.

In some embodiments, the cleansing composition described herein can further comprise optional additives such as colorants, fragrances, antibacterials, preservatives, antioxidants, beads (fragrance, exfoliating or moisturizing), mica, glitter, shea butter, shea butter beads, opacifying agents, pearlizing agents and other such ingredients. In some embodiments, the composition has high clarity (about 2 to about 25 NTU's), a targeted viscosity (about 4,000 to about 10,000 centipoise) for ease of dispensing from an orifice in the range of about ¼ to about ⅛, and a yield value (about 3 to about 15 Pascals) that allows the composition to suspend a variety of additives with a uniformity of distribution and enhanced stability (for example, about 8 months at 120° F. (49° C.), and any mixture thereof.

In some embodiments, the cleansing composition or soap can further comprise one or more moisturizers/emollients. Moisturizers can be included in bar or liquid soap compositions to provide conditioning benefits to the skin. The term “moisturizer” describes a material which imparts a smooth and soft feeling to the skin surface.

There are two ways of reducing water loss from the stratum corneum. One is to deposit on the surface of the skin an occlusive layer which reduces the rate of evaporation. The second method is to add nonocclusive hygroscopic substances to the stratum corneum which will retain water, and make this water available to the stratum corneum to alter its physical properties and produce a cosmetically desirable effect. Nonocclusive moisturizers also function by improving the lubricity of the skin.

Both occlusive and nonocclusive moisturizers are contemplated for use in the compositions described herein. Exemplary moisturizers include long chain fatty acids, liquid water-soluble polyols, glycerin, propylene glycol, sorbitol, polyethylene glycol, ethoxylated/propoxylated ethers of methyl glucose (e.g., methyl gluceth-20), ethoxylated/propoxylated ethers of lanoline alcohol (e.g., Solulan-75® available from the Amerchol Co.) coconut and tallow fatty acids, liquid water-soluble polyols (e.g., glycerin, propylene glycol, butylene glycol, hexylene glycol, polypropylene glycol and polyethylene glycol).

Nonocclusive moisturizers can naturally occur in the stratum corneum of the skin, such as sodium pyrrolidone carboxylic acid, lactic acid, urea, L-proline, guanidine and pyrrolidone. Examples of other nonocclusive moisturizers include hexadecyl, myristyl, isodecyl or isopropyl esters of adipic, lactic, oleic, stearic, isostearic, myristic or linoleic acids, as well as many of their corresponding alcohol esters (sodium isostearoyl-2-lactylate, sodium capryl lactylate), hydrolyzed protein and other collagen-derived proteins, aloe vera gel and acetamide MEA (N-acetyl ethanolamine). Other examples of both occlusive and nonocclusive types of moisturizers are disclosed in “Emollients—A Critical Evaluation,” by J. Mausner, Cosmetics & Toiletries, May 1981, incorporated herein by reference.

Exemplary occlusive moisturizers include petrolatum, mineral oil, beeswax, silicones, lanolin and oil-soluble lanolin derivatives, saturated and unsaturated fatty alcohols such as behenyl alcohol, squalene and squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Hair Care

In some embodiments, the composition can be a hair care composition comprising ingredients commonly used for hair care products. These ingredients can include, but are not limited to, cleansing agents, lathering agents, hydration agents, surfactants, detergents, gelling agents, fragrances, botanical extracts, conditioning agents, humectants, silicones or silicone derivatives, thickening agents, sun blocks, vitamins, alcohols, polyols, polyolethers, and other commonly used ingredients in shampoos, conditioners, and styling agents. Hair care products generally include one or more surfactants, one or more viscosity adjusting agents, one or more preservatives, and one or more fragrances, and any of the ingredients listed below or combinations thereof.

Generally, hair can be damaged and embrittled by the action of external atmospheric agents such as light and bad weather, but also by mechanical or chemical treatments, such as brushing, combing, dyeing, bleaching, permanent-waving and/or relaxing, and over time, hair can become dry, coarse, dull, and/or fragile. To overcome these drawbacks, it is common practice to use care compositions that condition the hair appropriately, giving it satisfactory cosmetic properties, especially in terms of smoothness, sheen, softness, suppleness, lightness, a natural feel and good disentangling properties. These hair care compositions may be, for example, shampoos, conditioning shampoos, hair conditioners, masks, sera, gels, hair lotions and creams that can be rinsed-out or leave-in compositions. In various embodiments, these compositions generally comprise a combination of cationic conditioning agents such as cationic surfactants, cationic polymers, silicones and/or fatty substances, such as fatty alcohols, in order to give the hair satisfactory cosmetic properties, in terms of softness, smoothness and suppleness. Exemplary compositions can comprise silicones, which are known to improve the cosmetic properties of hair in terms of smoothness and flexibility (as shown in U.S. Pat. No. 5,374,421, each of which is incorporated herein by reference).

In some embodiments, the hair care composition can further comprise one or more non-amino polyalkylsiloxanes, one or more oxyethylenated polymers in the presence of fatty alcohols, one or more non-amino polyalkylsiloxanes comprising at least one alkyl chain having at least 12 carbon atoms, one or more oxyethylenated polymers, and/or one or more fatty alcohols. Exemplary oxyethylenated polymers can have a weight-average molecular mass greater than or equal to 106. In some embodiments, the oxyethylenated polymers can be chosen from the compounds having the formula H(OCH2CH2)zOH, wherein, z is an integer greater than or equal to 30,000. In certain embodiments, z can range from 30,000 to 120,000, or from 40,000 to 95,000. In some embodiments, the oxyethylenated polymer can be PEG-45M (z=45,000) such as the product sold under the name Polyox WSR N 60 K by the company Amerchol, and PEG-90M (z=90 000), and mixtures thereof.

In some embodiments, the hair care composition can further comprise one or more fatty alcohols. The term “fatty alcohol” means any saturated or unsaturated, linear or branched alcohol comprising at least 8 carbon atoms and which is not oxyalkylenated. Exemplary fatty alcohols are solid at room temperature (25° C.) and at atmospheric pressure (1.013*105 Pa). Exemplary fatty alcohols include cetyl alcohol, stearyl alcohol, oleyl alcohol, behenyl alcohol, linoleyl alcohol, palmitoleyl alcohol, arachidonyl alcohol, erucyl alcohol, cetylstearyl (or cetearyl) alcohol, and mixtures thereof.

In some embodiments, the hair care composition can further comprise one or more conditioning agents, including cationic surfactants, cationic polymers and mixtures thereof. The term “cationic surfactant” means a surfactant that is positively charged when it is contained in the composition described herein. Suitable cationic surfactants can be chosen from primary, secondary or tertiary fatty amines, optionally polyoxyalkylenated, or salts thereof, and quaternary ammonium salts, and mixtures thereof. An exemplary fatty amine is stearamidopropyl dimethylamine. Exemplary quaternary ammonium salts include tetraalkylammonium salts, including dialkyldimethylammonium or alkyltrimethylammonium salts in which the alkyl group contains approximately from 16 to 22 carbon atoms, in particular behenyltrimethylammonium, di stearyldimethylammonium, cetyltrimethylammonium or benzyldimethylstearylammonium salts, or, on the other hand, the palmitylamidopropyltrimethylammonium salt, the stearamidopropyltrimethylammonium salt, the stearamidopropyldimethylcetearylammonium salt, or the stearamidopropyldimethyl(myristyl acetate)ammonium salt sold under the name CERAPHYL 70 by the company Van Dyk.

The term “cationic polymer” means any polymer containing cationic groups and/or groups that can be ionized to cationic groups, which can be non-siliceous. Exemplary cationic polymers include any known for styling the hair, for example, those described in patent application EP-A-0 337 354 and in French patent applications FR-A-2 270 846, 2 383 660, 2 598 611, 2 470 596 and 2 519 863, each of which is incorporated by reference in its entirety. Additional exemplary cationic polymers include those containing units comprising primary, secondary, tertiary and/or quaternary amine groups that may either form part of the main polymer chain or may be borne by a side substituent directly connected thereto. Suitable cationic polymers can have a weight-average molecular mass of greater than 105, including polymers of polyamine, polyaminoamide and polyquaternary ammonium type, including those described in French patents 2 505 348 and 2 542 997, each of which is incorporated by reference in its entirety.

In some embodiments, the hair care composition can further comprise a “non-sulfate” cleansing agent, lathering agent, or surfactant agent. Suitable “non-sulfate” agents include but are not limited to: sodium lauroyl methyl isethionate propanediol, sodium methyl oleoyl taurate, and sodium cocoyl isethionate.

In some embodiments, the hair care composition can further comprise any of the following ingredients or mixtures thereof: quaternary ammonium compound synthetically derived from rapeseed, quaternary polymers of vinylpyrrolidone and/or of vinylimidazole, for instance the products sold under the names LUVIQUAT FC 905, FC 550 and FC 370 and LUVIQUAT Excellence by the company BASF, Cationic polysaccharides, including cationic celluloses, including cellulose ether derivatives comprising quaternary ammonium groups, cationic cellulose copolymers or cellulose derivatives grafted with a water-soluble quaternary ammonium monomer, and cationic galactomannan gums. Exemplary cellulose ether derivatives comprising quaternary ammonium groups are described in French patent 1 492 597. These polymers are also defined in the CTFA dictionary as quaternary ammoniums of hydroxyethylcellulose that have reacted with an epoxide substituted with a trimethylammonium group. Cationic cellulose copolymers or the cellulose derivatives grafted with a water-soluble quaternary ammonium monomer are described in U.S. Pat. No. 4,131,576, such as hydroxyalkyl celluloses, for instance hydroxymethyl, hydroxyethyl or hydroxypropyl celluloses grafted especially with a methacryloylethyltrimethylammonium, methacrylamidopropyltrimethylammonium or dimethyldiallylammonium salt. Suitable associative celluloses such as alkylhydroxyethylcelluloses quaternized with C8-C30 fatty chains, such as the product QUATRISOFT LM 200, sold by the company Amerchol/Dow Chemical (INCI name Polyquaternium-24) and the products CRODACEL QM (INCI name PG-Hydroxyethylcellulose cocodimonium chloride), CRODACEL QL (C12 alkyl) (INCI name PG-Hydroxyethylcellulose lauryldimonium chloride) and CRODACEL QS (C18 alkyl) (INCI name PG-Hydroxyethylcellulose stearyldimonium chloride) sold by the company Croda. Other suitable fatty-chain hydroxyethylcellulose derivatives include the commercial products SOFTCAT Polymer SL such as SL-100, SL-60, SL-30 and SL-5 from the company Amerchol/Dow chemical of INCI name Polyquaternium-67. Suitable cationic galactomannan gums are described in U.S. Pat. No. 3,589,578 and 4 031 307. Suitable cellulosic ingredients can be in a crystalline form, a microcrystalline form, or a mixture thereof.

In some embodiments, the hair care composition can further comprise one or more cationic proteins or cationic protein hydrolysates, polyalkyleneimines, including polyethyleneimines, polymers containing vinylpyridine or vinylpyridinium units, condensates of polyamines and of epichlorohydrin, quaternary polyureylenes and chitin derivatives, animal protein hydrolysates bearing trimethylbenzylammonium groups, such as the products sold under the name Crotein BTA by the company Croda and referred to in the CTFA dictionary as Benzyltrimonium hydrolyzed animal protein, protein hydrolysates bearing quaternary ammonium groups on the polypeptide chain, the said ammonium groups comprising at least one alkyl radical having from 1 to 18 carbon atoms.

In some embodiments, the hair care composition can further comprise one or more quaternized plant proteins such as from wheat, corn or soybean proteins, for example, quaternized wheat proteins, including those sold by the company Croda under the names Hydrotriticum WQ or QM, referred to in the CTFA dictionary as Cocodimonium hydrolysed wheat protein, Hydrotriticum QL, referred to in the CTFA dictionary as Laurdimonium hydrolysed wheat protein, or else Hydrotriticum QS, referred to in the CTFA dictionary as Steardimonium hydrolysed wheat protein.

In some embodiments, the hair care composition can further comprise one or more polyamines such as POLYQUART R H sold by Cognis, referred to under the name polyethylene glycol tallow polyamine in the CTFA dictionary. Additional suitable polymers include those sold especially under the name Lupamin by the company BASF, and the products sold under the names Lupamin 9095, Lupamin 5095, Lupamin 1095, Lupamin 9030 and Lupamin 9010.

In some embodiments, the hair care composition can further comprise one or more fatty substances that are liquid at room temperature (25° C.) and at atmospheric pressure (1.013*105 Pa). The term “fatty substance” means an organic compound that is insoluble in water at ordinary temperature (25° C.) and at atmospheric pressure (1.013*105 Pa) (solubility of less than 5%, less than 1%, or less than 0.1%). Fatty substances are generally soluble in organic solvents under the same temperature and pressure conditions, for example, in chloroform, dichloromethane, carbon tetrachloride, ethanol, benzene, toluene, tetrahydrofuran (THF), liquid petroleum jelly or decamethylcyclopentasiloxane. The liquid fatty substances of the present disclosure can be nonpolyoxyethylenated and nonpolyglycerolated. The term “oil” means a “fatty substance” that is liquid at room temperature (25° C.) and at atmospheric pressure (1.013*105 Pa). The term “non-silicone oil” means an oil not containing any silicon atoms (Si) and the term “silicone oil” means an oil containing at least one silicon atom. The liquid fatty substances can be chosen from non-silicone oils such as in particular C6-C16 liquid hydrocarbons, liquid hydrocarbons containing more than 16 carbon atoms, non-silicone oils of animal origin, triglycerides of plant or synthetic origin, fluoro oils, liquid fatty acid and/or fatty alcohol esters other than triglycerides, and mixtures thereof. The liquid hydrocarbons can be linear, branched or optionally cyclic, including hexane, cyclohexane, undecane, dodecane, tridecane or isoparaffins, such as isohexadecane, isodecane or isododecane, and mixtures thereof. Suitable linear or branched liquid hydrocarbons of mineral or synthetic origin containing more than 16 carbon atoms can be chosen from liquid paraffins, petroleum jelly, liquid petroleum jelly, mineral oil, polydecenes and hydrogenated polyisobutene such as PARLEAM and mixtures thereof. Hydrocarbon-based oils of animal origin, such as perhydrosqualene, can be used.

Exemplary triglycerides of vegetable or synthetic origin can be chosen from liquid fatty acid triglycerides comprising from 6 to 30 carbon atoms, for instance heptanoic or octanoic acid triglycerides, or alternatively, more particularly from those present in plant oils, for instance coconut oil, sunflower oil, corn oil, soybean oil, marrow oil, grapeseed oil, sesame seed oil, hazelnut oil, apricot oil, macadamia oil, arara oil, castor oil, avocado oil, jojoba oil, shea butter oil or synthetic caprylic/capric acid triglycerides, such as those sold by the company Stearineries Dubois or those sold under the names MIGLYOL 810, 812 and 818 by the company Dynamit Nobel, and mixtures thereof. Suitable fluoro oils include perfluoromethylcyclopentane and perfluoro-1,3-dimethylcyclohexane, such as FLUTEC PC1 and FLUTEC PC3 by the company BNFL Fluorochemicals; perfluoro-1,2-dimethylcyclobutane; perfluoroalkanes such as dodecafluoropentane and tetradecafluorohexane, sold under the names PF 5050 and PF 5060 by the company 3M, or bromoperfluorooctyl sold under the name FORALKYL by the company Atochem; nonafluoromethoxybutane and nonafluoroethoxyisobutane; perfluoromorpholine derivatives such as 4-trifluoromethyl perfluoromorpholine sold under the name PF 5052 by the company 3M.

Suitable monoesters include dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate, and mixtures thereof.

In some embodiments, the hair care composition can further comprise diethyl sebacate, diisopropyl sebacate, diisopropyl adipate, di(n-propyl) adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and polyethylene glycol distearates, and mixtures thereof.

In some embodiments, the hair care composition can further comprise one or more fatty esters, one or more sugar esters, and/or one or more diesters of C6-C30, such as C12-C22 fatty acids. The term “sugar” means oxygen-bearing hydrocarbon-based compounds containing several alcohol functions, with or without aldehyde or ketone functions, and which comprise at least 4 carbon atoms. Suitables sugars can include monosaccharides, oligosaccharides or polysaccharides, for example, sucrose (or saccharose), glucose, galactose, ribose, fucose, maltose, fructose, mannose, arabinose, xylose and lactose, and derivatives thereof, such as alkyl derivatives, such as methyl derivatives, for instance methylglucose. Suitable esters can include oleates, laurates, palmitates, myristates, behenates, cocoates, stearates, linoleates, linolenates, caprates, arachidonates or mixtures thereof, such as, oleate/palmitate, oleate/stearate or palmitate/stearate mixed esters. Suitable monoesters and diesters also include mono- or di-oleate, -stearate, -behenate, -oleopalmitate, -linoleate, -linolenate or -oleostearate of sucrose, of glucose or of methylglucose, including the product sold under the name GLUCATE DO by the company Amerchol, which is a methylglucose dioleate. Additional exemplary esters or mixtures of esters of sugar and of fatty acid that may also be mentioned include: the products sold under the names F160, F140, F110, F90, F70 and SL40 by the company Crodesta, respectively denoting sucrose palmitate/stearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose mono laurate; the products sold under the name Ryoto Sugar Esters, for example reference B370 and corresponding to sucrose behenate formed from 20% monoester and 80% diester-triester-polyester; the sucrose mono-dipalmitate/stearate sold by the company Goldschmidt under the name TEGOSOFT PSE.

In some embodiments, the hair care composition can further comprise a pH modifying agent such as citric acid and/or sodium hydroxide. Any commonly used pH-modifying agent for hair care compositions is contemplated for use herein.

In various embodiments, the hair care composition can further comprise any of the following ingredients, and/or mixtures thereof: sodium lauroyl methyl isethionate (a cleansing and lathering agent), liquid fatty substances including silicone oils different from the non-amino polyalkylsiloxanes discussed above herein, and organomodified polysiloxanes comprising at least one functional group chosen from amino groups, aryl groups and alkoxy groups. Organopolysiloxanes are defined in greater detail in Walter Noll's Chemistry and Technology of Silicones (1968), Academic Press, the entirety of which is hereby incorporated by reference. They may be volatile or non-volatile. Suitable cyclic polydialkylsiloxanes include octamethylcyclotetrasiloxane sold under the name VOLATILE SILICONE 7207 by Union Carbide or SILBIONE 70045 V2 by Rhodia, decamethylcyclopentasiloxane sold under the name VOLATILE SILICONE 7158 by Union Carbide, and SILBIONE 70045 V5 by Rhodia, and mixtures thereof. Cyclocopolymers of the dimethylsiloxane/methylalkylsiloxane type, such as VOLATILE SILICONE FZ 3109 sold by the company Union Carbide are also suitable. Exemplary cyclic polydialkylsiloxanes with organosilicon compounds include octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane, linear volatile polydialkylsiloxanes such as those sold under the name SH 200 by the company Toray Silicone. Silicones coming within this category are also described in the paper published in Cosmetics and Toiletries, Vol. 91, January 76, pp. 27-32, Todd & Byers, “Volatile Silicone Fluids for Cosmetics,” which is incorporated by reference herein.

Exemplary suitable non-volatile polydialkylsiloxanes include polydimethylsiloxanes having trimethylsilyl end groups such as the SILBIONE oils of the 47 and 70 047 series or the MIRASIL oils sold by Rhodia, such as, for example, the oil 70 047 V 500 000; the oils of the MIRASIL series sold by Rhodia; the oils of the 200 series from the company Dow Corning, such as DC200 with a viscosity of 60 000 mm2/s; the VISCASIL oils from General Electric and certain oils of the SF series (SF 96, SF 18) from General Electric, the polydimethylsiloxanes having dimethylsilanol end groups known under the name dimethiconol (CTFA), such as the oils of series 48 from the company Rhodia.

Exemplary organomodified silicones include polyalkylarylsiloxanes, and products sold under the following names: SILBIONE oils of the 70 641 series from Rhodia; the oils of the RHODORSIL 70 633 and 763 series from Rhodia; the oil Dow Corning 556 Cosmetic Grade Fluid from Dow Corning; the silicones of the PK series from Bayer, such as the product PK20; the silicones of the PN and PH series from Bayer, such as the products PN1000 and PH1000, certain oils of the SF series from General Electric, such as SF 1023, SF 1154, SF 1250 and SF 1265, the products sold under the names GP 4 Silicone Fluid and GP 7100 by Genesee or the products sold under the names Q2 8220 and Dow Corning 929 or 939 by the company Dow Corning.

In various embodiments, the hair care composition can further comprise one or more additional surfactants different from the cationic surfactants described above, including anionic surfactants, amphoteric or zwitterionic surfactants, nonionic surfactants and mixtures thereof. Exemplary anionic surfactants include alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkyl sulfosuccinates, alkylether sulfosuccinates, alkylamide sulfo succinates, alkylsulfoacetates, acylsarcosinates, acylglutamates, alkyl sulfosuccinamates, acylisethionates and N--(C1-C4)alkyl N-acyltaurates, salts of alkyl monoesters and of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds (unless otherwise mentioned) generally comprising from 6 to 24 carbon atoms and the aryl group generally denoting a phenyl group. Anionic surfactants in salt form can include alkali metal salts such as the sodium or potassium salt, the sodium salt, ammonium salts, amine salts and amino alcohol salts or alkaline-earth metal salts such as the magnesium salt. Exemplary amino alcohol salts include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Suitable anionic surfactants also include mild anionic surfactants, i.e. anionic surfactants without a sulfate function, including polyoxyalkylenated alkyl ether carboxylic acids; polyoxyalkylenated alkylaryl ether carboxylic acids; polyoxyalkylenated alkylamido ether carboxylic acids, in particular those comprising 2 to 50 ethylene oxide groups; alkyl-D-galactoside uronic acids; acylsarcosinates, acylglutamates; and alkylpolyglycoside carboxylic esters such as those sold under the name AKYPO RLM 45 CA from Kao.

Exemplary suitable amphoteric or zwitterionic surfactant(s) can be secondary or tertiary aliphatic amine derivatives, optionally quaternized, in which the aliphatic group is a linear or branched chain containing from 8 to 22 carbon atoms, where the amine derivatives contain at least one anionic group, for example a carboxylate, sulfonate, sulfate, phosphate or phosphonate group, such as (C8-C20)alkylbetaines, sulfobetaines, (C8-C20)alkylamido(C3-C8)alkylbetaines or (C8-C20)alkylamido(C6-C8)alkylsulfobetaines. Any suitable secondary or tertiary aliphatic amine derivative can be present in coconut oil or in hydrolysed linseed oil, or the like. Representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, the cocoamphodiacetate sold by the company Rhodia under the trade name MIRANOL C2M Concentrate, and sodium diethylaminopropyl cocoaspartamide and sold by the company Chimex under the name Chimexane HB are suitable for use in the disclosed compositions.

Suitable nonionic surfactants are described in the Handbook of Surfactants by M. R. Porter, published by Blackie & Son (Glasgow and London), 1991, pp. 116-178, and include fatty alcohols, fatty α-diols, fatty (C1-C20)alkylphenols and fatty acids, which can be ethoxylated, propoxylated or glycerolated and contain at least one fatty chain comprising from 8 to 18 carbon atoms, the number of ethylene oxide or propylene oxide groups ranging from 1 to 200, and the number of glycerol groups ranging from 1 to 30. Condensates of ethylene oxide and of propylene oxide with fatty alcohols, ethoxylated fatty amides having from 1 to 30 ethylene oxide units, polyglycerolated fatty amides comprising on average from 1 to 5 glycerol groups, and in particular from 1.5 to 4, ethoxylated fatty acid esters of sorbitan containing from 1 to 30 ethylene oxide units, fatty acid esters of sucrose, fatty acid esters of polyethylene glycol, (C6-C24)alkylpolyglycosides, oxyethylenated plant oils, N—(C6-C24)alkylglucamine derivatives, amine oxides such as (C10-C14)alkylamine oxides or N—(C10-C14)acylaminopropylmorpholine oxides are also suitable for use in the compositions disclosed herein.

Additional suitable nonionic surfactants include alkyl polyglucosides (APG), maltose esters, polyglycerolated fatty alcohols, glucamine derivatives, for instance 2-ethylhexyloxycarbonyl-N-methylglucamine, and mixtures thereof. Alkylpolyglucosides that are those containing an alkyl group comprising from 6 to 30 carbon atoms, and containing a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UPg by the company Cognis.

Suitable maltose derivatives include those described in document EP-A-566 438, such as O-octanoyl-6′-D-maltose or O-dodecanoyl-6′-D-maltose described in document FR-2 739 556. Each of these documents is incorporated by reference in its entirety.

In some embodiments, the hair care composition can be formulated in a cosmetically acceptable medium. The term “cosmetically acceptable medium” means a medium that is compatible with human keratin fibers, such as the hair. A cosmetically acceptable medium can be formed from water or from a mixture of water and one or more cosmetically acceptable solvents chosen from lower alcohols, such as ethanol and isopropanol; polyols and polyol ethers, including 2-butoxyethanol, propylene glycol, propylene glycol monomethyl ether, diethylene glycol monoethyl ether and monomethyl ether, and mixtures thereof.

In some embodiments, the hair care composition can further comprise any of the following additives, or mixtures thereof: solid fatty substances different from fatty alcohols such as waxes, anionic, nonionic or amphoteric polymers or mixtures thereof, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners different from oxyethylenated polymers, opacifiers or nacreous agents, antioxidants, hydroxy acids, fragrances and/or preserving agents.

Nail Care

In some embodiments, the cosmetic composition can comprise ingredients commonly used in nail care products. Nail care products include, but are not limited to, nail treatments including nail strengtheners, top coats, and base coats, nail polishes, nail polish removers, hands skincare, feet skincare, drying agents, and corrector pens including nail polish removers.

In some embodiments, the nail care composition can be a nail treatment composition. A nail treatment composition can comprise a composition for treating ingrown nails or nail deformities, a composition for topical treatment of nail infections, including fungal infections, nail strengtheners, top coats, base coats, polish removers, or any combination thereof. In some embodiments, a nail treatment composition can be formulated as a topical nail lacquer or polish, creams, solutions, suspensions, lotions, serums, gels, balms, gels, oils, oil in creams, and/or scrubs for treating the hands and/or the feet.

In some embodiments, the nail treatment composition can be nail strengthener. Nail strengtheners can treat fingernails and toenails to both harden, strengthen, and promote growth of the nails, to prevent or minimize breaking, cracking, splitting and peeling, and can comprise any known composition used to prevent and heal quarter cracks while increasing the growth of horse hooves, which have a similar protein consistency to human fingernails and toenails, including: lanolin, butter, beeswax, rosin, copper acetate, and turpentine. In some embodiments, the nail treatment composition can further comprise titanium Dioxide for example, TI-PURE R900 from E.I. DuPont.

In some embodiments, the nail treatment composition can be a base coat. In some embodiments, a base coat can be a liquid composition comprising at least one polymer which provides adhesion, for example a polymer co-polymerized from methyl methacrylate (MMA) and methacrylic acid (MAA) to form a polymer composed of polymethyl methacrylate (PMMA) and polymethacrylic acid (PMAA), in which the MAA monomer fraction may vary from 0 to 100%. In some embodiments, suitable polymers for use as a base coat include hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, and mixtures thereof. In some aspects, a base coat can further comprise a non-reactive, solvent-dissolvable, film-forming polymer such as a cellulose ester, for example cellulose acetate alkylate, cellulose acetate butyrate, or cellulose acetate propionate. The above exemplary ingredients are not limiting.

In some embodiments, the nail treatment composition can be a top coat, for example a quick drying top coat. Top coats can include a base resin which is cellulose acetate butyrate, a film former which is a methacrylate polymer, thermally curable or photocurable monomers which are monofunctional methacrylates and cross-linkers (i.e., difunctional and trifunctional methacrylates), a thermal initiator or photoinitiator(s) together in a solution of aliphatic esters and alcohol, and a photoreactive coating. Suitable solvents include acetates and alcohols, particularly ethyl acetate, butyl acetate, and isopropyl alcohol. The photoreactive coating can include photoreactive monomers including methacrylate monomers such as: cyclohexyl methacrylate, n-decyl methacrylate, 2-ethyl hexyl methacrylate, ethyl methacrylate, hydroxy propyl methacrylate, isobornyl methacrylate, 2-methoxy ethyl methacrylate; difunctional and trifunctional methacrylate monomers, cross-linking agents such as diurethane dimethacrylate, ethylene glycol dimethacrylate, 1,10 decanediol dimethacrylate, 1,6-hexanediol dimethacrylate, and trimethylolpropane trimethacrylate. Commercially available photoinitiators suitable for use include but are not limited to: benzoin methyl ether, 2-hydroxy-2-methyl-1-phenyl-1-propanone (“Darocur 1173”), diethoxyacetophenone, and benzyl diketal. Photoinitiators are oligomeric mixtures of phenyl propanones such as a mixture of 2,4,6-trimethylbenzophenone and 4-methylbenzophenone and a mixture of oligo-[2-hydroxy-2-methyl-1[4-(1-methylvinyl)phenyl]propanone] and 2-hydroxy-2-methyl-phenyl propanone that are sold by Sartomer under the names “Esacure KIP 100F” and “Esacure TZT Photoinitiator.”

In some embodiments, the nail care composition can be a nail polish or nail enamel composition. In some embodiments, the nail polish or nail enamel composition can further comprise a nitrocellulose film former, a latex film former, a polycarbodiimide film former, a low volatile organic compound (VOC), and polycarbodiimide film formers. Polycarbodiimides can include polymers with a plurality of carbodiimide groups appended to the polymer backbone. For example, U.S. Pat. No. 5,352,400 (the disclosure of which is incorporated by reference herein) discloses polymers and co-polymers derived from alpha-methylstyryl-isocyanates. Suitable polycarbodiimide compounds include, but are not limited to, those commercially sold by the suppliers Nisshinbo (including those known by the name under the CARBODILITE series, V-02, V02-L2, SV-02, E-02, V-10, SW-12G, E-03A), Picassian, and 3M.

In some embodiments, the nail polish or nail enamel composition can further comprise one or more latex polymers, including carboxyl functional acrylate latex polymers, carboxyl functional polyurethane latex polymers, carboxyl functional silicone latex polymers, carboxyl functional non-acrylate latex polymers and mixtures thereof. In various embodiments, suitable latex polymers can be film-forming latex polymers or non film-forming latex polymers. In some embodiments, the latex polymers can be carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers The term “(meth)acryl” and variations thereof, as used herein, means acryl or methacryl. The (meth)acrylic monomers may be chosen from, for example, acrylic acid, methacrylic acid, citraconic acid, itaconic acid, maleic acid, fumaric acid, crotonic acid, and maleic anhydride. The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N--(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, .alpha.-methylstyrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers herein is not limiting, and it should be understood that it is possible to use any monomer known to those skilled in the art which includes acrylic and/or vinyl monomers (including monomers modified with a silicone chain).

In some non-limiting exemplary embodiments, carboxyl functional acrylate latex polymers can be chosen from aqueous dispersions of Methacrylic Acid/Ethyl Acrylate copolymer (INCI: Acrylates Copolymer, such as LUVIFLEX SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), Acrylates Copolymers, such as those known under the tradenameDermacryl AQF (Akzo Nobel), under the tradename LUVIMER MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In some embodiments, the nail care composition can comprise a nail polish remover. Nail polish removers can include a polyhydric alcohol compound including glycerin, glycols, polyglycerin, esters of polyhydric alcohols, and mixtures thereof. The glycol can contain from 2 to 12 carbon atoms, such as, for example, glycerin, propylene glycol, butylene glycol, propane diol, hexylene glycol, polyglycerin, dipropylene glycol and diethylene glycol. Suitable esters of polyhydric alcohol include liquid esters of saturated or unsaturated, linear or branched C1-C26 polyhydric alcohols. Examples of suitable esters of polyhydric alcohol include, but are not limited to, esters of dihydroxy, trihydroxy, tetrahydroxy or pentahydroxy alcohols. The ester of polyhydric alcohol may be a glyceryl ester such as, glyceryl triglycolate, glyceryl tricitrate, glyceryl trilactate, glyceryl trilactate, glyceryl tributanoate, glyceryl triheptanoate, glyceryl trioctanoate, etc.

A nail polish remover can further comprise a low carbon alcohol, a containing from 1 to 8 carbon atoms. The low carbon alcohol may contain from 2 to 6 carbon atoms, such as from 2 to 5 carbon atoms. Examples of low carbon alcohols include, but are not limited to, ethanol, propanol, butanol, pentanol, isopropanol, isobutanol, and isopentanol. A nail polish remover can further comprise a high boiling point ester compound including, but are not limited to, carbonate esters, adipates, sebacates and succinates. Exemplary high boiling point ester compounds include, but are not limited to, alkylene carbonates such as propylene carbonate, dimethyl succinate, diethyl succinate, dimethyl glutarate, diethyl glutarate, dimethyl sebacate, diethyl sebacate, diisopropyl sebacate, bis(2-ethylhexyl) sebacate, dimethyl adipate, diisopropyl adipate, di-n-propyl adipate, dioctyl adipate, bis(2-ethylhexyl) adipate, diisostearyl adipate, ethyl maleate, bis(2-ethylhexyl) maleate, triisopropyl citrate, triisocetyl citrate, triisostearyl citrate, trioctyldodecyl citrate and trioleyl citrate.

A nail polish remover can further comprise a thickening agent including but not limited to: nonionic, anionic, cationic, amphiphilic, and amphoteric polymers, and other known rheology modifiers, such as cellulose-based thickeners such as hydroxyethylcellulose, hydroxypropylcellulose, methylcellulose, and ethylhydroxyethylcellulose. Certain notable cellulose derivatives include hydroxyl-modified cellulose polymers such as Hydroxyethylcellulose, e.g., those having a molecular weight over 500,000 daltons such as NATROSOL 250 HHR and Hydroxypropyl cellulose, e.g., KLUCEL MF—both available from Ashland of Covington, Ky. The thickening agent can be a polysaccharide such as fructans, glucans, galactans and mannans or heteropolysaccharides such as hemicellulose, pullulan or branched polysaccharides such as gum arabic and amylopectin, or mixed polysaccharides such as starch. The thickening agent can be an acrylic thickening agent (acrylic thickener) or an acrylamide thickening agent (acrylamide thickener). The thickening agent can comprise at least one monomer performing a weak acid function such as acrylic acid, methacrylic acid, itaconic acid, crotonic acid, maleic acid and/or fumaric acid. The thickening agent can comprise a monomer performing a strong acid function such as monomers having a function of the sulfonic acid type or phosphonic acid type, such as 2-acrylamido-2-methylpropane sulfonic acid (AMPS). The thickening agent can comprise a crosslinking agent such as methylene bisacrylamide (MBA), ethylene glycol diacrylate, polyethylene glycol dimethacrylate, diacrylamide, cyanomethacrylate, vi nyloxyethacrylate or methacrylate, formaldehyde, glyoxal, and compositions of the glycidylether type such as ethyleneglycol diglycidylether, or epoxides. Suitable acrylic thickeners are disclosed in U.S. patent application publication nos. 2004/0028637 and 2008/0196174, both of which are incorporated herein by reference. In some embodiments, the thickening agent can comprise an organoclay (hydrophobically treated clay) or a hydrophilic clay.

In some embodiments, the thickening agent can comprise an abrasive compound (abrasive system). An “abrasive compound” is a compound capable of providing abrasion or mechanical exfoliation. The abrasive particles can comprise perlite, pumice, zeolites, hydrated silica, calcium carbonate, dicalcium phosphate dihydrate, calcium pyrophosphate, alumina, sodium bicarbonate, polylactic acid, as well as synthetic polymeric materials such as polyethylene, polypropylene, polyethylene terephthalate, polymethlyl methacrylate or nylon. In certain embodiments a moderately hard abrasive includes perlite, such as a cosmetic grade perlite available from Imerys under the name IMERCARE 270P-Scrub. In certain embodiments a soft abrasive is a sugar, a ground fruit kernel or shell powders such as apricot kernel, coconut husk, or spherical waxes (for example, carnauba jojoba); argan shell powder, and the like.

In some embodiments, the nail care composition can further comprise an additive commonly used in cosmetic compositions and known to a person skilled include solvents, preservatives, fragrances, oils, waxes, surfactants, antioxidants, agents for combating free radicals, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

d. Therapeutic Compositions

In some embodiments, the composition described herein can be a therapeutic composition that is useful for treating one or more conditions. For example, and in some embodiments, application of the compositions described herein can promote wound healing, reduce or prevent the formation of scar tissue, promote tissue regeneration, minimize local inflammation, minimize tissue rejection, and/or enhance graft integration. In some embodiments, the composition can be formulated as an injectable material, e.g., a hydrogel. Collagen hydrogels present a large, uniform surface area, and can serve as a delivery system for collagen and, optionally, one or more additional therapeutic agents. Injectable collagen materials can also form scaffolds or networks capable of both replacing tissue function and supporting tissue regeneration. In certain embodiments, the composition can be topically applied. In certain embodiments, the composition can be dermally, intradermally, or subcutaneously injected.

In some embodiments, the therapeutic compositions described herein can comprise one or more additional therapeutic agents and/or prophylactic agents—other than the collagen fragments described elsewhere herein. The one or more additional therapeutic and/or prophylactic agents can be a small molecule active agent or a biomolecule, such as an enzyme or protein, polypeptide, or nucleic acid.

Non-limiting examples of additional therapeutic and/or prophylactic agents include anti-cancer agents, antimicrobial agents (including anti-viral agents, antibacterial agents, anti-fungal agents, and anti-parasitic agents), antioxidants, analgesics, local anesthetics, anti-inflammatory agents, cytokines, immunosuppressant agents, anti-allergenic agents, essential nutrients, growth factors (such as fibroblast growth factor, hepatocyte growth factor, platelet-derived growth factor, vascular endothelial cell growth factor, and insulin-like growth factor), and combinations thereof. Specific dosages of the additional therapeutic and/or prophylactic agents can be readily determined by those of skill in the art. See Ansel, Howard C. et al., Pharmaceutical Dosage Forms and Drug Delivery Systems (6th ed.) Williams and Wilkins, Malvern, PA (1995).

In other embodiments, the recombinant collagen fragment composition described herein can be used in combination with cell delivery, for example, the delivery of stem cells, pluripotent cells, somatic cells, and combinations thereof.

Although the present disclosure contemplates that the therapeutic and/or prophylactic agents will be an integral part of the composition described herein, in some embodiments, the therapeutic and/or prophylactic agents can be administered prior to, in conjunction with, or subsequent to administration of the therapeutic composition described herein. In other words, in some embodiments, the one or more additional therapeutic and/or prophylactic agents may not be integrally incorporated into the present compositions, but be provided separately from the present compositions.

Non-limiting examples of suitable local anesthetics capable of being included in the present compositions include, but are not limited to, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxy tetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof.

Non-limiting examples of suitable antiviral agents include ganciclovir and acyclovir. Non-limiting examples of suitable antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines. Additional exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

Non-limiting examples of suitable anti-inflammatory agents include steroidal active agents include glucocorticoids, progestins, mineralocorticoids, and corticosteroids. Exemplary non-steroidal anti-inflammatory drug include ketorolac, ibuprofen, nepafenac, diclofenac, aspirin, and naproxen. Other exemplary anti-inflammatory agents include triamcinolone acetonide, fluocinolone acetonide, prednisolone, dexamethasone, loteprendol, fluorometholone, and dipotassium glycyrrhizate.

In some embodiments, the composition can further comprise one or more additional pharmaceutically active agents. Exemplary pharmaceutical agents can include non-steroidal anti-inflammatory agents (NSAIDs), e.g., flurbiprofen, ibuprofen, naproxen, indomethacin and related compounds. In some embodiments, the composition can further comprise one or more anti-mitotic drugs including colchicine, taxol and related compounds. In some embodiments, the composition can further comprise one or more topical antiseptics such as, e.g., benzoyl peroxide. In some embodiments, the composition can further comprise one or more polysaccharides produced by microalgae, e.g., alguronic acid.

In some embodiments, the composition can further comprise one or more immune-modulating drugs. Exemplary immune-modulating drugs include imiquimod, cyclosporine, tacrolimus, and rapamycin.

In some embodiments, the composition can further comprise one or more cytokines. Exemplary suitable cytokines include, but are not limited to, IL-10, TGF-β, IL-25, and IL-35. In certain embodiments, the cytokines can induce Treg activation (e.g. IL-25) and suppress Th17 activation (e.g. IL-10) in order to minimize rejection.

In some embodiments, the compositions described herein further comprises at least one eukaryotic cell type. Some exemplary eukaryotic cell types include stem cells, mesenchymal stem cells, keratinocytes, fibroblasts, melanocytes, adipocytes, immune cells such as T lymphocytes, B lymphocytes, natural killer cells, and dendritic cells, or combinations thereof. In some embodiments, the stem cells can be adipose-derived mesenchymal stem cells. Functional characteristics of mesenchymal stem cells that can benefit wound healing include their ability to migrate to the site of injury or inflammation, participate in regeneration of damaged tissues, stimulate proliferation and differentiation of resident progenitor cells, promote recovery of injured cells through growth factor secretion and matrix remodeling, and exert unique immunomodulatory and anti-inflammatory effects (See e.g., Phinney O G et al., Stem Cells, 25:2896-2902 (2007); Chamberlain G et al, Stem Cells, 25:2739-2749(2007); Dazzi F et al., Curr Opin Oncol. 19:650-655 (2007)). Each of these references is incorporated by reference in its entirety.

In some embodiments, the eukaryotic cell can be responsible for increasing the structural integrity of connective tissue and/or promote healing. In some embodiments, the eukaryotic cell, such as a fibroblast, can be responsible for enhancing or promoting the growth or connection of cells or tissues.

In some embodiments, the recombinant collagen composition can be in contact with 1) an implanted hair graft and 2) tissue into which the hair graft is implanted. Recipient sites for hair grafts include the scalp, the facial region, the armpit or chest region or the pubic region. Specific areas of the facial region include eyebrows, eyelid, mustache, sideburn, chin and cheeks. The recipient site can be any area of skin wherein the appearance of hair is desired by the subject. In some embodiments, the contact between the composition described herein and the implanted hair with the surrounding tissues of the recipient site promotes nutritional perfusion from the surrounding tissues into the graft and increases the survival rate of the graft compared to a graft implanted without the use of the composition described herein. In certain embodiments, the contact between the composition described herein and the implanted hair, and between the composition described herein and the surrounding tissues of the recipient site promotes vascularization around the implanted grafts, such that the survival rate of the graft is increased compared to a graft implanted without the use of the composition described herein.

e. Dietary Compositions

In some embodiments, the composition described herein can be a dietary composition useful for useful for providing collagen to a subject in need thereof. For example, and in some embodiments, consumption of the dietary compositions described herein can provide health and/or skin benefits, such as increasing collagen intake, relieving joint pain, and improving skin health. In certain embodiments, the composition can be in the form of a powder, a capsule, a liquid, or any other suitable form.

In some embodiments, the dietary compositions can comprise one or more nutritional ingredients such as: ascorbic acid, biotin, chromium nicotinate, copper citrate, D-calcium pantothenate, cyanocobalamin, flax seed, Linum usitatissimum, folic acid, fructooligosaccharide (fiber), magnesium oxide, manganese citrate, maltodextrin, medium chain triglycerides, flavor, niacinamide, potassium citrate potassium iodide, riboflavin, sugar cane (Saccharum officinarum), sodium molybdate dihydrate, sodium selenate (selenium), soy protein isolate, stevia leaf extract/Stevia rebaudiana, thiamin HCl, tricalcium phosphate, vitamin a palmitate, vitamin D3, xanthan gum, zinc citrate, cellulose gum, guar gum, pyridoxine hydrochloride, salt, tocopherol, antioxidants, e.g., resveratrol, CoQ10, acai berry, lycopene and pomegranate, natural or artificial sweeteners, e.g., glucose, sucrose, fructose, saccharides, cyclamates, aspartamine, sucralose, aspartame, acesulfame K, or sorbitol, flavoring, such as a flavored extract, volatile oil, chocolate flavoring (e.g., non-caffeinated cocoa or chocolate, chocolate substitutes such as carob), peanut butter flavoring, cookie crumb, vanilla, or any commercially available flavoring, and any combination thereof.

f. Methods of Producing Recombinant Collagen Fragments

In some embodiments, the recombinant collagen fragment described herein can be produced in a genetically engineered strain of yeast. Suitable yeast include those of the genus Pichia, Candida, Komatagaella, Hansenula, Cryptococcus, Saccharomyces and combinations thereof. In some embodiments, the yeast can be from the genus Pichia. The yeast can be modified or hybridized. Hybridized yeast can be prepared by breeding different strains of the same species, different species of the same genus, or strains of different genera. Examples of yeast strains that are suitable to produce the collagen fragments disclosed herein include Pichia pastoris, Pichia membranifaciens, Pichia deserticola, Pichia cephalocereana, Pichia eremophila, Pichia myanmarensis, Pichia anomala, Pichia nakasei, Pichia siamensis, Pichia heedii, Pichia barkeri, Pichia norvegensis, Pichia thermomethanolica, Pichia stipites, Pichia subpelliculosa, Pichia exigua, Pichia occidentalis, Pichia cactophila, and the like. In a particular embodiment, the yeast is Pichia pastoris.

In some embodiments, the method comprises (i) fermenting a genetically engineered yeast in a fermentation broth; (ii) recovering from the supernatant of the fermentation broth recombinant collagen fragments secreted by the genetically engineered yeast; and (iii) optionally, purifying the recombinant collagen fragments. In some embodiments, the method further comprises lyophilization.

In some embodiments, the method comprises (i) fermenting a genetically engineered yeast in a fermentation broth; (ii) recovering the intracellular recombinant collagen fragments which were not secreted into the supernatant of the fermentation broth by the genetically engineered yeast; and (iii) optionally, purifying the recombinant collagen fragments. In some embodiments, the recovering comprises recovering the intracellular recombinant collagen fragments from the whole fermentation broth. In some embodiments, the recovering comprises recovering the intracellular recombinant collagen fragments from a cell paste. In some embodiments, the method further comprises lyophilization.

g. Methods of Use

In some embodiments, the composition described herein can be a skincare composition that can be used to treat an area of the skin by topically applying the skincare composition to the area of skin.

The skincare composition described herein can be applied to any body surface, including, but not limited to, a facial surface, the scalp, neck, ears, shoulders, chest (including breasts and/or the décolletage), arms, hands, legs, stomach, buttocks, groin, back, feet, and combinations thereof. In some embodiments, the facial surface can be the forehead, eyes, a perioral surface, a chin surface, a periorbital surface, a nasal surface, a cheek skin surface, or a combination thereof.

In some embodiments, administration of the composition described herein to the skin can improve or maintain the quality of skin and reduce or eliminate signs of aging. Signs of aging include, but are not limited to, all outward visibly and tactilely perceptible manifestations as well as any other macro or micro effects due to skin aging. Such signs may be induced or caused by intrinsic factors or extrinsic factors (such as chronological aging and/or environmental damage). These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin.

In some embodiments, a composition as described herein can be suitable for use as a dermal filler. A dermal filler composition can replace lost endogenous matrix polymers, or enhance/facilitate the function of existing matrix polymers, in order to treat skin conditions due to aging or injury. The dermal filler composition can fill wrinkles, lines, folds, scars, and to enhance dermal tissue, such as, e.g., to plump thin lips, or fill-in sunken eyes or shallow cheeks. Earlier dermal filler products generally were made of collagens. One common matrix polymer used in modern dermal filler compositions is hyaluronan. Because hyaluronan is natural to the human body, it is a generally well tolerated and a fairly low risk treatment for a wide variety of skin conditions.

In some embodiments, a composition as described herein can suitable for use with a microneedle sheet or patch. Microneedles can provide the composition described herein to any area of the face and body to achieve a cosmetic benefit or wound healing benefit.

In some embodiments, the composition described herein can be a therapeutic composition that can be used to reduce or prevent the formation of scar tissue, promoting healing, promoting tissue regeneration, minimizing local inflammation, minimizing tissue rejection, and/or enhance skin and/or hair graft integration.

In some embodiments, the disclosure provides a method of treating a dermatological condition comprising administering an effective amount of the composition comprising a recombinant collagen fragment to a subject in need thereof. In some embodiments, the dermatological condition can be fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

In some embodiments, the composition can be topically administered to an area of skin. In some embodiments, the area of skin can be selected from the group consisting of a facial surface, scalp, neck, ears, shoulders, chest (including breasts and/or the décolletage), arms, hands, legs, stomach, buttocks, groin, back, feet, and combinations thereof.

In some embodiments, the composition can be topically administered to a facial surface. In some embodiments, the facial surface can be selected from the group consisting of forehead, eyes, a perioral surface, a chin surface, a periorbital surface, a nasal surface, a cheek skin surface, and combinations thereof.

In some embodiments, the disclosure provides a method for improving collagen production in the skin, comprising administering an effective amount of a composition comprising a recombinant collagen fragment.

In some embodiments, the disclosure provides a skincare product comprising the composition described herein for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

In some embodiments, the composition described herein can be a skincare composition that can be used to treat the skin, hair, and nails, by consuming the composition as a dietary supplement.

### EXAMPLES

### Example 1: Production of Permeate Concentrate

Pichia pastoris modified to express type III recombinant collagen were prepared as described in U.S. application Ser. No. 16/125,386 (issued as U.S. Pat. No. 11,028,146), U.S. application Ser. No. 16/048,920, and U.S. application Ser. No. 16/023,525, which are herein incorporated by reference in their entirety. The modified Pichia pastoris were cultured in BMGY media in a shake flask. The BMGY media was supplemented as shown in Table 1.

TABLE 1
Shake Flask Seed BMGY Media Composition

| Component | Use rate in BMGY medium |
|---|---|
| 1 L Sterile BMGY | 982 |
| 50 mg/mL Geneticin | 10 |
| 99% Glycerol | 10 |

50 mL of the prepared media inoculate containing modified Picha pastoris was transferred to a 250 mL baffled shake flask with a 0.22 μm PTFE vented cap link. 0.5 mL of sterile glycerol stock was added to the shake flask and the shake flask was cultivated at 32° C. and 350 rpm in an Infors HT Multitron Pro shaker with a shaker amplitude of 25 mm for between 23 and 26 hours, until reaching a target OD₆₀₀ of 15. Shake flask cultivate conditions are shown in Table 2.

TABLE 2
Shake Flask Seed Propagation Conditions

| Parameter | Value | |
|---|---|---|
| Shake flask volume per initial fermenter | 40-50 | mL/L |
| volume | | |
| Fermenter target initial OD | 0.75 | |
| Temperature | 32 | C. |
| Shaker speed | 350 | rpm |
| Shaker throw/amplitude | 25 | mm |

| | |
|---|---|
| Target final biomass density after ~23-26 h | 15 |
| (OD600) | |

After the culture reached a density of 15 OD₆₀₀, 40-50 mL/L of inoculum per initial fermentation media volume was added to the fermenter to reach a target initial OD₆₀₀ of 0.75. The fermentation media included carbohydrates, a nitrogen source, salts, and trace minerals and vitamins. The fermentation was controlled at 32° C., a pH of 6.0 with 10N sodium hydroxide, and a dissolved oxygen (DO) setpoint of 25% saturation (based on oxygen solubility at atmoshpheric pressure). At the end of batch phase, approximately 25 hours EFT, reaching an OD₆₀₀ of greater than 60 and a wet cell weight of greater than 160 g/L, a 60% w/v glycerol feed was started at a constant rate of 10 gL⁻¹ h⁻¹ (grams of glycerol per initial fermenter volume), or 16.67 mL⁻¹ h⁻¹ (volume of feed per initial fermenter volume). The fed-batch fermentation continued until approximately EFT 72 hours.

The cell culture was centrifuged and approximately two thirds of the spent fermentation medium was removed. The cells were lysed, clarified, and impurities were removed through cake filtration and ultra filtration to produce a permeate concentrate. The quantity and identity of the proteins in the permeate concentrate was determined with mass spectrometry and spectral counting.

The permeate concentrate comprised about 8.5% by weight of recombinant collagen, about 9.5% by weight of stress related proteins, about 6% by weight of cell wall related proteins, about 6.5% by weight of DNA or protein synthesis related proteins, about 20% by weight of metabolic enzyme, and about 0.01% by weight of cell integrity proteins.

The stress related proteins in the permeate concentrate included heat shock proteins (Hsp), thioredoxin, ATPases, and protein disulfide isomerases.

The cell wall related proteins in the permeate concentrate included exo-1,3-beta-glucanase, endo-beta-1,3-glucanase, glycosidase, 1,3-beta-glucanosyltransferase, chitin deacetylase, and fructose 1,6-bisphosphate aldolase.

The DNA or protein synthesis related proteins in the permeate concentrate included elongation factor 1-alpha, adenosine kinase, 60S acidic ribosomal protein P2-A, nucleoside diphosphate kinase, ribosomal protein 51, ribosomal protein L30, ribosomal protein 59, eukaryotic translation initiation factor 5A, translation initiation factor elF4A, DNA topoisomerase 2, histone methyl transferase, S-adenosyl-L-methionine-dependent tRNA, eukaryotic translation initiation factor 3 subunit 1, lysine-tRNA ligase, DNA-directed RNA polymerase subunit beta, transcription factor II, and DNA ligase.

The metabolic enzymes in the permeate included enolase I, triosephosphate isomerase, superoxide dismutase, glyceraldehyde-3-phosphate dehydrogenase, cobalamin-independent methionine synthase, 6-phosphogluconate dehydrogenase, phosphotransferase, NADPH-dependent alpha-keto amide reductase, phosphoglycerate mutase, cytoplasmic inorganic pyrophosphatase, alcohol dehydrogenase, carboxypeptidase Y inhibitor, phosphatidylglycerol/phosphatidylinositol transfer protein, cytoplasmic ATPase, mitochondrial matrix ATPase, peptidyl-prolyl cis-trans isomerase, adenosine kinase, thiol-specific peroxiredoxin, ornithine aminotransferase, ketol-acid reductoisomerse, aconitate hydratase, malate dehyrdrogenase, glycerol kinase, alanine:glyoxylate aminotransferase, nucleoside diphosphate kinase, chitin deacetylase, transketolase, NADPH oxidoreductase, fructose 1,6-bisphosphate aldolase, NADH-cytochrome b5 reductase, phosphomannomutase, mitochondrial peroxiredoxin, 3-isopropylmalate dehydrogenase, thioredoxin peroxidase, squalene epoxidase, malate dehydrogenase, coproporphyrinogen III oxidase, isocitrate dehydrogenase [NAD] subunit, catalytic subunit of NatB N-terminal acetyltransferase, carboxypeptidase, triacylglycerol lipase, adenosylhomocysteinase, flavin-containing monooxygenase, acid trehalase, high affinity methionine permease, subunit of alpha-1,6 mannosyltransferase complex, adenylate cyclase, phosphotransferase, methylenetetrahydrofolate reductase, alternative oxidase, Cu(+2)-transporting P-type ATPase, GTPase, 2-(3-amino-3carboxypropyl) histidine synthase subunit 1, peroxisome biosynthesis protein PAS1, folylpolyglutamate synthase, pyruvate kinase, serine/threonine-protein phosphatase, protein serine/threonine kinase, beta-mannosyltransferase 2, urea amidolyase, citrate synthase, and lanosterol synthase.

### Example 2: Production of Skincare Compostions

Permeate concentrate produced from Example 1 was used in the following skincare compositions.

Mask Cream

A mask cream was produced by mixing the ingredients listed in Table 3 as described herein. The percent by weight of each ingredient is listed in Table 3, wherein QS means quantity sufficient to reach 100% by weight.

TABLE 3
Mask Cream Ingredients

| | | |
|---|---|---|
| Trade name | INCI name | % |

Phase A

| | | |
|---|---|---|
| Permeate Concentrate | N/A | QS |
| Natrlquest E-30 | Trisodium Ethylenediamine Disuccinate | 0.5 |
| Zemea | Propendiol | 2.0 |
| Solagum AH | Xanthan gum, Acacia gum | 0.5 |

Phase B

| | | |
|---|---|---|
| Emulium Kappa MB | Candelilla/jojoba/rice Bran polyglyceryl- | 2.0 |
| | 3 esters, glyceryl stearate, cetearyl | |
| | alcohol, sodium stearoyl lactylate | |
| Myritol 331 | Cocoglycerides | 3.0 |
| Cetyl Alcohol | Cetyl Alcohol | 2.0 |
| Parfol 14-97 | Tetradecane | 3.0 |

Phase C

| | | |
|---|---|---|
| Baycusan C-1003 | Polyurethane-32 | 1.0 |

Phase D

| | | |
|---|---|---|
| Sensiva SC-10 | Caprylyl Glycol, Ethylhexylglycerin | 1.0 |
| Vitamin E | Tocopheryl acetate | 0.5 |
| Hydresia SF2* | Carthamus Tinctorius (Safflower) | 2.0 |
| | Oleosomes, Water | |

Solagum AH and Zemea were premixed into a slurry. Permeate concentrate was added and the solution mixed for 30 minutes until thickening occurred. Natriquest E-30 was added and Phase A was heated to 75° C.

Phase B ingredients were mixed and then heated to 75° C. to melt the ingredients. Phase B was slowly added to Phase A while mixing. The solution was homogenized for 10 minutes. Baycusan C-1003 was added and the solution homogenized for another three minutes. The solution and cooled to 45° C. while mixing. Phase D was added to the solution and mixing continued until the solution reached room temperature.

Twenty-five grams of the solution was added to a bamboo mask and sealed in a pouch.

Face Wash

A face wash was produced by mixing the ingredients listed in Table 4 as described herein. The percent by weight of each ingredient is listed in Table 4.

TABLE 4
Face Wash Ingredients

| Trade name | INCI name | %, range | %, actual |
|---|---|---|---|
| Permeate | N/A | | QS |
| Concentrate | | | |
| Zemea | Propendiol | 0.5-3 | 1.5 |
| Solagum AH | Xanthan gum, Acacia gum | 0.2-1 | 0.7 |
| SugaNate 160NC | Sodium Laurylglucosides | 15-30 | 20.0 |
| | Hydroxypropylsulfonate | | |
| Cola Teric CBS-HP | Cocamidopropyl | 7-12 | 9.0 |
| | Hydroxysultaine | | |
| Plantapon ® ACG | Sodium Cocoyl Glutamate | 15-30 | 20.0 |
| H | | | |
| Euxyl K-712 | Water, Na benzoate, K | 0.5-1.5 | 1.0 |
| | Sorbate | | |
| Phytic acid Extreme | Phytic acid | 2-5 | 4.0 |

Solagum AH and Zemea were premixed into a slurry. Permeate concentrate was added and the solution mixed for 30 minutes until thickening occurred. The remaining ingredients in Table 4 were added in order mixing well between each addition.

Eye Gel

An eye gel was produced by mixing the ingredients listed in Table 5 as described herein. The percent by weight of each ingredient is listed in Table 5, wherein QS means quantity sufficient to reach 100% by weight.

TABLE 5
Eye Gel Ingredients

| Trade name | INCI name | %, range | %, actual |
|---|---|---|---|
| Permeate Concentrate | N/A | | QS |
| Zemea | Popendiol | 1-3 | 1.5 |
| Solagum AX | Acacia Senegal gum, | 0.2-1 | 0.7 |
| | Xanthan gum | | |
| Natraquest E-30 | Trisodium | 0.2-1 | 0.5 |
| | Ethylenediamine | | |
| | Disuccinate | | |
| Euxyl 9010 | Phenoxyethanol. | 0.5-1.5 | 1.0 |
| | Ethylhexylglycerin | | |

Solagum AH and Zemea were premixed into a slurry. Permeate concentrate was added and the solution mixed for 30 minutes until thickening occurred. The remaining ingredients in Table 5 were added in order mixing well between each addition.

Day Cream

A day cream was produced by mixing the ingredients listed in Table 6 as described herein. The percent by weight of each ingredient is listed in Table 6.

TABLE 6
Day Cream Ingredients

| | | | |
|---|---|---|---|
| Trade name | INCI name | %, range | %, actual |

Phase A

| | | | |
|---|---|---|---|
| Permeate concentrate | N/A | | QS |
| Dermofeel PA-3 | Sodium Phytate | 0.5-2 | 1.0 |
| Zemea | Propendiol | 1-3 | 1.5 |
| Solagum AH | Xanthan gum, Acacia gum | 0.2-1 | 0.7 |

Phase B

| | | | |
|---|---|---|---|
| Emulium Kappa MB | Candelilla/jojoba/rice Bran polyglyceryl- | 2-6 | 4.0 |
| | 3 esters, glyceryl stearate, cetearyl | | |
| | alcohol, sodium stearoyl lactylate | | |
| Myritol 331 | Cocoglycerides | 10-20 | 15.0 |
| Cetyl Alcohol | Cetyl Alcohol | 1-4 | 2.0 |
| Parfol 14-97 | Tetradecane | 1-5 | 3.0 |

Phase C

| | | | |
|---|---|---|---|
| Baycusan C-1003 | Polyurethane-32 | 0.5-5 | 1.0 |

Phase D

| | | | |
|---|---|---|---|
| Sensiva SC10 | Caprylyl Glycol, Ethylhexylglycerin | 0.-2 | 1.0 |
| Vitamin E | Tocopheryl acetate | 0.-1 | 0.5 |
| Hydresia SF2* | Carthamus Tinctorius (Safflower) | 1-5 | 3.0 |
| | Oleosomes, Water | | |

Zemea was premixed into a slurry. The permeate concentrate was added and mixed for 30 minutes. Solagum AH and Zemea were premixed into a slurry and then added to the slurry/permeate concentrate. The solution was mixed for 30 minutes. Dermofeel PA-3 was added and Phase A was heated to 75° C.

Phase B ingredients were mixed and then heated to 75° C. to melt the ingredients. Phase B was slowly added to Phase A while mixing. The solution was homogenized for 10 minutes. Baycusan C-1003 was added and the solution homogenized for another three minutes. The solution and cooled to 45° C. while mixing. Phase D was added to the solution and mixing continued until the solution reached room temperature.

Coffee Scrub

A coffee scrub was produced by mixing the ingredients listed in Table 7 as described herein. The percent by weight of each ingredient is listed in Table 7, wherein QS means quantity sufficient to reach 100% by weight.

TABLE 7
Coffee Scrub Ingredients

| | | | |
|---|---|---|---|
| Trade name | INCI name | %, range | %, actual |

| | | |
|---|---|---|
| Base | QS | 60.0 |
| Coffee grinds | 20-60 | 40.0 |

The coffee scrub base was produced by mixing the ingredients listed in Table 8 as described herein. The percent by weight of each ingredient is listed in Table 8.

TABLE 8
Coffee Scrub Base Ingredients

| Trade name | INCI name | %, range | %, actual |
|---|---|---|---|
| Permeate Concentrate | N/A | | QS |
| Zemea | Popendiol | 0.5-3.0 | 1.5 |
| Solagum AX | Acacia Senegal gum, | 0.2-1.0 | 0.7 |
| | Xanthan gum | | |
| Dermafeel PA-3 | Sodium Phytate | 0.5-2.0 | 1.0 |
| Euxyl 9010 | Phenoxyethanol. | 0.5-1.5 | 1.0 |
| | Ethylhexylglycerin | | |

Solagum AH and Zemea were premixed into a slurry. Permeate concentrate was added and the solution mixed for 30 minutes until thickening occurred. The remaining ingredients in Table 4 were added in order mixing well between each addition.

### Example 3: Recovery of Intracellular Collagen from Cultured Pichia pastoris

Pichia pastoris cells were modified to express type III recombinant collagen, as described in Example 1. During production, Pichia pastoris cells secrete the type III recombinant collagen directly into the fermentation medium. However, a fraction of the recombinant collagen is not readily secreted, and a portion of the collagen remains inside the cell. Accordingly, processes for recovering intracellular collagen were investigated.

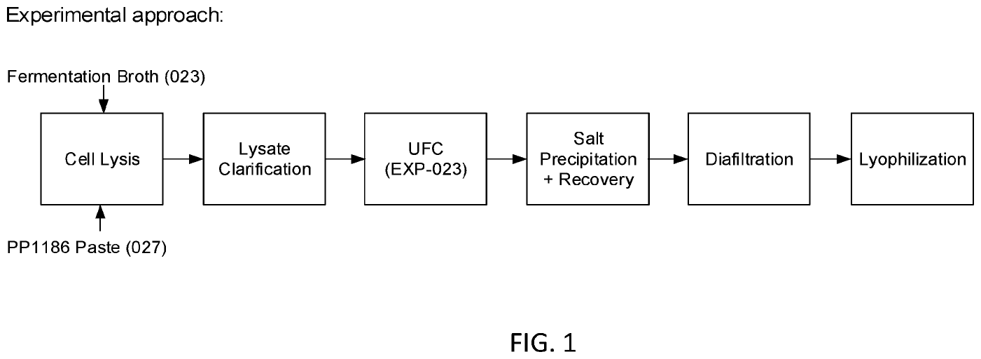

Three batches from one Pichia pastoris lot were employed in three experiments to determine the amount of recoverable intracellular collagen from a lysate of each batch. First, the amount and type of collagen was determined from a supernatant only batch and used as a comparator (Experiment 044). Second, the whole fermentation broth was subjected to cell lysis and subjected to the remaining experimental approach steps shown in the flow chart in FIG. 1 (Experiment 023). Third, a cell paste was subjected to cell lysis and to the remaining experimental approach steps shown in the flow chart in FIG. 1 (Experiment 027).

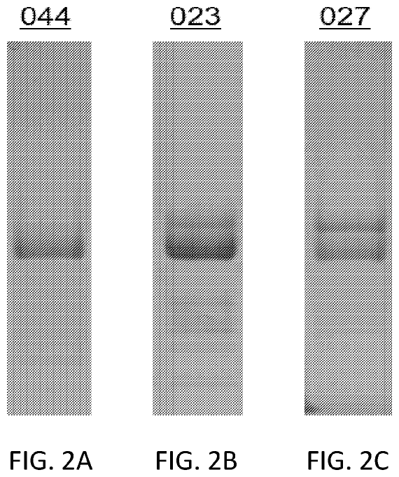

As shown in FIG. 1, a 0.5 M sodium phosphate dibasic lysis buffer solution was prepared and adjusted to pH 9+0.05 with 10 N sodium hydroxide. Then, 460.2 g of the lysis buffer was added to 3.47 kg of fermentation broth. The mixture was readjusted to pH 9 with 49.8 g of 10 N sodium hydroxide and the mixture was diluted with 520.2 g of DI water. The mixture of lysis buffer, fermentation broth and water was fed to a WAB Dyno-Mill KDL 0.6 bead (WAB US Corp., Allendale, NJ) mill to rupture the yeast cells. The bead mill settings were: 3,200 rpm, with 64 mm agitator disc which result in a 10.5 m/s tip speed. The beads were 0.6-0.8 mm zirconia silica composte beads. The bead mill was filled with 0.48 L (80% mill capacity) of beads. The mixture was fed to the bead mill at a 240 ml/min feed rate. The resulting 4.87 kg of lysate was pH adjusted to pH 4.0+0.1 with 89.2 g of 50% sulfuric acid. The lysate was clarified by centrifuge using a SORVALL centrifuge (Thermo Fisher Scientific, Waltham, MA) at 17,000 g for 20 minutes and at temperature of 10° C. This resulted in 3.08 kg of Sorvall centrifuged centrate, which was then concentrated using ultrafiltration. A Spectrum Labs Krossflow Research peristaltic TFF system (Repligen Corp., Waltham, MA) was used with a hollow fiber mPES membrane type N06-E010-10-N filter, specifically, a 10 kDa MWCO membrane filter. The filtration conditions were 10 psi TMP with a 2880 ml/min flow rate and a velocity of 0.245 m/s. The 3.08 kg of centrifuge centrate was concentrated to 1.5 kg of concentrated permeate using the TFF system. The collagen fragments (the 55 kDa and 50 kDa fragments, as shown in Experiment 023 of FIG. 2B) were then precipitated from the solution with 1.6 M sodium sulfate salt solution. 1660 g of 1.6 M sodium sulfate solution was added to 1.41 kg of concentrated permeate and mixed for at least 20 minutes. Precipitated protein was then recovered using a Sorvall centrifuge at 17,000 g for 30 minutes at 20° C., and 80.2 g of pellet wsa recovered. The pellet was resupended in 706.8 g of DI water. The diafiltration was performed using the same TTF system, conditions, and hollow fiber filter as above to remove small impurities such as salts from the product. Diafiltration was with 4.5 diavolumes of DI water. In the last step, 928 g of the diafiltration product was lyophilized.

The recovered collagen from each experiment was measured. The results are shown below in Table 9.

TABLE 9
Recovery of Intracellular Collagen

| | | Salt Precipitate | |
|---|---|---|---|
| | | Diafiltration - | Product Following |
| | Starting | Product | Lyophilization |
| Experiment | Material | rCol (g) | mass (g) |
| Experiment 023 | Broth | 16.68 | 21 |
| Experiment 027 | Paste | 6.08 | 6.1 |

Comparison of Products

Purity

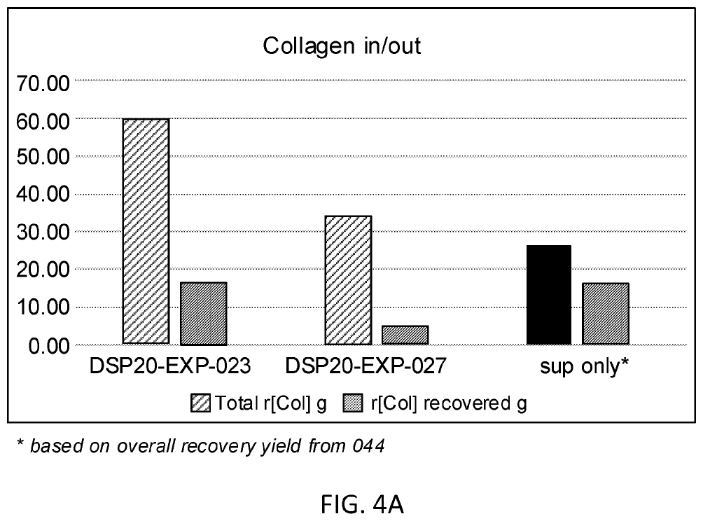

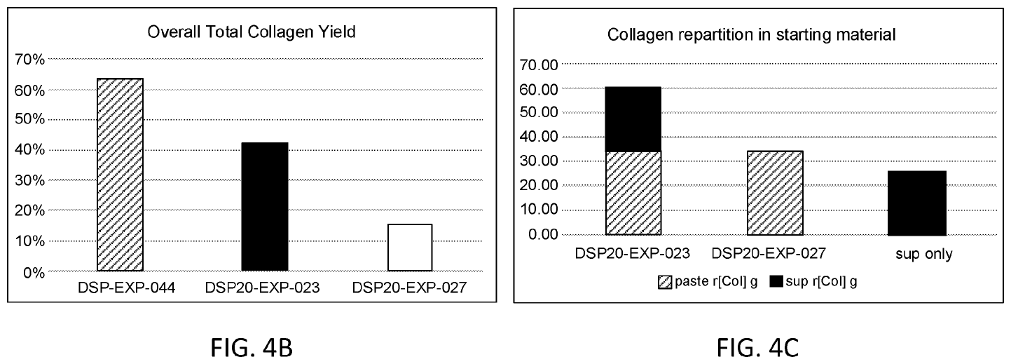

The product purity of recovered collagen from Experiments 023 and 027 was comparable to that of Experiment 044 (as shown in FIG. 4B).

Protein Fragments

Resulting protein fragments were separated by gel electrophoresis and analyzed. The analysis was performed on a 3-8% tris-acetate gel at constant voltage for 1 hour and 20 minutes. The resulting gel was stained with a colloidal commassie dye and destained with water and methanol. Upon destaining completion, the gel was analyzed on a BioRad densitometer. There was a distinct difference between the final bands, shown in FIGS. 2A-2C. The secreted supernatant product (Experiment 044, FIG. 2A) shows one collagen band, which correlates to a 50 kDa collagen fragment (SEQ ID NO: 2). In contrast, the products from Experiments 023 and 027, which include intracellular collagen, have 2 bands. One band correlates to a 55 kDa collagen fragment (SEQ ID NO: 1), which is the intracellular collagen, including the signal sequence (SEQ ID NO: 3). (FIGS. 2B and 2C.) The second band correlates to an extracellular 50 kDa fragment (SEQ ID NO: 2), which is the secreted recombinant collagen without the signal sequence. (FIGS. 2B and 2C.) It was hypothesized that the results from Experiment 27 would show the intracellular 55 kDa band only. However, the cell pellet from Experiment 027 was not thoroughly washed before processing and it is possible that pellet still contained a fraction of supernatant, which includes the 50 kDa fragment. The amino acid sequences for each collagen fragment are provided below.

55kDa Recombinant Collagen Fragment
(SEQ ID NO. 1)
MYRNLIIATALTCGAYSAYVPSEPWSTLTPDASLESALKDYSQTFGIAIK
SLDADKIKRDSYDVKSGVAVGGLAGYPGPAGPPGPPGPPGTSGHPGSPGS
PGYQGPPGEPGQAGPSGPPGPPGAIGPSGPAGKDGESGRPGRPGERGLPG
PPGIKGPAGIPGFPGMKGHRGFDGRNGEKGETGAPGLKGENGLPGENGAP
GPMGPRGAPGERGRPGLPGAAGARGNDGARGSDGQPGPPGPPGTAGFPGS
PGAKGEVGPAGSPGSNGAPGQRGEPGPQGHAGAQGPPGPPGINGSPGGKG
EMGPAGIPGAPGLMGARGPPGPAGANGAPGLRGGAGEPGKNGAKGEPGPR
GERGEAGIPGVPGAKGEDGKDGSPGEPGANGLPGAAGERGAPGFRGPAGP
NGIPGEKGPAGERGAPGPAGPRGAAGEPGRDGVPGGPGMRGMPGSPGGPG
SDGKPGPPGSQGESGRPGPPGPSGPRGQPGVMGFPGPKGNDGAPGKNGER
GGPGGPGPQGPPGKNGETGPQGPPGPTGPGGDKGDTGPPGPQGLQGLPGT
GGPPGENGKPGEPGPKGDAGAPGAPGGKGDAGAPGERGPPAIAGIGGEKA
GGFAPYYG
50kDa Recombinant Collagen Fragment
(SEQ ID NO. 2)
DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG
Signal Sequence
(SEQ ID NO: 3)
MYRNLIIATALTCGAYSAYVPSEPWSTLTPDASLESALKDYSQTFGIAIK
SLDADKIKRDSY

Results

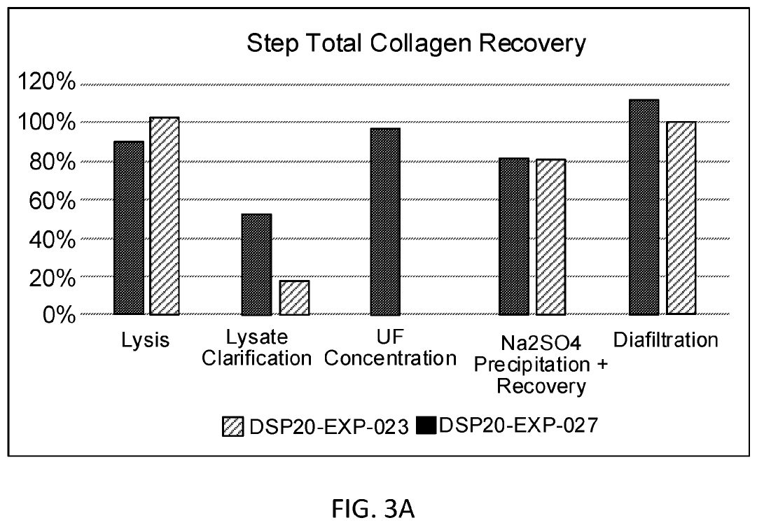

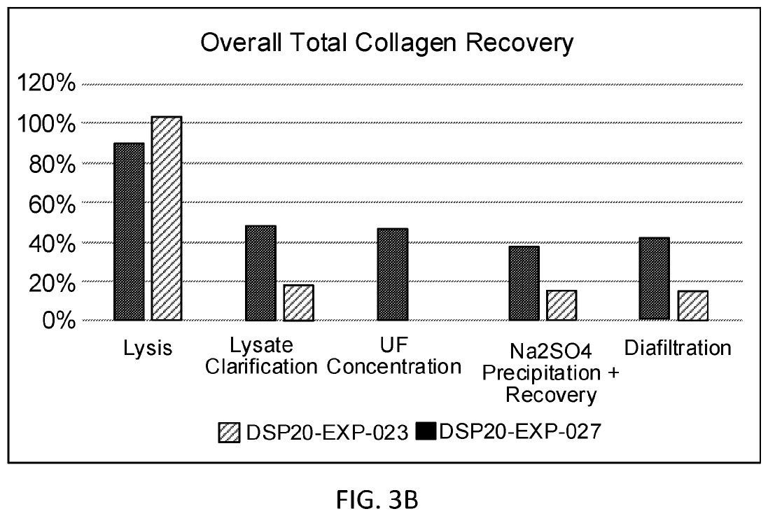

The results showed that using the fermentation broth as a starting material (Experiment 023) allowed for the recovery of more collagen coming from the supernatant, as compared to Experiment 027. The amount of collagen recovered at each step of the experimental protocol in Experiments 023 and 027 were comparable, except for the yields following the lysate clarification step, which were higher for Experiment 023. (FIGS. 3A and 3B.) The highest total recovery yield was from Experiment 044 (supernatant only). Secreted collagen was recovered less efficiently from Experiment 023 from Experiment 044, and least efficiently from Experiment 027. (FIGS. 4A-4C.)

### Example 4: Production of Skincare Compostions

Recovered instracellular collagen produced from Example 3 is used in the following skincare compositions.

Mask Cream

A mask cream is produced by mixing the ingredients listed in Table 10 as described herein. The percent by weight of each ingredient is listed in Table 10, wherein QS means quantity sufficient to reach 100% by weight.

TABLE 10
Mask Cream Ingredients

| | | |
|---|---|---|
| Trade name | INCI name | % |

Phase A

| | | |
|---|---|---|
| Recombinant collagen | N/A | QS |
| fragments disclosed | | |
| herein | | |
| Natrlquest E-30 | Trisodium Ethylenediamine Disuccinate | 0.5 |
| Zemea | Propendiol | 2.0 |
| Solagum AH | Xanthan gum, Acacia gum | 0.5 |

Phase B

| | | |
|---|---|---|
| Emulium Kappa MB | Candelilla/jojoba/rice Bran polyglyceryl- | 2.0 |
| | 3 esters, glyceryl stearate, cetearyl | |
| | alcohol, sodium stearoyl lactylate | |
| Myritol 331 | Cocoglycerides | 3.0 |
| Cetyl Alcohol | Cetyl Alcohol | 2.0 |
| Parfol 14-97 | Tetradecane | 3.0 |

Phase C

| | | |
|---|---|---|
| Baycusan C-1003 | Polyurethane-32 | 1.0 |

Phase D

| | | |
|---|---|---|
| Sensiva SC-10 | Caprylyl Glycol, Ethylhexylglycerin | 1.0 |
| Vitamin E | Tocopheryl acetate | 0.5 |
| Hydresia SF2* | Carthamus Tinctorius (Safflower) | 2.0 |
| | Oleosomes, Water | |

Face Wash

A face wash is produced by mixing the ingredients listed in Table 11 as described herein. The percent by weight of each ingredient is listed in Table 11.

TABLE 11
Face Wash Ingredients

| Trade name | INCI name | %, range | %, actual |
|---|---|---|---|
| Recombinant | N/A | | QS |
| collagen fragments | | | |
| disclosed herein | | | |
| Zemea | Propendiol | 0.5-3 | 1.5 |
| Solagum AH | Xanthan gum, Acacia gum | 0.2-1 | 0.7 |
| SugaNate 160NC | Sodium Laurylglucosides | 15-30 | 20.0 |
| | Hydroxypropylsulfonate | | |
| Cola Teric CBS-HP | Cocamidopropyl | 7-12 | 9.0 |
| | Hydroxysultaine | | |
| Plantapon ® ACG | Sodium Cocoyl Glutamate | 15-30 | 20.0 |
| H | | | |
| Euxyl K-712 | Water, Na benzoate, K | 0.5-1.5 | 1.0 |
| | Sorbate | | |
| Phytic acid Extreme | Phytic acid | 2-5 | 4.0 |

Eye Gel

An eye gel is produced by mixing the ingredients listed in Table 12 as described herein. The percent by weight of each ingredient is listed in Table 12, wherein QS means quantity sufficient to reach 100% by weight.

TABLE 12
Eye Gel Ingredients

| Trade name | INCI name | %, range | %, actual |
|---|---|---|---|
| Recombinant collagen | N/A | | QS |
| fragments disclosed | | | |
| herein | | | |
| Zemea | Popendiol | 1-3 | 1.5 |
| Solagum AX | Acacia Senegal gum, | 0.2-1 | 0.7 |
| | Xanthan gum | | |
| Natraquest E-30 | Trisodium | 0.2-1 | 0.5 |
| | Ethylenediamine | | |
| | Disuccinate | | |
| Euxyl 9010 | Phenoxyethanol. | 0.5-1.5 | 1.0 |
| | Ethylhexylglycerin | | |

Day Cream

A day cream is produced by mixing the ingredients listed in Table 13 as described herein. The percent by weight of each ingredient is listed in Table 13.

TABLE 13
Day Cream Ingredients

| | | | |
|---|---|---|---|
| Trade name | INCI name | %, range | %, actual |

Phase A

| | | | |
|---|---|---|---|
| Recombinant collagen | N/A | | QS |
| fragments disclosed | | | |
| herein | | | |
| Dermofeel PA-3 | Sodium Phytate | 0.5-2 | 1.0 |
| Zemea | Propendiol | 1-3 | 1.5 |
| Solagum AH | Xanthan gum, Acacia gum | 0.2-1 | 0.7 |

Phase B

| | | | |
|---|---|---|---|
| Emulium Kappa MB | Candelilla/jojoba/rice Bran polyglyceryl- | 2-6 | 4.0 |
| | 3 esters, glyceryl stearate, cetearyl | | |
| | alcohol, sodium stearoyl lactylate | | |
| Myritol 331 | Cocoglycerides | 10-20 | 15.0 |
| Cetyl Alcohol | Cetyl Alcohol | 1-4 | 2.0 |
| Parfol 14-97 | Tetradecane | 1-5 | 3.0 |

Phase C

| | | | |
|---|---|---|---|
| Baycusan C-1003 | Polyurethane-32 | 0.5-5 | 1.0 |

Phase D

| | | | |
|---|---|---|---|
| Sensiva SC10 | Caprylyl Glycol, Ethylhexylglycerin | 0.-2 | 1.0 |
| Vitamin E | Tocopheryl acetate | 0.-1 | 0.5 |
| Hydresia SF2* | Carthamus Tinctorius (Safflower) | 1-5 | 3.0 |
| | Oleosomes, Water | | |

Coffee Scrub

A coffee scrub is produced by mixing the ingredients listed in Table 14 as described herein. The percent by weight of each ingredient is listed in Table 14, wherein QS means quantity sufficient to reach 100% by weight.

TABLE 14
Coffee Scrub Ingredients

| | | | |
|---|---|---|---|
| Trade name | INCI name | %, range | %, actual |

| | | |
|---|---|---|
| Base | QS | 60.0 |
| Coffee grinds | 20-60 | 40.0 |

The coffee scrub base is produced by mixing the ingredients listed in Table 15 as described herein. The percent by weight of each ingredient is listed in Table 15.

TABLE 15
Coffee Scrub Base Ingredients

| Trade name | INCI name | %, range | %, actual |
|---|---|---|---|
| Recombinant collagen | N/A | | QS |
| fragments disclosed | | | |
| herein | | | |
| Zemea | Popendiol | 0.5-3.0 | 1.5 |
| Solagum AX | Acacia Senegal gum, | 0.2-1.0 | 0.7 |
| | Xanthan gum | | |
| Dermafeel PA-3 | Sodium Phytate | 0.5-2.0 | 1.0 |
| Euxyl 9010 | Phenoxyethanol. | 0.5-1.5 | 1.0 |
| | Ethylhexylglycerin | | |

Alcohol-Based Toner

An alcohol-based toner and a water-based toner are produced by mixing the ingredients listed in Tables 16 and 17.

TABLE 16
Alcohol-based toner

| INGREDIENTS: | % WT/WT |
|---|---|
| Water (Aqua) | Remainder Ingredient |
| Recombinant collagen fragments | about 0.0005% to about 25% |
| disclosed herein | |
| Alcohol Denat. | 10-20% |
| Pentylene Glycol | 5-10% |
| Glycerin | 1-5% |
| Gluconolactone | 0.1-1% |
| Dipotassium Glycyrrhizate | 0.1-1% |
| Sodium Citrate | 0.1-1% |
| Sodium Benzoate | 0.1-1% |

TABLE 17
Water-based toner

| INGREDIENTS: | % WT/WT |
|---|---|
| Water (Aqua) | Remainder Ingredient |
| Recombinant collagen fragments | about 0.0005% to about 25% |
| disclosed herein | |
| Niacinamide | 1-5% |
| Pentylene Glycol | 1-5% |
| Propanediol | 1-5% |
| Glycerin | 1-5% |
| Biosaccharide Gum-1 | 0.1-1% |
| Glyceryl Caprylate | 0.1-1% |
| Sodium Anisate | 0.1-1% |
| Sodium Hydroxide | 0.1-1% |
| Caprylhydroxamic Acid | 0.1-1% |
| Acrylates/C10-30 Alkyl Acrylate | 0.1-1% |
| Crosspolymer | |
| Sodium Levulinate | 0.1-1% |
| Caprylyl Glycol | 0.1-1% |

The compositions described herein are formulated into a cream, a gel, or a serum. Exemplary cream, gel, and serum formulations are set forth below.

TABLE 18
Cream

| INGREDIENTS: | % WT/WT |
|---|---|
| Water (Aqua) | Remainder Ingredient |
| Recombinant collagen fragments | about 0.0005% to about 25% |
| disclosed herein | |
| Cetearyl Alcohol | 5-10% |
| Glycerin | 1-5% |
| Squalane | 1-5% |
| Butyrospermum Parkii (Shea) | 1-5% |
| Butter | |
| Glyceryl Caprylate | 1-5% |
| Microcrystalline Cellulose | 1-5% |
| Glyceryl Stearate Citrate | 0.1-1% |
| Tocopheryl Acetate | 0.1-1% |
| Cetearyl Glucoside | 0.1-1% |
| Sodium Stearoyl Glutamate | 0.1-1% |
| Cellulose Gum | 0.1-1% |
| Xanthan Gum | 0.1-1% |
| Caprylhydroxamic Acid | 0.1-1% |
| Sodium Phytate | 0.01-0.1% |

TABLE 19
Gel

| INGREDIENTS: | % WT/WT |
|---|---|
| Water (Aqua) | Remainder Ingredient |
| Recombinant collagen fragments | about 0.0005% to about 25% |
| disclosed herein | |
| Sodium Phytate | 0.1-1% |
| Sodium Hydroxide | 0.1-1% |
| Carbomer | 0.1-1% |
| Phenoxyethanol | 0.1-1% |

TABLE 20
Serum

| INGREDIENTS: | % WT/WT |
|---|---|
| Water (Aqua) | Remainder Ingredient |
| Recombinant collagen fragments | about 0.0005% to about 25% |
| disclosed herein | |
| Pentylene Glycol | 1-5% |
| Niacinamide | 1-5% |
| Dimethicone | 1-5% |
| Propanediol | 1-5% |
| Tocopherol | 0.1-1% |
| Sodium Hyaluronate | 0.1-1% |
| Linoleic Acid | 0.1-1% |
| Ammonium | 0.1-1% |
| Acryloyldimethyltaurate/VP | |
| Copolymer | |
| Acrylates/C10-30 Alkyl Acrylate | 0.1-1% |
| Crosspolymer | |
| Caprylyl Glyceryl Ether | 0.1-1% |
| Tetrasodium Glutamate Diacetate | 0.01-0.1% |
| Sodium Hydroxide | 0.01-0.1% |
| Phenoxyethanol | 0.01-0.1% |
| Linolenic Acid | 0.001-0.0 |

The compositions described herein are formulated as a shampoo and conditioner. Exemplary shampoo or conditioner formulations are set forth below.

TABLE 21
Shampoo

| INGREDIENTS: | % WT/WT |
|---|---|
| Water (Aqua) | Remainder Ingredient |
| Recombinant collagen fragments | about 0.0005% to about 25% |
| disclosed herein | |
| Cocamidopropyl Betaine | 5-10% |
| Sodium Lauroyl Methyl Isethionate | 5-10% |
| Propanediol | 1-5% |
| Sodium Methyl Oleoyl Taurate | 1-5% |
| Sodium Cocoyl Isethionate | 1-5% |
| Trisodium Ethylenediamine | 0.1-1% |
| Disuccinate | |
| Caprylhydroxamic Acid | 0.1-1% |
| Panthenol | 0.1-1% |
| Citric Acid | 0.1-1% |
| Caprylyl Glycol | 0.1-1% |
| Sodium Benzoate | 0.1-1% |

TABLE 22
Conditioner

| INGREDIENTS: | % WT/WT |
|---|---|
| Water (Aqua) | Remainder Ingredient |
| Recombinant collagen | about 0.0005% to about 25% |
| fragments disclosed herein | |
| Cetearyl Alcohol | 5-10% |
| Glyceryl Caprylate | 1-5% |
| Behentrimonium Methosulfate | 1-5% |
| Glycerin | 1-5% |
| Caprylhydroxamic Acid | 0.1-1% |
| Panthenol | 0.1-1% |
| Hydroxyethylcellulose | 0.1-1% |
| Cocos Nucifera (Coconut) Oil | 0.1-1% |
| Citric Acid | 0.01-0.1% |

### Example 5: Stimulation of Collagen I and Collagen III Synthesis Recombinant Collagen Fragments in Fibroblast Cells

A fibroblast cell culture model is used to assess the ability of the test materials to exert an effect on collagen synthesis. This study also assesses the viability of the cells after exposure to the test materials.

MTT Assay

Changes in cell number can be assessed via an MTT assay. The MTT assay is a colorimetric analysis of the metabolic activity of the cell, which is a reflection of the number of viable cells. Reduction of MTT by mitochondria results in the formation of insoluble purple formazin crystals that are extracted from the cells with isopropanol and quantified spectrophotometrically. The intensity of the purple color is directly proportional to the metabolic activity of the cells and inversely proportional to the toxicity of the test material.

Fibroblasts are seeded into the individual wells of a 24-well plate in 0.5 ml of Fibroblast Growth Media (FGM) and incubated overnight at 37±2° C. and 5±1% CO₂. On the following day the media is removed via aspiration to eliminate any non-adherent cells and replaced with 0.5 ml of fresh FGM. The cells were grown until confluent, with a media change every 48 to 72 hours. Upon reaching confluency the cells are treated for 24 hours with DMEM supplemented with 1.5% FBS to wash out any effects from the growth factors included in the normal culture media. After this 24-hour wash out period the cells are treated with the test materials at the specified concentrations, dissolved in FGM with 1.5% FBS. TGF-B (50 ng/ml) is used as a positive control for inducing collagen expression. Untreated cells (negative controls) recieve DMEM with 1.5% FBS. As a negative control, cells are treated with 100 μM bDcAMP in FGM with 1.5% FBS. The cells are incubated for 48 hours and at the end of the incubation period cell culture medium is collected and either stored frozen (−75° C.) or assayed immediately. Materials are tested in triplicate.

The samples tested are the 55 kDa recombinant collagen fragment described herein (SEQ ID NO: 1), full-length bovine collagen 3 with hydroxylation (7%) (“full-length bcol 3”), marine collagen (Ashland), acacia collagen (Lipoid Kosmetik AG), recombinant human collagen 21 (Geltor) (“HumCo1121”), BIOLLAGEN (Jland Biotech), and full length type III bovine collagen with hydroxylation (45%) (“full-length bcol3”). Each collagen sample is diluted at a range of percent concentrations in the tissue culture media, FGM with 1.5%. The 55 kDa recombinant collagen fragment is tested at 0.1%, 0.05%, 0.01%, 0.005%, and 0.001% by weight in the culture media. The full length type III bovine collagen with 7% hydroxylation is tested at 0.05%, 0.01%, 0.005%, 0.001%, and 0.0005% by weight in the culture media. The marine collagen is tested at 1%, 0.5%, 0.01%, 0.05%, and 0.01% by weight in the culture media. The acacia collagen is tested at 1%, 0.5%, 0.01%, 0.05%, and 0.01% by weight in the culture media. The human collagen 21 is tested at 0.1%, 0.05%, 0.01%, 0.005%, and 0.001% by weight in the culture media. The human collagen 21 is tested at 0.1%, 0.05%, 0.01%, 0.005%, and 0.001% by weight in the culture media. The human collagen 21 is tested at 0.1%, 0.05%, 0.01%, 0.005%, and 0.001% by weight in the culture media. The full length bovine type III collagen with 45% hydroxylation is tested at 0.015%, 0.003%, 0.0015%, 0.0003%, and 0.00015% by weight in the culture media.

After the 2-day incubation, the cell culture medium is removed and the fibroblasts are washed twice with PBS to remove any remaining test material. After the final wash, 500 μl of DMEM supplemented with 0.5 mg/ml MTT is added to each well and the cells are incubated for 1 hour at 37±2° C. and 5±1% CO₂. After the incubation, the DMEM/MTT solution is removed and the cells are washed again once with PBS and then 0.5 ml of isopropyl alcohol is added to the well to extract the purple formazin crystals. Two hundred microliters of the isopropyl extracts are transferred to a 96-well plate and the plate is read at 540 nm using isopropyl alcohol as a blank. The mean MTT absorbance value for the negative control cells is calculated and used to represent 100% cell viability. The individual MTT values from the cells undergoing the various treatments are then divided by the mean value for the negative control cells and expressed as a percent to determine the change in cell viability caused by each treatment.

None of the tested collagens are observed to impact cell viability. Each treated sample of cells are at least as viable as untreated control cells.

Type I Collagen Assay

Fibroblasts are the main source of the extracellular matrix peptides, including collagen. Procollagen is a large peptide synthesized by fibroblasts in the dermal layer of the skin and is the precursor for collagen. As procollagen is processed to form a mature collagen protein, a propeptide portion is cleaved off in the form of a type I C-peptide. Both the mature collagen protein and the type I C-peptide fragment are then released into the extracellular environment. As collagen is synthesized the type I C-peptide fragment accumulates in the tissue culture medium. Since there is a 1:1 stoichiometric ratio between the two parts of the procollagen peptide, assaying for type I C-peptide reflects the amount of collagen synthesized. To measure the effects of different forms of collagen on collagen synthesis and secretion, Type 1 C-peptide is assayed via an ELISA based method.

Fibroblasts are seeded into the individual wells of a 24-well plate in 0.5 ml of Fibroblast Growth Media (FGM) and incubated overnight at 37±2° C. and 5±1% CO₂. On the following day the media is removed via aspiration to eliminate any non-adherent cells and replaced with 0.5 ml of fresh FGM. The cells are grown until confluent, with a media change every 48 to 72 hours. Upon reaching confluency the cells are treated for 24 hours with DMEM supplemented with 1.5% FBS to wash out any effects from the growth factors included in the normal culture media. After this 24-hour wash out period the cells are treated with the test materials at the specified concentrations, dissolved in FGM with 1.5% FBS. TGF-B (50 ng/ml) is used as a positive control for inducing collagen expression. Untreated cells (negative controls) just receive DMEM with 1.5% FBS. The cells are incubated for 48 hours and at the end of the incubation period cell culture medium is collected and either stored frozen (−75° C.) or assayed immediately. Materials are tested in triplicate.

The samples tested are the 55 kDa recombinant collagen fragment described herein, full-length bovine collagen 3 with hydroxylation (7%), marine collagen (Ashland), acacia collagen (Lipoid Kosmetik AG), recombinant human collagen 21 (Geltor), BIOLLAGEN (Jland Biotech), and full length type III bovine collagen with hydroxylation (45%). Each collagen sample is diluted at a range of percent concentrations in the tissue culture media, FGM with 1.5% FBS (see FIG. 11A and FIG. 11B). The 55 kDa recombinant collagen fragment described herein (SEQ ID NO:1) is tested at 0.1%, 0.05%, 0.01%, 0.005%, and 0.001% by weight in the culture media. The full length type III bovine collagen with 7% hydroxylation is tested at 0.05%, 0.01%, 0.005%, 0.001%, and 0.0005% by weight in the culture media. The marine collagen is tested at 1%, 0.5%, 0.01%, 0.05%, and 0.01% by weight in the culture media. The acacia collagen is tested at 1%, 0.5%, 0.01%, 0.05%, and 0.01% by weight in the culture media. The human collagen 21 is tested at 0.1%, 0.05%, 0.01%, 0.005%, and 0.001% by weight in the culture media. The human collagen 21 is tested at 0.1%, 0.05%, 0.01%, 0.005%, and 0.001% by weight in the culture media. The human collagen 21 is tested at 0.1%, 0.05%, 0.01%, 0.005%, and 0.001% by weight in the culture media. The full length bovine type III collagen with 45% hydroxylation is tested at 0.015%, 0.003%, 0.0015%, 0.0003%, and 0.00015% by weight in the culture media.

For the ELISA assay, a series of type I C-peptide standards is prepared ranging from 0 ng/ml to 640 ng/ml. Next, an ELISA microplate is prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard is then added to appropriate wells and the microplate was covered and allowed to incubate for 3±0.25 hours at 37° C.

After the incubation the wells are aspirated and washed three times with 400 μl of wash buffer. After the last wash is removed 100 μl of peroxidase substrate solution (hydrogen peroxide+tetramethylbenzidine as a chromagen) is added to each well and the plate is incubated for 15±5 minutes at room temperature. After the incubation 1001 μl of stop solution (1 N sulfuric acid) is added to each well and the plate was read using a microplate reader at 450 nm. To quantify the amount of each substance present, a standard curve is generated using known concentrations of each substance. A regression analysis is performed to establish the line that best fits these data points. Absorbance values for the test materials and untreated samples are used to estimate the amount of each substance present in each sample. Treatment means were compared using an ANOVA, with an n=3 per treatment. Statistical significance was set at p<0.05.

The 55 kDa recombinant collagen fragment described herein (SEQ ID NO: 1) increases the amount of Type I collagen secreted by the treated fibroblasts. Treatment with 0.1% by weight (final concentration) of the 55 kDa fragment of human collagen increases collagen I expression by over 200% relative to untreated cells. The cells are separately treated with equivalent or even higher concentrations of a range of different collagens. The cells are treated with a range of collagens of similar size to, or smaller than, the 55 kDa recombinant collagen fragment, particularly hydrolyzed marine collagen, acacia collagen, human collagen 21, and BIOLLAGEN. These collagens of similar size have no effect on collagen I production, even when those collagens are tested at up to 10-fold higher percent concentrations than the 55 kDa fragment. The cells are treated with full length bovine collagen III, both when it is approximately 7% hydroxylated and when it is approximately 45% hydroxylated. Full length bovine collagen III is able to induce collagen I expression, but only when it was highly hydroxylated (˜45%). Therefore, compositions comprising the 55 kDa fragment of human collagen are useful when increased production of type I collagen is desired.

Type III Collagen Assays

Type III collagen is synthesized by dermal fibroblasts as a large pro-peptide. As the peptide is processed to form a mature type III collagen protein, a pro-peptide portion is cleaved off (type III N-peptide). Both the mature collagen protein and the type III N-peptide fragment are then released into the extracellular environment. As collagen is synthesized the type III N-peptide fragment accumulates in the tissue culture medium. Since there is a 1:1 stoichiometric ratio between the two parts of the procollagen peptide, assaying for type III N-peptide reflects the amount of collagen synthesized. To measure the effects of different forms of collagen on collagen synthesis and secretion, Type III N-peptide is assayed via an ELISA based method.

Fibroblasts are seeded into the individual wells of a 24-well plate in 0.5 ml of Fibroblast Growth Media (FGM) and incubated overnight at 37±2° C. and 5±1% CO₂. On the following day the media is removed via aspiration to eliminate any non-adherent cells and replaced with 0.5 ml of fresh FGM. The cells are grown until confluent, with a media change every 48 to 72 hours. Upon reaching confluency the cells are treated for 24 hours with DMEM supplemented with 1.5% FBS to wash out any effects from the growth factors included in the normal culture media. After this 24-hour wash out period the cells are treated with the test materials described below at the specified concentrations, dissolved in FGM with 1.5% FBS. TGF-B (50 ng/ml) is used as a positive control for inducing collagen expression. Untreated cells (negative controls) just receive DMEM with 1.5% FBS. The cells are incubated for 48 hours and at the end of the incubation period cell culture medium is collected and either stored frozen (−75° C.) or assayed immediately. Materials were tested in triplicate.

The samples tested were are the 55 kDa recombinant collagen fragment described herein (SEQ ID NO: 1), full-length bovine collagen 3 with hydroxylation (7%), marine collagen (Ashland), acacia collagen (Lipoid Kosmetik AG), recombinant human collagen 21 (Geltor), BIOLLAGEN (Jland Biotech), and full length type III bovine collagen with hydroxylation (45%). Each collagen sample is diluted at a range of percent concentrations in the tissue culture media, FGM with 1.5% FBS. The the 55 kDa recombinant collagen fragment described herein (SEQ ID NO: 1) is tested at 0.1%, 0.05%, 0.01%, 0.005%, and 0.001% by weight in the culture media. The full length type III bovine collagen with 7% hydroxylation is tested at 0.05%, 0.01%, 0.005%, 0.001%, and 0.0005% volume in the culture media. The marine collagen is tested at 1%, 0.5%, 0.01%, 0.05%, and 0.01% volume in the culture media. The acacia collagen is tested at 1%, 0.5%, 0.01%, 0.05%, and 0.01% volume in the culture media. The human collagen 21 is tested at 0.1%, 0.05%, 0.01%, 0.005%, and 0.001% volume in the culture media. The human collagen 21 is tested at 0.1%, 0.05%, 0.01%, 0.005%, and 0.001% volume in the culture media. The human collagen 21 is tested at 0.1%, 0.05%, 0.01%, 0.005%, and 0.001% volume in the culture media. The full length bovine type III collagen with 45% hydroxylation is tested at 0.015%, 0.003%, 0.0015%, 0.0003%, and 0.00015% volume in the culture media.

For the ELISA assay, a series of standards is prepared and 100 μl of these standards or samples are added to the wells of the type III collagen ELISA plates. The plates are then incubated at 37° C. for 1.5 hours. After this incubation the ELISA plates are then washed twice with wash buffer, followed by the application of 100 μl of detection antibody solution. The ELISA plates are then incubated for 1 hour at 37° C. After the incubation all of the ELISA plates are washed with wash solution followed by the addition of 100 μl of HRP conjugate solution and incubated at 37° C. for 30 minutes. After this incubation the ELISA plates are again washed and 100 μl of substrate solution is added to each well and the well-plates are incubated for 10-30 minutes at room temperature to allow the color generation reaction to occur. At the end of the color generation reaction 100 μl of stop solution is added to each well and the plates were read at 460 nm using a plate reader. To quantify the amount of each substance present, a standard curve is generated using known concentrations of each substance. A regression analysis is performed to establish the line that best fits these data points. Absorbance values for the test materials and untreated samples are used to estimate the amount of each substance present in each sample. Treatment means are compared using an ANOVA, with an n=3 per treatment. Statistical significance is set at p<0.05.

The cells are treated with a range of percent by weight concentrations of each collagen solution. The results show that treatment with 0.1% the 55 kDa recombinant collagen fragment described herein unexpectedly increased the amount of Type III collagen secreted by the treated fibroblasts by over 200% relative to untreated cells. Therefore, compositions comprising the 55 kDa fragment of human collagen are useful when increased production of type III collagen is desired.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A recombinant collagen fragment having a molecular weight of about 55 kDa and a sequence identity of at least about 85% to the amino acid sequence set forth in SEQ ID NO: 1.

2. The recombinant collagen fragment of claim 1, wherein the collagen fragment has the amino acid sequence set forth in SEQ ID NO: 1.

3. A composition comprising the recombinant collagen fragment of claim 1 or 2.

4. The composition of claim 3, wherein the composition further comprises a recombinant collagen fragment having the amino acid sequence set forth in SEQ ID NO: 2.

5. The composition of claim 3 or 4, further comprising a pharmaceutically acceptable or cosmetically acceptable excipient.

6. A method of treating a dermatological condition comprising topically administering an effective amount of the recombinant collagen fragment of claim 1 or 2 to a subject in need thereof.

7. A method of treating a dermatological condition comprising topically administering an effective amount of the composition of claim 4 or 5 to a subject in need thereof.

8. The method of claim 7, wherein the dermatological condition is selected from the group consisting of fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, rhinophyma, and combinations thereof.

9. The method of any one of claims 6-8, wherein the composition is topically administered to an area of skin selected from the group consisting of a facial surface, scalp, neck, ears, shoulders, chest (including breasts and/or the décolletage), arms, hands, legs, stomach, buttocks, groin, back, feet, and combinations thereof.

10. A method of producing the recombinant collagen fragment of claim 1 or 2, comprising producing the recombinant collagen fragment in a genetically engineered strain of yeast.

11. The method of claim 10, wherein the yeast is Pichia pastoris.

12. The method of claim 10 or 11, wherein the method comprises:
(i) fermenting a genetically engineered yeast in a fermentation broth;
(ii) recovering from the supernatant of the fermentation broth recombinant collagen fragments secreted by the genetically engineered yeast; and
(iii) optionally, purifying the recombinant collagen fragments.

13. The method of claim 10 or 11, wherein the method comprises:
(i) fermenting a genetically engineered yeast in a fermentation broth;
(ii) recovering recombinant collagen fragments not secreted into the supernatant by the genetically engineered yeast; and
(iii) optionally, purifying the recombinant collagen fragments.

14. The method of claim 13, wherein the recovering the recombinant collagen fragments not secreted into the supernatant comprises recovering the intracellular recombinant collagen fragments from a cell paste.

15. The method of any one of claims 10-15, further comprising lyophilizing the recovered recombinant collagen fragments.

16. A skincare product comprising the recombinant collagen fragment of claim 1 or 2, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

17. A skincare product comprising the composition of any one of claims 3-5, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

18. A method of treating a wound in a human subject in need thereof, the method comprising applying the composition of any one of claims 3-5 to the wound on the subject, wherein applying the recombinant collagen fragment induces the production of human Type I collagen, human Type III collagen, or a combination thereof.

19. The method of claim 19, wherein the collagen fragment is topically applied to the wound.

20. An aqueous skincare composition comprising water and the recombinant collagen fragment of claim 1 or 2, at least one stress related protein, at least one cell wall related protein, at least one DNA or protein synthesis related protein, at least one metabolic enzyme, and, optionally, at least one cell integrity protein.

21. A method of treating an area of skin comprising topically applying an effective amount of the composition of claim 21, to the area of skin.

22. The method of claim 22, wherein the area of skin is selected from the group consisting of a facial surface, scalp, neck, ears, shoulders, chest (including breasts and/or the décolletage), arms, hands, legs, stomach, buttocks, groin, back, feet, and combinations thereof.

23. The method of claim 23, wherein the facial surface is selected from the group consisting of forehead, eyes, a perioral surface, a chin surface, a periorbital surface, a nasal surface, a cheek skin surface, and combinations thereof.
